SEQUENCE LISTING (22 sequences)

SEQ ID NO: 1 - protein, 1331 aa, Homo sapiens
TVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 2 - protein, 1328 aa, Homo sapiens
AGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 3 - protein, 732 aa, Homo sapiens
LQEVHVSKETIGKISAASKMMWCSAAVDIMFLLDGSNSVGKGSFERSKHFAITVCDGLDISPERVRVGAFQFSSTPHLEFPLDSFSTQQEVKARIKRMVFKGGRTETELALKYLLHRGLPGGRNASVPQILIIVTDGKSQGDVALPSKQLKERGVTVFAVGVRFPRWEELHALASEPRGQHVLLAEQVEDATNGLFSTLSSSAICSSATPDCRVEAHPCEHRTLEMVREFAGNAPCWRGSRRTLAVLAAHCPFYSWKRVFLTHPATCYRTTCPGPCDSQPCQNGGTCVPEGLDGYQCLCPLAFGGEANCALKLSLECRVDLLFLLDSSAGTTLDGFLRAKVFVKRFVRAVLSEDSRARVGVATYSRELLVAVPVGEYQDVPDLVWSLDGIPFRGGPTLTGSALRQAAERGFGSATRTGQDRPRRVVVLLTESHSEDEVAGPARHARARELLLLGVGSEAVRAELEEITGSPKHVMVYSDPQDLFNQIPELQGKLCSRQRPGCRTQALDLVFMLDTSASVGPENFAQMQSFVRSCALQFEVNPDVTQVGLVVYGSQVQTAFGLDTKPTRAAMLRAISQAPYLGGVGSAGTALLHIYDKVMTVQRGARPGVPKAVVVLTGGRGAEDAAVPAQKLRNNGISVLVVGVGPVLSEGLRRLAGPRDSLIHVAAYADLRYHQDVLIEWLCGEAKQPVNLCKPSPCMNEGSCVLQNGSYRCKCRDGWEGPHCENRFLRRP

SEQ ID NO: 4 - DNA, 4171 bp, Homo sapiens
cgtgacactgtctcggctacagacccagagggagcacactgccaggatgggagctgctgggaggcaggacttcctcttcaaggccatgctgaccatcagctggctcactctgacctgcttccctggggccacatccacagtggctgctgggtgccctgaccagagccctgagttgcaaccctggaaccctggccatgaccaagaccaccatgtgcatatcggccagggcaagacactgctgctcacctcttctgccacggtctattccatccacatctcagagggaggcaagctggtcattaaagaccacgacgagccgattgttttgcgaacccggcacatcctgattgacaacggaggagagctgcatgctgggagtgccctctgccctttccagggcaatttcaccatcattttgtatggaagggctgatgaaggtattcagccggatccttactatggtctgaagtacattggggttggtaaaggaggcgctcttgagttgcatggacagaaaaagctctcctggacatttctgaacaagacccttcacccaggtggcatggcagaaggaggctatttttttgaaaggagctggggccaccgtggagttattgttcatgtcatcgaccccaaatcaggcacagtcatccattctgaccggtttgacacctatagatccaagaaagagagtgaacgtctggtccagtatttgaacgcggtgcccgatggcaggatcctttctgttgcagtgaatgatgaaggttctcgaaatctggatgacatggccaggaaggcgatgaccaaattgggaagcaaacacttcctgcaccttggatttagacacccttggagttttctaactgtgaaaggaaatccatcatcttcagtggaagaccatattgaatatcatggacatcgaggctctgctgctgcccgggtattcaaattgttccagacagagcatggcgaatatttcaatgtttctttgtccagtgagtgggttcaagacgtggagtggacggagtggttcgatcatgataaagtatctcagactaaaggtggggagaaaatttcagacctctggaaagctcacccaggaaaaatatgcaatcgtcccattgatatacaggccactacaatggatggagttaacctcagcaccgaggttgtctacaaaaaaggccaggattataggtttgcttgctacgaccggggcagagcctgccggagctaccgtgtacggttcctctgtgggaagcctgtgaggcccaaactcacagtcaccattgacaccaatgtgaacagcaccattctgaacttggaggataatgtacagtcatggaaacctggagataccctggtcattgccagtactgattactccatgtaccaggcagaagagttccaggtgcttccctgcagatcctgcgcccccaaccaggtcaaagtggcagggaaaccaatgtacctgcacatcggggaggagatagacggcgtggacatgcgggcggaggttgggcttctgagccggaacatcatagtgatgggggagatggaggacaaatgctacccctacagaaaccacatctgcaatttctttgacttcgatacctttgggggccacatcaagtttgctctgggatttaaggcagcacacttggagggcacggagctgaagcatatgggacagcagctggtgggtcagtacccgattcacttccacctggccggtgatgtagacgaaaggggaggttatgacccacccacatacatcagggacctctccatccatcatacattctctcgctgcgtcacagtccatggctccaatggcttgttgatcaaggacgttgtgggctataactctttgggccactgcttcttcacggaagatgggccggaggaacgcaacacttttgaccactgtcttggcctccttgtcaagtctggaaccctcctcccctcggaccgtgacagcaagatgtgcaagatgatcacagaggactcctacccagggtacatccccaagcccaggcaagactgcaatgctgtgtccaccttctggatggccaatcccaacaacaacctcatcaactgtgccgctgcaggatctgaggaaactggattttggtttatttttcaccacgtaccaacgggcccctccgtgggaatgtactccccaggttattcagagcacattccactgggaaaattctataacaaccgagcacattccaactaccgggctggcatgatcatagacaacggagtcaaaaccaccgaggcctctgccaaggacaagcggccgttcctctcaatcatctctgccagatacagccctcaccaggacgccgacccgctgaagccccgggagccggccatcatcagacacttcattgcctacaagaaccaggaccacggggcctggctgcgcggcggggatgtgtggctggacagctgccggtttgctgacaatggcattggcctgaccctggccagtggtggaaccttcccgtatgacgacggctccaagcaagagataaagaacagcttgtttgttggcgagagtggcaacgtggggacggaaatgatggacaataggatctggggccctggcggcttggaccatagcggaaggaccctccctataggccagaattttccaattagaggaattcagttatatgatggccccatcaacatccaaaactgcactttccgaaagtttgtggccctggagggccggcacaccagcgccctggccttccgcctgaataatgcctggcagagctgcccccataacaacgtgaccggcattgcctttgaggacgttccgattacttccagagtgttcttcggagagcctgggccctggttcaaccagctggacatggatggggataagacatctgtgttccatgacgtcgacggctccgtgtccgagtaccctggctcctacctcacgaagaatgacaactggctggtccggcacccagactgcatcaatgttcccgactggagaggggccatttgcagtgggtgctatgcacagatgtacattcaagcctacaagaccagtaacctgcgaatgaagatcatcaagaatgacttccccagccaccctctttacctggagggggcgctcaccaggagcacccattaccagcaataccaaccggttgtcaccctgcagaagggctacaccatccactgggaccagacggcccccgccgaactcgccatctggctcatcaacttcaacaagggcgactggatccgagtggggctctgctacccgcgaggcaccacattctccatcctctcggatgttcacaatcgcctgctgaagcaaacgtccaagacgggcgtcttcgtgaggaccttgcagatggacaaagtggagcagagctaccctggcaggagccactactactgggacgaggactcagggctgttgttcctgaagctgaaagctcagaacgagagagagaagtttgctttctgctccatgaaaggctgtgagaggataaagattaaagctctgattccaaagaacgcaggcgtcagtgactgcacagccacagcttaccccaagttcaccgagagggctgtcgtagacgtgccgatgcccaagaagctctttggttctcagctgaaaacaaaggaccatttcttggaggtgaagatggagagttccaagcagcacttcttccacctctggaacgacttcgcttacattgaagtggatgggaagaagtaccccagttcggaggatggcatccaggtggtggtgattgacgggaaccaagggcgcgtggtgagccacacgagcttcaggaactccattctgcaaggcataccatggcagcttttcaactatgtggcgaccatccctgacaattccatagtgcttatggcatcaaagggaagatacgtctccagaggcccatggaccagagtgctggaaaagcttggggcagacaggggtctcaagttgaaagagcaaatggcattcgttggcttcaaaggcagcttccggcccatctgggtgacactggacactgaggatcacaaagccaaaatcttccaagttgtgcccatccctgtggtgaagaagaagaagttgtgaggacagctgccgcccggtgccacctcgtggtagactatg

SEQ ID NO: 5 - DNA, 2810 bp, Homo sapiens
gccccctggcccgagccgcgcccgggtctgtgagtagagccgcccgggcaccgagcgctggtcgccgctctccttccgttatatcaacatgccccctttcctgttgctggaagccgtctgtgttttcctgttttccagagtgcccccatctctccctctccaggaagtccatgtaagcaaagaaaccatcgggaagatttcagctgccagcaaaatgatgtggtgctcggctgcagtggacatcatgtttctgttagatgggtctaacagcgtcgggaaagggagctttgaaaggtccaagcactttgccatcacagtctgtgacggtctggacatcagccccgagagggtcagagtgggagcattccagttcagttccactcctcatctggaattccccttggattcattttcaacccaacaggaagtgaaggcaagaatcaagaggatggttttcaaaggagggcgcacggagacggaacttgctctgaaataccttctgcacagagggttgcctggaggcagaaatgcttctgtgccccagatcctcatcatcgtcactgatgggaagtcccagggggatgtggcactgccatccaagcagctgaaggaaaggggtgtcactgtgtttgctgtgggggtcaggtttcccaggtgggaggagctgcatgcactggccagcgagcctagagggcagcacgtgctgttggctgagcaggtggaggatgccaccaacggcctcttcagcaccctcagcagctcggccatctgctccagcgccacgccagactgcagggtcgaggctcacccctgtgagcacaggacgctggagatggtccgggagttcgctggcaatgccccatgctggagaggatcgcggcggacccttgcggtgctggctgcacactgtcccttctacagctggaagagagtgttcctaacccaccctgccacctgctacaggaccacctgcccaggcccctgtgactcgcagccctgccagaatggaggcacatgtgttccagaaggactggacggctaccagtgcctctgcccgctggcctttggaggggaggctaactgtgccctgaagctgagcctggaatgcagggtcgacctcctcttcctgctggacagctctgcgggcaccactctggacggcttcctgcgggccaaagtcttcgtgaagcggtttgtgcgggccgtgctgagcgaggactctcgggcccgagtgggtgtggccacatacagcagggagctgctggtggcggtgcctgtgggggagtaccaggatgtgcctgacctggtctggagcctcgatggcattcccttccgtggtggccccaccctgacgggcagtgccttgcggcaggcggcagagcgtggcttcgggagcgccaccaggacaggccaggaccggccacgtagagtggtggttttgctcactgagtcacactccgaggatgaggttgcgggcccagcgcgtcacgcaagggcgcgagagctgctcctgctgggtgtaggcagtgaggccgtgcgggcagagctggaggagatcacaggcagcccaaagcatgtgatggtctactcggatcctcaggatctgttcaaccaaatccctgagctgcaggggaagctgtgcagccggcagcggccagggtgccggacacaagccctggacctcgtcttcatgttggacacctctgcctcagtagggcccgagaattttgctcagatgcagagctttgtgagaagctgtgccctccagtttgaggtgaaccctgacgtgacacaggtcggcctggtggtgtatggcagccaggtgcagactgccttcgggctggacaccaaacccacccgggctgcgatgctgcgggccattagccaggccccctacctaggtggggtgggctcagccggcaccgccctgctgcacatctatgacaaagtgatgaccgtccagaggggtgcccggcctggtgtccccaaagctgtggtggtgctcacaggcgggagaggcgcagaggatgcagccgttcctgcccagaagctgaggaacaatggcatctctgtcttggtcgtgggcgtggggcctgtcctaagtgagggtctgcggaggcttgcaggtccccgggattccctgatccacgtggcagcttacgccgacctgcggtaccaccaggacgtgctcattgagtggctgtgtggagaagccaagcagccagtcaacctctgcaaacccagcccgtgcatgaatgagggcagctgcgtcctgcagaatgggagctaccgctgcaagtgtcgggatggctgggagggcccccactgcgagaaccgattcttgagacgcccctgaggcacatggctcccgtgcaggagggcagcagccgtacccctcccagcaactacagagaaggcctgggcactgaaatggtgcctaccttctggaatgtctgtgccccaggtccttagaatgtctgcttcccgccgtggccaggaccactattctcactgagggaggaggatgtcccaactgcagccatgctgcttagagacaagaaagcagctgatgtcacccacaaacgatgttgttgaaaagttttgatgtgtaagtaaatacccactttctgtacctgctgtgccttgttgaggctatgtcatctgccacctttcccttgaggataaacaaggggtcctgaagacttaaatttagcggcctgacgttcctttgcacacaatcaatgctcgccagaatgttgttgacacagtaatgcccagcagaggcctttactagagcatcctttggacgg

SEQ ID NO: 6 - DNA, 1524 bp, Homo sapiens
gcagagcacagcatcgtcgggaccagactcgtctcaggccagttgcagccttctcagccaaacgccgaccaaggaaaactcactaccatgagaattgcagtgatttgcttttgcctcctaggcatcacctgtgccataccagttaaacaggctgattctggaagttctgaggaaaagcagctttacaacaaatacccagatgctgtggccacatggctaaaccctgacccatctcagaagcagaatctcctagccccacagacccttccaagtaagtccaacgaaagccatgaccacatggatgatatggatgatgaagatgatgatgaccatgtggacagccaggactccattgactcgaacgactctgatgatgtagatgacactgatgattctcaccagtctgatgagtctcaccattctgatgaatctgatgaactggtcactgattttcccacggacctgccagcaaccgaagttttcactccagttgtccccacagtagacacatatgatggccgaggtgatagtgtggtttatggactgaggtcaaaatctaagaagtttcgcagacctgacatccagtaccctgatgctacagacgaggacatcacctcacacatggaaagcgaggagttgaatggtgcatacaaggccatccccgttgcccaggacctgaacgcgccttctgattgggacagccgtgggaaggacagttatgaaacgagtcagctggatgaccagagtgctgaaacccacagccacaagcagtccagattatataagcggaaagccaatgatgagagcaatgagcattccgatgtgattgatagtcaggaactttccaaagtcagccgtgaattccacagccatgaatttcacagccatgaagatatgctggttgtagaccccaaaagtaaggaagaagataaacacctgaaatttcgtatttctcatgaattagatagtgcatcttctgaggtcaattaaaaggagaaaaaatacaatttctcactttgcatttagtcaaaagaaaaaatgctttatagcaaaatgaaagagaacatgaaatgcttctttctcagtttattggttgaatgtgtatctatttgagtctggaaataactaatgtgtttgataattagtttagtttgtggcttcatggaaactccctgtaaactaaaagcttcagggttatgtctatgttcattctatagaagaaatgcaaactatcactgtattttaatatttgttattctctcatgaatagaaatttatgtagaagcaaacaaaatacttttacccacttaaaaagagaatataacattttatgtcactataatcttttgttttttaagttagtgtatattttgttgtgattatctttttgtggtgtgaataaatcttttatcttgaatgtaataagaatttggtggtgtcaattgcttatttgttttcccacggttgtccagcaattaataaaacataaccttttttactgcctaaaaaaaaaaaaaaaaaaaa

SEQ ID NO: 7 - DNA, 3205 bp, Homo sapiens
aaaggggcaagagctgagcggaacaccggcccgccgtcgcggcagctgcttcacccctctctctgcagccatggggctccctcgtggacctctcgcgtctctcctccttctccaggtttgctggctgcagtgcgcggcctccgagccgtgccgggcggtcttcagggaggctgaagtgaccttggaggcgggaggcgcggagcaggagcccggccaggcgctggggaaagtattcatgggctgccctgggcaagagccagctctgtttagcactgataatgatgacttcactgtgcggaatggcgagacagtccaggaaagaaggtcactgaaggaaaggaatccattgaagatcttcccatccaaacgtatcttacgaagacacaagagagattgggtggttgctccaatatctgtccctgaaaatggcaagggtcccttcccccagagactgaatcagctcaagtctaataaagatagagacaccaagattttctacagcatcacggggccgggggcagacagcccccctgagggtgtcttcgctgtagagaaggagacaggctggttgttgttgaataagccactggaccgggaggagattgccaagtatgagctctttggccacgctgtgtcagagaatggtgcctcagtggaggaccccatgaacatctccatcatcgtgaccgaccagaatgaccacaagcccaagtttacccaggacaccttccgagggagtgtcttagagggagtcctaccaggtacttctgtgatgcaggtgacagccacggatgaggatgatgccatctacacctacaatggggtggttgcttactccatccatagccaagaaccaaaggacccacacgacctcatgttcaccattcaccggagcacaggcaccatcagcgtcatctccagtggcctggaccgggaaaaagtccctgagtacacactgaccatccaggccacagacatggatggggacggctccaccaccacggcagtggcagtagtggagatccttgatgccaatgacaatgctcccatgtttgacccccagaagtacgaggcccatgtgcctgagaatgcagtgggccatgaggtgcagaggctgacggtcactgatctggacgcccccaactcaccagcgtggcgtgccacctaccttatcatgggcggtgacgacggggaccattttaccatcaccacccaccctgagagcaaccagggcatcctgacaaccaggaagggtttggattttgaggccaaaaaccagcacaccctgtacgttgaagtgaccaacgaggccccttttgtgctgaagctcccaacctccacagccaccatagtggtccacgtggaggatgtgaatgaggcacctgtgtttgtcccaccctccaaagtcgttgaggtccaggagggcatccccactggggagcctgtgtgtgtctacactgcagaagaccctgacaaggagaatcaaaagatcagctaccgcatcctgagagacccagcagggtggctagccatggacccagacagtgggcaggtcacagctgtgggcaccctcgaccgtgaggatgagcagtttgtgaggaacaacatctatgaagtcatggtcttggccatggacaatggaagccctcccaccactggcacgggaacccttctgctaacactgattgatgtcaatgaccatggcccagtccctgagccccgtcagatcaccatctgcaaccaaagccctgtgcgccaggtgctgaacatcacggacaaggacctgtctccccacacctcccctttccaggcccagctcacagatgactcagacatctactggacggcagaggtcaacgaggaaggtgacacagtggtcttgtccctgaagaagttcctgaagcaggatacatatgacgtgcacctttctctgtctgaccatggcaacaaagagcagctgacggtgatcagggccactgtgtgcgactgccatggccatgtcgaaacctgccctggaccctggaagggaggtttcatcctccctgtgctgggggctgtcctggctctgctgttcctcctgctggtgctgcttttgttggtgagaaagaagcggaagatcaaggagcccctcctactcccagaagatgacacccgtgacaacgtcttctactatggcgaagaggggggtggcgaagaggaccaggactatgacatcacccagctccaccgaggtctggaggccaggccggaggtggttctccgcaatgacgtggcaccaaccatcatcccgacacccatgtaccgtcctcggccagccaacccagatgaaatcggcaactttataattgagaacctgaaggcggctaacacagaccccacagccccgccctacgacaccctcttggtgttcgactatgagggcagcggctccgacgccgcgtccctgagctccctcacctcctccgcctccgaccaagaccaagattacgattatctgaacgagtggggcagccgcttcaagaagctggcagacatgtacggtggcggggaggacgactaggcggcctgcctgcagggctggggaccaaacgtcaggccacagagcatctccaaggggtctcagttcccccttcagctgaggacttcggagcttgtcaggaagtggccgtagcaacttggcggagacaggctatgagtctgacgttagagtggttgcttccttagcctttcaggatggaggaatgtgggcagtttgacttcagcactgaaaacctctccacctgggccagggttgcctcagaggccaagtttccagaagcctcttacctgccgtaaaatgctcaaccctgtgtcctgggcctgggcctgctgtgactgacctacagtggactttctctctggaatggaaccttcttaggcctcctggtgcaacttaatttttttttttaatgctatcttcaaaacgttagagaaagttcttcaaaagtgcagcccagagctgctgggcccactggccgtcctgcatttctggtttccagaccccaatgcctcccattcggatggatctctgcgtttttatactgagtgtgcctaggttgccccttattttttattttccctgttgcgttgctatagatgaagggtgaggacaatcgtgtatatgtactagaacttttttattaaagaaacttttcccagaaaaaaa

SEQ ID NO: 8 - DNA, 2603 bp, Homo sapiens
atgaagcacctgaagcggtggtggtcggccggcggcggcctcctgcacctcaccctcctgctgagcttggcggggctccgcgtagacctagatctttacctgctgctgccgccgcccaccctgctgcaggacgagctgctgttcctgggcggcccggccagctccgcctacgcgctcagccccttctcggcctcgggagggtgggggcgcgcgggccacttgcaccccaagggccgggagctggaccctgccgcgccgcccgagggccagctgctccgggaggtgcgcgcgctcggggtccccttcgtccctcgcaccagcgtggatgcatggctggtgcacagcgtggctgccgggagcgcggacgaggcccacgggctgctcggcgccgccgccgcctcgtccaccggaggagccggcgccagcgtggacggcggcagccaggctgtgcaggggggcggcggggacccccgagcggctcggagtggccccttggacgccggggaagaggagaaggcacccgcggaaccgacggctcaggtgccggacgctggcggatgtgcgagcgaggagaatggggtactaagagaaaagcacgaagctgtggatcatagttcccagcatgaggaaaatgaagaaagggtgtcagcccagaaggagaactcacttcagcagaatgatgatgatgaaaacaaaatagcagagaaacctgactgggaggcagaaaagaccactgaatctagaaatgagagacatctgaatgggacagatacttctttctctctggaagacttattccagttgctttcatcacagcctgaaaattcactggagggcatctcattgggagatattcctcttccaggcagtatcagtgatggcatgaattcttcagcacattatcatgtaaacttcagccaggctataagtcaggatgtgaatcttcatgaggccatcttgctttgtcccaacaatacatttagaagagatccaacagcaaggacttcacagtcacaagaaccatttctgcagttaaattctcataccaccaatcctgagcaaacccttcctggaactaatttgacaggatttctttcaccggttgacaatcatatgaggaatctaacaagccaagacctactgtatgaccttgacataaatatatttgatgagataaacttaatgtcattggccacagaagacaactttgatccaatcgatgtttctcagctttttgatgaaccagattctgattctggcctttctttagattcaagtcacaataatacctctgtcatcaagtctaattcctctcactctgtgtgtgatgaaggtgctataggttattgcactgaccatgaatctagttcccatcatgacttagaaggtgctgtaggtggctactacccagaacccagtaagctttgtcacttggatcaaagtgattctgatttccatggagatcttacatttcaacacgtatttcataaccacacttaccacttacagccaactgcaccagaatctacttctgaaccttttccgtggcctgggaagtcacagaagataaggagtagataccttgaagacacagatagaaacttgagccgtgatgaacagcgtgctaaagctttgcatatccctttttctgtagatgaaattgtcggcatgcctgttgattctttcaatagcatgttaagtagatattatctgacagacctacaagtctcacttatccgtgacatcagacgaagagggaaaaataaagttgctgcgcagaactgtcgtaaacgcaaattggacataattttgaatttagaagatgatgtatgtaacttgcaagcaaagaaggaaactcttaagagagagcaagcacaatgtaacaaagctattaacataatgaaacagaaactgcatgacctttatcatgatatttttagtagattaagagatgaccaaggtaggccagtcaatcccaaccactatgctctccagtgtacccatgatggaagtatcttgatagtacccaaagaactggtggcctcaggccacaaaaaggaaacccaaaagggaaagagaaagtgagaagaaactgaagatggactctattatgtgaagtagtaatgttcagaaactgattatttggatcagaaaccattgaaactgcttcaagaattgtatctttaagtactgctacttgaataactcagttaacgctgttttgaagcttacatggacaaatgtttaggacttcaagatcacacttgtgggcaatctgggggagccacaacttttcatgaagtgcattgtatacaaaattcatagttatgtccaaagaataggttaacatgaaaacccagtaagactttccatcttggcagccatcctttttaagagtaagttggttacttcaaaaagagcaaacactggggatcaaattattttaagaggtatttcagttttaaatgcaaaatagccttattttcatttagtttgttagcactatagtgagcttttcaaacactattttaatctttatatttaacttataaattttgctttctatggaaataaattttgtatttgtattaaaaaaaaaaaaa

SEQ ID NO: 9 - DNA, 1209 bp, Homo sapiens
  misc_feature 1161: n = A,T,C or G
atgaagttggaggtgttcgtccctcgcgcggcccacggggacaagcagggcagtgacctggagggcgcgggcggcagcgacgcgccgtccccgctgtcggcggcgggagacgactccctgggctcagatggggactgcgcggccaagccgtccgcgggcggcggcgccagagatacgcagggcgacggcgaacagagtgcgggaggcgggccgggcgcggaggaggcgatcccggcagcagctgctgcagcggtggtggcggagggcgcggaggccggggcggcggggccaggcgcgggcggcgcggggagcggcgagggtgcacgcagcaagccatatacgcggcggcccaagcccccctactcgtacatcgcgctcatcgccatggccatccgcgactcggcgggcgggcgcttgacgctggcggagatcaacgagtacctcatgggcaagttcccctttttccgcggcagctacacgggctggcgcaactccgtgcgccacaacctttcgctcaacgactgcttcgtcaaggtgctgcgcgacccctcgcggccctggggcaaggacaactactggatgctcaaccccaacagcgagtacaccttcgccgacggggtcttccgccgccgccgcaagcgcctcagccaccgcgcgccggtccccgcgcccgggctgcggcccgaggaggccccgggcctccccgccgccccgccgcccgcgcccgccgccccggcctcgccccgcatgcgctcgcccgcccgccaggaggagcgcgccagccccgcgggcaagttctccagctccttcgccatcgacagcatcctgcgcaagcccttccgcagccgtcgcctcagggacacggcccccgggacgacgcttcagtggggcgccgcgccctgcccgccgctgcccgcgttccccgcgctcctccccgcggcgccctgcagggccctgctgccgctctgcgcgtacggcgcgggcgagccggcgcggctgggcgcgcgcgaggccgaggtgccaccgaccgcgccgcccctcctgcttgcacctctcccggcggcggcccccgccaagccactccgaggcccggcggccggcggcgcgcacctgtactgccccctgcggctgcccgcagccctgcaggcggccttagtccgncgtcctggcccgcacctgtcgtacccggtggagacgctcctagcttga

SEQ ID NO: 10 - DNA, 1474 bp, Homo sapiens
ggcagatgaaatataagattcatcaaccacatttgacagcccatggcaggtttcctgttttccatcgtccctctgcaggtcacagacacacagagcccagccgtggcaggctcagccggggtccggggctgctaacaacggctacattcctcccccagggccaagggaaatcctgagcgcaggccagggttgtttggttttgaggtgtgctgggatgaaaggcaccctggaagtggaaggttcggtcattcattaattaattacatctataattgagggtttgttcttaagagcgagtcctttgaaagtactttccttcaaacagtgactgccacaaaggcatcagatattcaccaccttctcggctgcctcagcacagcaagctttattctgggacctgagatcctgttctgagctggctttcccttctccaggctcgctcaccctccctttagagatagtggatggtaagatgaccaatgctcagattattcttctcattgacaatgccaggatggcagtggatgacttcaacctcaagaaatggagaagcatcatgtgccaagtgacttcaatgtcaatgtgaaggtggatacaggtcccagggaagatctgattaaggtcctggaggatatgagacaagaatatgagcttataataaagaagaagcatcgagacttggacacttggtataaagaacagtctgcagccatgtcccaggaggcagccagtccagccactgtgcagagcagacaaggtgacatccacgaactgaagcgcacattccaggccctggagattgacctgcaggcacagtacagcacgaaatctgctttggaaaacatgttatccgagacccagtctcggtactcctgcaagctccaggacatgcaagagatcatctcccactatgaggaggaactgacgcagctacgccacgaactggagcggcagaacaatgaataccaagtgctgctgggcatcaaaacccacctggagaaggaaatcaccacgtaccgacggctcctggagggagagagtgaagggacacgggaagaatcaaagtcgagcatgaaagtgtctgcaactccaaagatcaaggccataacccaggagaccatcaacggaagattagttctttgtcaagtgaatgaaatccaaaagcacgcatgagaccaatgaaagtttccgcctgttgtaaaatctattttcccccaaggaaagtccttgcacagacaccagtgagtgagttctaaaagatacccttggaattatcagactcagaaacttttattttttttttctgtaacagtctcaccagacttctcataatgctcttaatatattgcacttttctaatcaaagtgcgagtttatgagggtaaagctctactttcctactgcagccttcagattctcatcattttgcatctattttgtagccaataaaactccgcactagcaaaaaaaaaaaa

SEQ ID NO: 11 - DNA, 411 bp, Homo sapiens
ttttttttttaaaaaaagaggcttggtaagtttttgatgcttagttgacttttagcattatccagcatttgtattatgaaccagtgagtactgtaatttttctttccctttcagaaagactcaaagggaacatataaatgtttcctatttttaatgtggcaatagtgtagctaacactggtacagacggaataaacacacctctaatattctcctgaagatttggtgatccagtttcaaataaggtatgggaaaaacagatgttttcattatcgccacttaatccttacttccgattataattatacatgtttggctgtaataactatactaaagcatgcttgtgaaagtagacttctacaaggacagaaaacccacaacaacaaagatcgatcacgaaagacaaggcata

SEQ ID NO: 12 - DNA, 2336 bp, Homo sapiens
cttttcttccgcacggttggaggaggtcggctggttatcgggagttggagggctgaggtcgggagggtggtgtgtacagagctctaggactcacgcaccaggccagtcgcggattttgggccgaggcctgggttacaagcagcaagtgcgcggttggggccactgcgaggccgttttagaaaactgtttaaaacaaagagcaattgatggataaatcaggaatagattctcttgaccatgtgacatctgatgctgtggaacttgcaaatcgaagtgataactcttctgatagcagcttatttaaaactcagtgtatcccttactcacctaaaggggagaaaagaaaccccattcgaaaatttgttcgtacacctgaaagtgttcacgcaagtgattcatcaagtgactcatcttttgaaccaataccattgactataaaagctatttttgaaagattcaagaacaggaaaaagagatataaaaaaaagaaaaagaggaggtaccagccaacaggaagaccacggggaagaccagaaggaaggagaaatcctatatactcactaatagataagaagaaacaatttagaagcagaggatctggcttcccatttttagaatcagagaatgaaaaaaacgcaccttggagaaaaattttaacgtttgagcaagctgttgcaagaggattttttaactatattgaaaagctgaagtatgaacaccacctgaaagaatcattgaagcaaatgaatgttggtgaagatttagaaaatgaagattttgacagtcgtagatacaaatttttggatgatgatggatccatttctcctattgaggagtcaacagcagaggatgaggatgcaacacatcttgaagataacgaatgtgatatcaaattggcaggggatagtttcatagtaagttctgaattccctgtaagactgagtgtatacttagaagaagaggatattactgaagaagctgctttgtctaaaaagagagctacaaaagccaaaaatactggacagagaggcctgaaaatgtgacaggatcatgaatgtcaaaggcttttatcttgagaacatggtgtctggagttaaaggtattggcatactccacacatctgtaccattcttgagtgatcgcttaggaatgaatgtgatttgaactcattcatgttgagagggtgtcaaattgagaaccaggtagatccccaccacctacagtaaaaaggaccctaaagtaaattggttgaagaaattagatcccaaagattcttggtgaattttgaagtcttcatcagtatatccatattaaaacgagatgacagaagccaaagtaattatggcaagtaatggtttttatcttaactataagttatttgctcaagggtgtaatggtcattaccaaggcttttagaatgcagtttctcatttgctgtggacatgaccataaaaaaaaatttcccagtaggttttctatctgctacgttgctagcaatcagcttattgggaacagttgattaactgtaatagaaatgcaatacaaataaaatgtgaaccacatgtgatttttctttaaaatcagtgagatttgaaaattctcctagatctcttgaatcatgcaaatttgctttgcctttatattgtaacccttgtgggttgctaataaccaagcagtttgtagtagagttaactcaggctcgttctagggactcattcatgttcactcactgtacactcatctctggaaatgtaaaatttacttttatactattgttatgtagggctgacaggacaactggatcagtttcattaaaaaggtatgtatgcattagaaaagacatttgtatgggtcatttcaaagagggcttatgaggctgtgaaacccagagctcttaacgctgtgaccaaagatggaagttctctataggaagccatagcactcctaatgtttggtgctatgttttcctgaggagatataaaacgtaataatccatgattgttgccatgtgagagttttaaaggttaatcaaaatttctcttcttcagggcaaacttgaagataaatcttttgactccagctctttagaggatctaaagtgaccttgatggacagtggaagaaatcacaacatggaattcctcgaataacaatttattgactttaaataattttgtctaatgctacatatacacaattaaaaaacctttacactatttctagaaagtcagcatgtatttttggctcgaagtttctctagtgttttctgtggaaggaataaaaatttgagtttcaaaaaaaaaaaaaaaaaaaaaaaaaaaaaaaaaaaaaaaaaaa

SEQ ID NO: 13 - protein, 1361 aa, Homo sapiens
MGAAGRQDFLFKAMLTISWLTLTCFPGATSTVAAGCPDQSPELQPWNPGHDQDHHVHIGQGKTLLLTSSATVYSIHISEGGKLVIKDHDEPIVLRTRHILIDNGGELHAGSALCPFQGNFTIILYGRADEGIQPDPYYGLKYIGVGKGGALELHGQKKLSWTFLNKTLHPGGMAEGGYFFERSWGHRGVIVHVIDPKSGTVIHSDRFDTYRSKKESERLVQYLNAVPDGRILSVAVNDEGSRNLDDMARKAMTKLGSKHFLHLGFRHPWSFLTVKGNPSSSVEDHIEYHGHRGSAAARVFKLFQTEHGEYFNVSLSSEWVQDVEWTEWFDHDKVSQTKGGEKISDLWKAHPGKICNRPIDIQATTMDGVNLSTEVVYKKGQDYRFACYDRGRACRSYRVRFLCGKPVRPKLTVTIDTNVNSTILNLEDNVQSWKPGDTLVIASTDYSMYQAEEFQVLPCRSCAPNQVKVAGKPMYLHIGEEIDGVDMRAEVGLLSRNIIVMGEMEDKCYPYRNHICNFFDFDTFGGHIKFALGFKAAHLEGTELKHMGQQLVGQYPIHFHLAGDVDERGGYDPPTYIRDLSIHHTFSRCVTVHGSNGLLIKDVVGYNSLGHCFFTEDGPEERNTFDHCLGLLVKSGTLLPSDRDSKMCKMITEDSYPGYIPKPRQDCNAVSTFWMANPNNNLINCAAAGSEETGFWFIFHHVPTGPSVGMYSPGYSEHIPLGKFYNNRAHSNYRAGMIIDNGVKTTEASAKDKRPFLSIISARYSPHQDADPLKPREPAIIRHFIAYKNQDHGAWLRGGDVWLDSCRFADNGIGLTLASGGTFPYDDGSKQEIKNSLFVGESGNVGTEMMDNRIWGPGGLDHSGRTLPIGQNFPIRGIQLYDGPINIQNCTFRKFVALEGRHTSALAFRLNNAWQSCPHNNVTGIAFEDVPITSRVFFGEPGPWFNQLDMDGDKTSVFHDVDGSVSEYPGSYLTKNDNWLVRHPDCINVPDWRGAICSGCYAQMYIQAYKTSNLRMKIIKNDFPSHPLYLEGALTRSTHYQQYQPVVTLQKGYTIHWDQTAPAELAIWLINFNKGDWIRVGLCYPRGTTFSILSDVHNRLLKQTSKTGVFVRTLQMDKVEQSYPGRSHYYWDEDSGLLFLKLKAQNEREKFAFCSMKGCERIKIKALIPKNAGVSDCTATAYPKFTERAVVDVPMPKKLFGSQLKTKDHFLEVKMESSKQHFFHLWNDFAYIEVDGKKYPSSEDGIQVVVIDGNQGRVVSHTSFRNSILQGIPWQLFNYVATIPDNSIVLMASKGRYVSRGPWTRVLEKLGADRGLKLKEQMAFVGFKGSFRPIWVTLDTEDHKAKIFQVVPIPVVKKKKL

SEQ ID NO: 14 - protein, 755 aa, Homo sapiens
MPPFLLLEAVCVFLFSRVPPSLPLQEVHVSKETIGKISAASKMMWCSAAVDIMFLLDGSNSVGKGSFERSKHFAITVCDGLDISPERVRVGAFQFSSTPHLEFPLDSFSTQQEVKARIKRMVFKGGRTETELALKYLLHRGLPGGRNASVPQILIIVTDGKSQGDVALPSKQLKERGVTVFAVGVRFPRWEELHALASEPRGQHVLLAEQVEDATNGLFSTLSSSAICSSATPDCRVEAHPCEHRTLEMVREFAGNAPCWRGSRRTLAVLAAHCPFYSWKRVFLTHPATCYRTTCPGPCDSQPCQNGGTCVPEGLDGYQCLCPLAFGGEANCALKLSLECRVDLLFLLDSSAGTTLDGFLRAKVFVKRFVRAVLSEDSRARVGVATYSRELLVAVPVGEYQDVPDLVWSLDGIPFRGGPTLTGSALRQAAERGFGSATRTGQDRPRRVVVLLTESHSEDEVAGPARHARARELLLLGVGSEAVRAELEEITGSPKHVMVYSDPQDLFNQIPELQGKLCSRQRPGCRTQALDLVFMLDTSASVGPENFAQMQSFVRSCALQFEVNPDVTQVGLVVYGSQVQTAFGLDTKPTRAAMLRAISQAPYLGGVGSAGTALLHIYDKVMTVQRGARPGVPKAVVVLTGGRGAEDAAVPAQKLRNNGISVLVVGVGPVLSEGLRRLAGPRDSLIHVAAYADLRYHQDVLIEWLCGEAKQPVNLCKPSPCMNEGSCVLQNGSYRCKCRDGWEGPHCENRFLRRP

SEQ ID NO: 15 - protein, 300 aa, Homo sapiens
MRIAVICFCLLGITCAIPVKQADSGSSEEKQLYNKYPDAVATWLNPDPSQKQNLLAPQTLPSKSNESHDHMDDMDDEDDDDHVDSQDSIDSNDSDDVDDTDDSHQSDESHHSDESDELVTDFPTDLPATEVFTPVVPTVDTYDGRGDSVVYGLRSKSKKFRRPDIQYPDATDEDITSHMESEELNGAYKAIPVAQDLNAPSDWDSRGKDSYETSQLDDQSAETHSHKQSRLYKRKANDESNEHSDVIDSQELSKVSREFHSHEFHSHEDMLVVDPKSKEEDKHLKFRISHELDSASSEVN

SEQ ID NO: 16 - protein, 829 aa, Homo sapiens
MGLPRGPLASLLLLQVCWLQCAASEPCRAVFREAEVTLEAGGAEQEPGQALGKVFMGCPGQEPALFSTDNDDFTVRNGETVQERRSLKERNPLKIFPSKRILRRHKRDWVVAPISVPENGKGPFPQRLNQLKSNKDRDTKIFYSITGPGADSPPEGVFAVEKETGWLLLNKPLDREEIAKYELFGHAVSENGASVEDPMNISIIVTDQNDHKPKFTQDTFRGSVLEGVLPGTSVMQVTATDEDDAIYTYNGVVAYSIHSQEPKDPHDLMFTIHRSTGTISVISSGLDREKVPEYTLTIQATDMDGDGSTTTAVAVVEILDANDNAPMFDPQKYEAHVPENAVGHEVQRLTVTDLDAPNSPAWRATYLIMGGDDGDHFTITTHPESNQGILTTRKGLDFEAKNQHTLYVEVTNEAPFVLKLPTSTATIVVHVEDVNEAPVFVPPSKVVEVQEGIPTGEPVCVYTAEDPDKENQKISYRILRDPAGWLAMDPDSGQVTAVGTLDREDEQFVRNNIYEVMVLAMDNGSPPTTGTGTLLLTLIDVNDHGPVPEPRQITICNQSPVRQVLNITDKDLSPHTSPFQAQLTDDSDIYWTAEVNEEGDTVVLSLKKFLKQDTYDVHLSLSDHGNKEQLTVIRATVCDCHGHVETCPGPWKGGFILPVLGAVLALLFLLLVLLLLVRKKRKIKEPLLLPEDDTRDNVFYYGEEGGGEEDQDYDITQLHRGLEARPEVVLRNDVAPTIIPTPMYRPRPANPDEIGNFIIENLKAANTDPTAPPYDTLLVFDYEGSGSDAASLSSLTSSASDQDQDYDYLNEWGSRFKKLADMYGGGEDD

SEQ ID NO: 17 - protein, 694 aa, Homo sapiens
MKHLKRWWSAGGGLLHLTLLLSLAGLRVDLDLYLLLPPPTLLQDELLFLGGPASSAYALSPFSASGGWGRAGHLHPKGRELDPAAPPEGQLLREVRALGVPFVPRTSVDAWLVHSVAAGSADEAHGLLGAAAASSTGGAGASVDGGSQAVQGGGGDPRAARSGPLDAGEEEKAPAEPTAQVPDAGGCASEENGVLREKHEAVDHSSQHEENEERVSAQKENSLQQNDDDENKIAEKPDWEAEKTTESRNERHLNGTDTSFSLEDLFQLLSSQPENSLEGISLGDIPLPGSISDGMNSSAHYHVNFSQAISQDVNLHEAILLCPNNTFRRDPTARTSQSQEPFLQLNSHTTNPEQTLPGTNLTGFLSPVDNHMRNLTSQDLLYDLDINIFDEINLMSLATEDNFDPIDVSQLFDEPDSDSGLSLDSSHNNTSVIKSNSSHSVCDEGAIGYCTDHESSSHHDLEGAVGGYYPEPSKLCHLDQSDSDFHGDLTFQHVFHNHTYHLQPTAPESTSEPFPWPGKSQKIRSRYLEDTDRNLSRDEQRAKALHIPFSVDEIVGMPVDSFNSMLSRYYLTDLQVSLIRDIRRRGKNKVAAQNCRKRKLDIILNLEDDVCNLQAKKETLKREQAQCNKAINIMKQKLHDLYHDIFSRLRDDQGRPVNPNHYALQCTHDGSILIVPKELVASGHKKETQKGKRK

SEQ ID NO: 18 - protein, 402 aa, Homo sapiens
MKLEVFVPRAAHGDKQGSDLEGAGGSDAPSPLSAAGDDSLGSDGDCAAKPSAGGGARDTQGDGEQSAGGGPGAEEAIPAAAAAAVVAEGAEAGAAGPGAGGAGSGEGARSKPYTRRPKPPYSYIALIAMAIRDSAGGRLTLAEINEYLMGKFPFFRGSYTGWRNSVRHNLSLNDCFVKVLRDPSRPWGKDNYWMLNPNSEYTFADGVFRRRRKRLSHRAPVPAPGLRPEEAPGLPAAPPPAPAAPASPRMRSPARQEERASPAGKFSSSFAIDSILRKPFRSRRLRDTAPGTTLQWGAAPCPPLPAFPALLPAAPCRALLPLCAYGAGEPARLGAREAEVPPTAPPLLLAPLPAAAPAKPLRGPAAGGAHLYCPLRLPAALQAALVRRPGPHLSYPVETLLA

SEQ ID NO: 19 - protein, 209 aa, Homo sapiens
MEKHHVPSDFNVNVKVDTGPREDLIKVLEDMRQEYELIIKKKHRDLDTWYKEQSAAMSQEAASPATVQSRQGDIHELKRTFQALEIDLQAQYSTKSALENMLSETQSRYSCKLQDMQEIISHYEEELTQLRHELERQNNEYQVLLGIKTHLEKEITTYRRLLEGESEGTREESKSSMKVSATPKIKAITQETINGRLVLCQVNEIQKHA

SEQ ID NO: 20 - protein, 278 aa, Homo sapiens
MDKSGIDSLDHVTSDAVELANRSDNSSDSSLFKTQCIPYSPKGEKRNPIRKFVRTPESVHASDSSSDSSFEPIPLTIKAIFERFKNRKKRYKKKKKRRYQPTGRPRGRPEGRRNPIYSLIDKKKQFRSRGSGFPFLESENEKNAPWRKILTFEQAVARGFFNYIEKLKYEHHLKESLKQMNVGEDLENEDFDSRRYKFLDDDGSISPIEESTAEDEDATHLEDNECDIKLAGDSFIVSSEFPVRLSVYLEEEDITEEAALSKKRATKAKNTGQRGLKM

SEQ ID NO: 21 - protein, 488 aa, Homo sapiens
AVLAAHCPFYSWKRVFLTHPATCYRTTCPGPCDSQPCQNGGTCVPEGLDGYQCLCPLAFGGEANCALKLSLECRVDLLFLLDSSAGTTLDGFLRAKVFVKRFVRAVLSEDSRARVGVATYSRELLVAVPVGEYQDVPDLVWSLDGIPFRGGPTLTGSALRQAAERGFGSATRTGQDRPRRVVVLLTESHSEDEVAGPARHARARELLLLGVGSEAVRAELEEITGSPKHVMVYSDPQDLFNQIPELQGKLCSRQRPGCRTQALDLVFMLDTSASVGPENFAQMQSFVRSCALQFEVNPDVTQVGLVVYGSQVQTAFGLDTKPTRAAMLRAISQAPYLGGVGSAGTALLHIYDKVMTVQRGARPGVPKAVVVLTGGRGAEDAAVPAQKLRNNGISVLVVGVGPVLSEGLRRLAGPRDSLIHVAAYADLRYHQDVLIEWLCGEAKQPVNLCKPSPCMNEGSCVLQNGSYRCKCRDGWEGPHCENRFLRRP

SEQ ID NO: 22 - protein, 13 aa, Homo sapiens
AVLAAHCPFYSWK

(19) United States
(12) Patent Application Publication
(10) Pub. No.: US 2005/0233353 A1
(43) Pub. Date: Oct. 20, 2005

(54) Methods and compositions for categorizing patients

(76) Inventor: Sanford D. Markowitz, Pepper Pike, OH (US)

Correspondence Address: FISH & NEAVE IP GROUP;ROPES & GRAY LLP, ONE INTERNATIONAL PLACE, BOSTON, MA, 02110-2624, US

(21) Appl. No.: 11/041,788

(22) Filed: Jan. 21, 2005

Related U.S. Application Data
(63) Continuation-in-part of application No. 10/649,591, filed on Aug. 26, 2003.
Application No. 10/649,591 is a continuation-in-part of application No. 10/274,177, filed on Oct. 18, 2002.
Application No. 10/274,177 is a continuation-in-part of application No. 10/229,345, filed on Aug. 26, 2002.
(63) Continuation-in-part of application No. 10/087,080, filed on Feb. 27, 2002, now abandoned.
(60) Provisional application No. 60/284,555, filed on Apr. 17, 2001.
(60) Provisional application No. 60/281,149, filed on Apr. 2, 2001.
(60) Provisional application No. 60/272,206, filed on Feb. 27, 2001.

(51) Int. Cl. C12Q 1/68; G01N 33/574

(52) U.S. Cl. 435/6; 435/7.23

(57) ABSTRACT

The disclosure provides, among other things, molecular markers for categorizing the neoplastic state of a patient, methods for using the molecular markers in diagnostic tests, nucleic acid and amino acid sequences related to the molecular markers, reagents for detection of molecular markers, and methods for identifying candidate molecular markers in highly parallel gene expression data.

### CROSS-REFERENCE TO RELATED APPLICATIONS

This application is a continuation-in-part of U.S. patent application Ser. No. 10/649,591, filed Aug. 26, 2003, which is a continuation-in-part of U.S. patent application Ser. No. 10/274,177, filed Oct. 18, 2002, which is a continuation-in-part of U.S. patent application Ser. No. 10/229,345, filed Aug. 26, 2002. This application is also a continuation-in-part of U.S. patent application Ser. No. 10/087,080, filed Feb. 27, 2002, which claims the benefit of U.S. Provisional Application No. 60/272,206, filed Feb. 27, 2001, and claims the benefit of U.S. Provisional Application No. 60/281,149, filed Apr. 2, 2001, and claims the benefit of U.S. Provisional Application No. 60/284,555, filed Apr. 17, 2001. All of the aforementioned patent applications are incorporated herein by reference.

### FUNDING

Work described herein was funded, in part, by grant number 1 U01 CA-88130-01 from the National Cancer Institute. The United States government has certain rights in the invention.

### BACKGROUND

Colorectal cancer, also referred to herein as colon cancer, is the second leading cause of cancer mortality in the adult American population. An estimated 135,000 new cases of colon cancer occur each year. Although many people die of colon cancer, early stage colon cancers are often treatable by surgical removal (resection) of the affected tissue. Surgical treatment can be combined with chemotherapeutic agents to achieve an even higher survival rate in certain colon cancers. However, the survival rate drops to 5% or less over five years in patients with metastatic (late stage) colon cancer.

Effective screening and early identification of affected patients coupled with appropriate therapeutic intervention is proven to reduce the number of colon cancer mortalities. It is estimated that 74,000,000 older Americans would benefit from regular screening for colon cancer and precancerous colon adenomas (together, adenomas and colon cancers may be referred to as colon neoplasias). However, present systems for screening for colon neoplasia are inadequate. For example, the Fecal Occult Blood Test involves testing a stool sample from a patient for the presence of blood. This test is relatively simple and inexpensive, but it often fails to detect colon neoplasia (low sensitivity) and often even when blood is detected in the stool, a colon neoplasia is not present (low specificity). Flexible sigmoidoscopy involves the insertion of a short scope into the rectum to visually inspect the lower third of the colon. Because the sigmoidoscope is relatively short, it is also a relatively uncomplicated diagnostic method. However, nearly half of all colon neoplasia occurs in the upper portions of the colon that can not be viewed with the sigmoidoscope. Colonoscopy, in which a scope is threaded through the entire length of the colon, provides a very reliable method of detecting colon neoplasia in a subject, but colonoscopy is costly, time consuming and requires sedation of the patient.

Modern molecular biology has made it possible to identify proteins and nucleic acids that are specifically associated with certain physiological states. These molecular markers have revolutionized diagnostics for a variety of health conditions ranging from pregnancy to viral infections, such as HIV.

Researchers generally identify molecular markers for a health condition by searching for genes and proteins that are expressed at different levels in one health condition versus another (e.g. in pregnant women versus women who are not pregnant). Traditional methods for pursuing this research, such as Northern blots and reverse transcriptase polymerase chain reaction, allow a researcher to study only a handful of potential molecular markers at a time. Microarrays, consisting of an ordered array of hundreds or thousands of probes for detection of hundreds or thousands of gene transcripts, allow researchers to gather data on many potential molecular markers in a single experiment. Researchers now face the challenge of sifting through large quantities of microarray-generated gene expression data to identify genes that may be of genuine use as molecular markers to distinguish different health conditions.

Improved systems for identifying high quality candidate molecular markers in large volumes of gene expression data may help to unlock the power of such tools and increase the likelihood of identifying a molecular marker for important disease states, such as colon neoplasia. Effective molecular markers for colon neoplasia could potentially revolutionize the diagnosis, management and overall health impact of colon cancer.

### BRIEF SUMMARY

This application is based at least in part on the selection of useful molecular markers of colon neoplasia. Colon neoplasia is a multi-stage process involving progression from normal healthy tissues to the development of pre-cancerous colon adenomas to more invasive stages of colon cancer such as the Dukes A and Dukes B stages and finally to metastatic stages such as Dukes C and Dukes D stages of colon cancer.

In one aspect, this application provides molecular markers that are useful in the detection or diagnosis of colon neoplasia. In certain embodiments, molecular markers described in the application are helpful in distinguishing normal subjects from those who are likely to develop colon neoplasia or are likely to harbor a colon adenoma. In other aspects the invention provides molecular markers that may be useful in distinguishing subjects who are either normal or precancerous from those who have colon cancer. In another embodiment, the application provides markers that help in staging the colon cancer in patients. In still other embodiments the application contemplates the use of one or more of the molecular markers described herein for the detection, diagnosis, and staging of colon neoplasias.

In one aspect the application provides a method of screening a subject for a condition associated with increased levels of one or more molecular markers that are indicative of colon neoplasia such as for example ColoUp1-ColoUp8 and osteopontin. In a preferred embodiment, the application provides a method for screening a subject for conditions associated with secreted markers such as ColoUp1 or ColoUp2, by detecting in a biological sample an amount of ColoUp1 or ColoUp2 and comparing the amount of ColoUp1 and ColoUp2 found in the subject to one or more of the following: a predetermined standard, the amount of ColoUp1 or ColoUp2 detected in a normal sample from the subject, the subject's historical baseline level of ColoUp1 or ColoUp2, or the ColoUp1 or ColoUp2 level detected in a different, normal subject (a control subject). Detection of a level of ColoUp1 and ColoUp2 in the subject that is greater than that of the predetermined standard or that is increased from a subject's past baseline is indicative of a condition such as colon neoplasia. In certain aspects, an increase in the amount of ColoUp1 or ColoUp2 as compared to the subject's historical baseline would be indicative of a new neoplasm, or progression of an existing neoplasm. Similarly, a decrease in the amount of ColoUp1 or ColoUp2 as compared to the subject's historical baseline would be indicative of regression on an existing neoplasm

In one aspect the molecular markers described herein are encoded by a nucleic acid sequence that is at least 90%, 95%, 98%, 99%, 99.3%, 99.5% or 99.7% identical to the nucleic acid sequence of SEQ ID Nos: 4-12, and more preferably to the nucleic acid sequences as set forth in SEQ ID Nos: 4-5. In another aspect, the application provides markers that are encoded by a nucleic acid sequence that hybridizes under high stringency conditions to the nucleic acid sequences of SEQ ID Nos: 4-12, more preferably to the nucleic acid sequences as set forth in SEQ ID Nos: 4-5.

In another aspect the application provides molecular markers that are diagnostic of colon neoplasia, said markers having an amino acid sequence that is at least 90%, 95%, 98%, 99%, 99.3%, 99.5% or 99.7% identical to the amino acid sequence as set forth in SEQ ID Nos: 1-3 or 13-20, more preferably the amino acid sequence as set forth in SEQ ID Nos: 3 and 14.

In one aspect, the application provides methods for detecting secreted polypeptide forms of a ColoUp1-ColoUp8 polypeptide or osteopontin in biological samples. In other aspects, the application provides methods for imaging a colon neoplasm by targeting antibodies to any one of the markers ColoUp1 through ColoUp8 described herein, and in preferred embodiments, the antibodies are targeted to ColoUp3. In certain aspects, the application provides methods for administering a imaging agent comprising a targeting moiety and an active moiety. The targeting moiety may be an antibody, Fab, F(Ab)2, a single chain antibody or other binding agent that interacts with an epitope specified by a polypeptide sequence having an amino acid sequence as set forth in SEQ ID Nos: 1-3 and 13-20. The active moiety may be a radioactive agent, such as radioactive technetium, radioactive indium, or radioactive iodine. The imaging agent is administered in an amount effective for diagnostic use in a mammal such as a human and the localization and accumulation of the imaging agent is then detected. The localization and accumulation of the imaging agent may be detected by radioscintigraphy, nuclear magnetic resonance imaging, computed tomography or positron emission tomography.

In a preferred embodiment, the application provides methods for detecting a polypeptide comprising an amino acid sequence as set forth in one of SEQ ID Nos: 1-3. As will be apparent to the skilled artisan, the molecular markers described herein may be detected in a number of ways such as by various assays, including antibody-based assays. Examples of antibody-based assays include immunoprecipitation assays, Western blots, radioimmunoassays or enzyme-linked immunosorbent assays (ELISAs). Molecular markers described herein may be detected by assays that do not employ an antibody, such as by methods employing two-dimensional gel electrophoresis, methods employing mass spectroscopy, methods employing suitable enzymatic activity assays, etc. In a preferred embodiment the application provides methods for the detection of secreted markers such as ColoUp1 or ColoUp2 polypeptides in blood, blood fractions (such as blood serum or blood plasma), urine or stool samples. Increased levels of these markers may be associated with a number of conditions such as for example colon neoplasia, including colon adenomas, colon cancer, and metastatic colon cancer. In certain aspects the application provides methods including the detection of more than one marker that is indicative of colon neoplasia such as methods for detecting both ColoUp1 and ColoUp2. In yet another aspect, combinations of the ColoUp markers may be useful, for instance, a combination of tests including testing biological samples for secreted markers such as ColoUp1 or ColoUp2 in combination with testing for transmembrane markers such as ColoUp3 as targets for imaging agents.

In yet another aspect, the application provides a method of determining whether a subject is likely to develop colon cancer or is more likely to harbor a precancerous colon adenoma by detecting the presence or absence of the molecular markers as set forth in SEQ ID Nos: 1-3. Detection of combinations of these markers is also helpful in staging the colon neoplasias.

In yet another aspect, the application provides markers that are useful in distinguishing normal and precancerous subjects from those subjects having colon cancer. In certain embodiments, the application contemplates determining the levels of markers provided herein such as ColoUp1 through ColoUp8 and osteopontin. In one aspect, markers such as ColoUp6 and osteopontin are helpful in distinguishing between the category of patients that are normal or have precancerous colon adenomas and the category of patients having colon cancer. In another aspect, the application provides detection of one or more of said markers in determining the stages of colon neoplasia.

In certain aspect, the invention provides an immunoassay for determining the presence of any one of the polypeptides having an amino acid sequence as set forth in SEQ ID Nos: 1-3 and 13-20, more preferably any one of the polypeptides having an amino acid sequence as set forth in SEQ ID Nos: 1-3 in a biological sample. The method includes obtaining a biological sample and contacting the sample with an antibody specific for a polypeptide having an amino acid sequence as set forth in SEQ ID Nos: 1-3 and detecting the binding of the antibody.

In some aspects, the application provides methods for the detection of a molecular marker in a biological sample such as blood, including blood fractions such as serum or plasma. For instance, the blood sample obtained from a patient may be further processed such as by fractionation to obtain blood serum, and the serum may then be enriched for certain polypeptides. The serum so enriched is then contacted with an antibody that is reactive with an epitope of the desired marker polypeptide.

In yet another embodiment, the application provides methods for determining the appropriate therapeutic protocol for a subject. For example detection of a colon neoplasia provides the treating physician valuable information in determining whether intensive or invasive protocols such as colonoscopy, surgery or chemotherapy would be needed for effective diagnosis or treatment. Such detection would be helpful not only for patients not previously diagnosed with colon neoplasia but also in those cases where a patient has previously received or is currently receiving therapy for colon cancer, the presence or absence or a change in the level of the molecular markers set forth herein may be indicative that the subject is likely to have a relapse or a progressive, or a persistent colon cancer.

In certain aspects, the application provides molecular markers of colon neoplasia such as ColoUp1 through ColoUp8. In certain instances these markers are secreted proteins such as ColoUp1, ColoUp2 and osteopontin, and are useful for detecting and diagnosing colon neoplasia. In other aspects, these markers may be transmembrane proteins such as ColoUp3 and may be useful as targets for imaging agents, e.g. as targets to label cells of a neoplasm.

In one aspect, the application provides isolated, purified or recombinant polypeptides having an amino acid sequence that is at least 90%, 95% or 98-99% identical to an amino acid sequence as set forth in SEQ ID Nos: 1-3 or an amino acid sequence as set forth in SEQ ID Nos: 13-20. In a more preferred embodiment, the application provides an amino acid sequence that is at least 90%, 95%, 98-99%, 99.3%, 99.5% or 99.7% identical to the amino acid sequence as set forth in SEQ ID No: 3 or SEQ ID No: 14. The application also provides fusion proteins comprising the ColoUp proteins described herein fused to a heterologous protein. In certain embodiments, such polypeptides are useful, for example, for generating antibodies or for use in screening assays to identify candidate therapeutics.

In other aspects the application provides for nucleic acid sequences encoding the polypeptides as set forth in SEQ ID Nos: 1-3 and 13-20. In one aspect the application provides nucleic acids comprising nucleic acid sequences that are at least 90%, 95%, 98-99%, 99.3%, 99.5% or 99.7% identical to the nucleic acid sequence in SEQ ID Nos: 4-12, more preferably 4-5. Also contemplated herein are vectors comprising the nucleic acid sequences set forth in SEQ ID Nos: 4-12, more preferably SEQ ID Nos: 4-5, and host cells expressing the nucleic acid sequences.

In another aspect, the application provides an antibody that interacts with an epitope specified by one of SEQ ID Nos: 1-3 and 13-20 or portions thereof, more preferably SEQ ID Nos: 1-3 or portions thereof. In a preferred embodiment the antibody is useful for detecting colon adenomas and interacts with an epitope specified by one of SEQ ID Nos: 1-3. In certain aspects the application provides for generating such antibodies, including methods for generating monoclonal and polyclonal antibodies, as well as methods for generating other types of antibodies. In other aspects, the application also provides a hybridoma cell line capable of producing an antibody that interacts with an epitope specified by SEQ ID Nos: 1-3 and 13-20, more preferably SEQ ID Nos: 1-3, or portions thereof In yet other embodiments, the antibody may be a single chain antibody.

In yet other embodiments, the application provides a kit for detecting colon neoplasia in a biological sample, Such kits include one or more antibodies that are capable of interacting with an epitope specified by one of SEQ ID Nos: 1-3 and 13-20, more preferably with an epitope specified by one of SEQ ID Nos: 1-3. In more preferred embodiments, the antibodies may be detectably labeled, such as for example with an enzyme, a fluorescent substance, a chemiluminescent substance, a chromophore, a radioactive isotope or a complexing agent.

In certain embodiments, the application provides the identity of ColoUp1 and ColoUp2 polypeptides that are secreted into the serum in vivo, and that are secreted across the apical and basolateral cell surfaces in cultured intestinal cells. Accordingly, in certain embodiments, the application provides methods for detecting whether a subject to likely to have a colon neoplasia comprising: a) obtaining a biological sample from said subject; and b) detecting one or more polypeptides selected from among: one or more secreted ColoUp1 polypeptides and one or more secreted ColoUp2 polypeptides, wherein the presence of said one or more polypeptides is indicative of colon neoplasia.

In certain embodiments, a secreted ColoUp2 polypeptide is selected from among: a) a secreted polypeptide produced by the expression of a nucleic acid that is at least 95% identical to the amino acid sequence of SEQ ID No: 5; b) a secreted polypeptide produced by the expression of a nucleic acid that is a naturally occurring variant of SEQ ID No: 5; c) a secreted polypeptide produced by the expression of a nucleic acid that hybridizes under stringent conditions to a nucleic acid sequence of SEQ ID No: 5; d) a secreted polypeptide having a sequence that is at least 95% identical to the amino acid sequence of SEQ ID No: 3; and e) a secreted polypeptide having a sequence that is at least 95% identical to the amino acid sequence of SEQ ID No: 21. Optionally, the secreted ColoUp2 polypeptide is produced by the expression of a nucleic acid having the sequence of SEQ ID No: 5, and preferably the secreted ColoUp2 polypeptide is produced by the expression of a nucleic acid sequence that is at least 98%, 99% or 100% identical to the nucleic acid sequence of SEQ ID No: 5. In certain embodiments, the secreted ColoUp2 polypeptide has an amino acid sequence that is at least 98%, 99% or 100% identical to an amino acid sequence selected from among SEQ ID No: 3 and SEQ ID No:21. In certain embodiments, the secreted ColoUp1 polypeptide is selected from among: a) a secreted polypeptide produced by the expression of a nucleic acid that is at least 95% identical to the amino acid sequence of SEQ ID No: 4; b) a secreted polypeptide produced by the expression of a nucleic acid that is a naturally occurring variant of SEQ ID No: 4; c) a secreted polypeptide produced by the expression of a nucleic acid that hybridizes under stringent conditions to a nucleic acid sequence of SEQ ID No: 4; d) a secreted polypeptide having a sequence that is at least 95% identical to the amino acid sequence of SEQ ID No: 1; and e) a secreted polypeptide having a sequence that is at least 95% identical to the amino acid sequence of SEQ ID No: 2. Optionally, the secreted ColoUp1 polypeptide is produced by the expression of a nucleic acid having a sequence that is at least 95%, 98, 99% or 100% identical to the nucleic acid sequence of SEQ ID No: 4. Preferably, the secreted ColoUp1 polypeptide has an amino acid sequence that is at least 95%, 98%, 99% or 100% identical to an amino acid sequence selected from among SEQ ID No: 1 and SEQ ID No:2. Optionally, for detection of basolaterally secreted ColoUp1 or ColoUp2 polypeptides, the biological sample is a blood sample or a fraction derived from blood, such as serum, plasma, cells, or a fraction enriched for apically secreted ColoUp1 or ColoUp2 polypeptide. Optionally, for detection of basolaterally secreted ColoUp1 or ColoUp2 polypeptides, the biological sample is a urine sample or a fraction derived from urine. Optionally, for detection of apically secreted ColoUp1 or ColoUp2 polypeptides, the biological sample is derived from the inner wall and/or lumen of the intestinal tract, such as intestinal mucous or other fluid, excreted stool and stool removed from within the colon. In certain embodiments, the polypeptide is detected by an assay that employs an antibody, such as an immunoprecipitation assay, a Western blot, a radioimmunoassays or an enzyme-linked immunosorbent assay (ELISA). Optionally, an assay comprises contacting the biological sample with an antibody that interacts with a secreted ColoUp1 polypeptide or a secreted ColoUp2 polypeptide. An antibody may, for example, interact with an epitope of an amino acid sequence selected from among: SEQ ID No: 1 and SEQ ID No: 2. An antibody may, for example, interact with an epitope of an amino acid sequence selected from among: SEQ ID No: 3 and SEQ ID No: 21. Optionally, the antibody is detectably labeled, such as with an enzyme, a fluorescent substance, a chemiluminescent substance, a chromophore, a radioactive isotope or a complexing agent. Optionally, the amount of at least one secreted ColoUp1 polypeptide and/or at least one secreted ColoUp2 polypeptide in the biological sample is compared to a predetermined standard (e.g., a known amount of purified ColoUp1 or ColoUp2 polypeptide). Optionally, the amount of at least one secreted ColoUp 1 polypeptide and/or at least one secreted ColoUp2 polypeptide in the biological sample is compared to the subject's historical baseline. In certain embodiments, the presence of at least one secreted ColoUp1 polypeptide and/or at least one secreted ColoUp2 polypeptide is indicative that the subject is likely to harbor a colon adenoma or a colon cancer. In certain embodiments, the presence of at least one secreted ColoUp1 polypeptide and/or at least one secreted ColoUp2 polypeptide may be used in determining the therapeutic protocol to be administered to a subject having a colon neoplasia, and the subject may not have been previously diagnosed with colon cancer or the subject may have previously received or is currently receiving a therapy for colon cancer, wherein the presence of at least one secreted ColoUp1 polypeptide and/or at least one secreted ColoUp2 polypeptide indicates that the subject is likely to have a relapse or a persistent or progressive colon cancer. The detection of said secreted polypeptide may indicate the presence of a variety of neoplasias in a subject, such as a colon adenoma, a colon cancer and a metastatic colon cancer. Optionally, a method involves detecting both at least one secreted ColoUp1 polypeptide and at least one secreted ColoUp2 polypeptide in the biological sample.

In certain embodiments, the application provides kits for detecting one or more molecular markers of colon neoplasia in a biological sample. A kit may comprise a) an antibody which interacts with an epitope of a secreted ColoUp1 polypeptide or a secreted ColoUp2 polypeptide; and b) instructions for use. Optionally, the antibody interacts with an epitope of a polypeptide selected from among: the polypeptide of SEQ ID No:1, the polypeptide of SEQ ID No:2, the polypeptide of SEQ ID No:3 and the polypeptide of SEQ ID No:21. Optionally, the antibody is detectably labeled.

In certain embodiments, the application provides a novel purified polypeptide, which is a portion of ColoUp2 that is found in serum. Such a polypeptide may consist essentially of an amino acid sequence that is at least 95%, 98%, 99% or 100% identical to the sequence of SEQ ID No: 21. By “consisting essentially” is meant that there may be, in addition to the indicated amino acid sequence, a variety of modifications, such as phosphorylations, glycosylations, disulfide bonds, unusual or modified amino acids, etc.

In certain embodiments, the application provides novel fusion proteins comprising a first polypeptide domain and a second polypeptide domain, wherein the first polypeptide domain consists essentially of an amino acid sequence that is at least 95%, 98%, 99% or 100% identical to an amino acid sequence of SEQ ID No. 21. The second polypeptide domain may be a domain selected from the group consisting of: a detection domain, a purification domain and an antigenic domain.

In certain embodiments, the application provides antibodies that bind specifically to a ColoUp2 polypeptide consisting essentially of the amino acid sequence of SEQ ID No: 21. The antibody may binds the ColoUp2 polypeptide with a dissociation constant of less than 10⁻⁶M, 10⁻⁷M, 10⁻⁸M or 10⁻⁹M. The antibody may be essentially any type of antibody, including polyclonal, monoclonal, and single chain antibodies, or other fragments. For diagnostic use, there may be little benefit to having a humanized antibody, however, humanized antibodies are highly desirable for therapeutic uses. Preferably, a diagnostic antibody is effective for detecting the ColoUp2 polypeptide in a biological sample, such as a blood, stool or urine sample, or a fraction thereof. Optionally, the antibody is effective for detecting the ColoUp2 polypeptide in a sample comprising cells from a colon neoplasia. The application further provides methods for making such antibodies in a variety of ways. For example, a monoclonal antibody may be produced in a method comprising: (a) administering to a mouse an amount of an immunogenic composition comprising the ColoUp2 polypeptide effective to stimulate a detectable immune response; (b) obtaining antibody-producing cells from the mouse and fusing the antibody-producing cells with myeloma cells to obtain antibody-producing hybridomas; (c) testing the antibody-producing hybridomas to identify a preferred hybridoma, wherein the preferred hybridoma is a hybridoma that produces a monocolonal antibody that binds specifically to the ColoUp2 polypeptide; (d) culturing the preferred hybridoma cell culture that produces the monoclonal antibody that binds specifically to the ColoUp2 polypeptide; and (e) obtaining the monoclonal antibody that binds specifically to the ColoUp2 polypeptide from the cell culture. Optionally, the antibody-producing hybridomas comprises testing whether the antibody-producing hybridomas produce an antibody that binds to the ColoUp2 polypeptide in an assay selected from the group consisting of: an enzyme-linked immunosorbent assay, a Bia-core assay and an immunoprecipitation assay.

In further aspects, the disclosure provides nucleotide sequences of genes that are up- and down-regulated in colorectal cancer cells, including metastatic colorectal cancer cells. Such genes and the proteins they encode are useful for diagnostic and prognostic purposes, and also as targets for screening for therapeutic compounds that modulate colorectal cancer, such as antibodies. The methods of detecting nucleic acids of the invention or their encoded proteins can be used for a number of purposes. Examples include, early detection of colon cancers, monitoring and early detection of relapse following treatment of colon cancers, monitoring response to therapy of colon cancers, determining prognosis of colon cancers, directing therapy of colon cancers, selecting patients for postoperative chemotherapy or radiation therapy, selecting therapy, determining tumor prognosis, treatment, or response to treatment, and early detection of precancerous colon adenomas.

In a further aspect, the application provides a method of detecting a metastatic colorectal cancer-associated transcript in a cell from a patient, the method comprising contacting a biological sample from the patient with a polynucleotide that selectively hybridizes to a sequence at least 80% identical to a ColoUp1 sequence, such as that of SEQ ID No. 4. In one embodiment, the polynucleotide selectively hybridizes to a sequence at least 95% identical to a sequence as shown in SEQ ID No.4. In another embodiment, the polynucleotide comprises a sequence as shown in SEQ ID No.4, or the portion thereof that encodes a ColoUp1 polypeptide. In one embodiment, the biological sample is a tissue sample. In another embodiment, the biological sample comprises isolated nucleic acids, e.g., mRNA. In one embodiment, the polynucleotide is labeled, e.g., with a fluorescent label. In one embodiment, the polynucleotide is immobilized on a solid surface. In one embodiment, the patient is undergoing a therapeutic regimen to treat metastatic colorectal cancer. In another embodiment, the patient is suspected of having metastatic colorectal cancer. In one embodiment, the patient is a human. In one embodiment, the method further comprises the step of amplifying nucleic acids before the step of contacting the biological sample with the polynucleotide. In another aspect, the application provides methods of detecting polypeptide encoded by a metastatic colorectal cancer-associated transcript in a cell from a patient, the method comprising contacting a biological sample from the patient with an antibody that specifically binds a polypeptide encoded by a sequence at least 80% identical to a sequence as shown in SEQ ID No. 4.

In another aspect, the present invention provides a method of monitoring the efficacy of a therapeutic treatment of metastatic colorectal cancer, the method comprising the steps of: (i) providing a biological sample from a patient undergoing the therapeutic treatment; and (ii) determining the level of a metastatic colorectal cancer-associated transcript in the biological sample by contacting the biological sample with a polynucleotide that selectively hybridizes to a sequence at least 80% identical to a sequence as shown in SEQ ID No.4, thereby monitoring the efficacy of the therapy. In one embodiment, the method further comprises the step of: (iii) comparing the level of the metastatic colorectal cancer-associated transcript to a level of the metastatic colorectal cancer-associated transcript in a biological sample from the patient prior to, or earlier in, the therapeutic treatment.

In another aspect, the present invention provides a method of monitoring the efficacy of a therapeutic treatment of metastatic colorectal cancer, the method comprising the steps of: (i) providing a biological sample from a patient undergoing the therapeutic treatment; and (ii) determining the level of a metastatic colorectal cancer-associated antibody in the biological sample by contacting the biological sample with a polypeptide encoded by a polynucleotide that selectively hybridizes to a sequence at least 80% identical to a sequence as shown SEQ ID No.4, wherein the polypeptide specifically binds to the metastatic colorectal cancer-associated antibody, thereby monitoring the efficacy of the therapy. In one embodiment, the method further comprises the step of: (iii) comparing the level of the metastatic colorectal cancer-associated antibody to a level of the metastatic colorectal cancer-associated antibody in a biological sample from the patient prior to, or earlier in, the therapeutic treatment.

In another aspect, the present invention provides a method of monitoring the efficacy of a therapeutic treatment of metastatic colorectal cancer, the method comprising the steps of: (i) providing a biological sample from a patient undergoing the therapeutic treatment; and (ii) determining the level of a metastatic colorectal cancer-associated polypeptide in the biological sample by contacting the biological sample with an antibody, wherein the antibody specifically binds to a polypeptide encoded by a polynucleotide that selectively hybridizes to a sequence at least 80% identical to a sequence as shown in SEQ ID No. 4, thereby monitoring the efficacy of the therapy. In one embodiment, the method further comprises the step of: (iii) comparing the level of the metastatic colorectal cancer-associated polypeptide to a level of the metastatic colorectal cancer-associated polypeptide in a biological sample from the patient prior to, or earlier in, the therapeutic treatment.

In one aspect, the application provides an isolated nucleic acid molecule consisting of a polynucleotide sequence as shown in SEQ ID No. 4, or the portion thereof that encodes a polypeptide of any of SEQ ID Nos. 1, 2 and 13. In one embodiment, an expression vector or cell comprises the isolated nucleic acid.

In one aspect, the application provides an isolated polypeptide which is encoded by a nucleic acid molecule having polynucleotide sequence as shown in SEQ ID No.4.

In another aspect, the present invention provides an antibody that specifically binds to an isolated polypeptide which is encoded by a nucleic acid molecule having polynucleotide sequence as shown in SEQ ID No. 4. In one embodiment, the antibody is conjugated to an effector component, e.g., a fluorescent label, a radioisotope or a cytotoxic chemical. In one embodiment, the antibody is an antibody fragment. In another embodiment, the antibody is humanized. In one aspect, the present invention provides a method of detecting a metastatic colorectal cancer cell in a biological sample from a patient, the method comprising contacting the biological sample with an antibody as described herein. In another aspect, the present invention provides a method of detecting antibodies specific to metastatic colorectal cancer in a patient, the method comprising contacting a biological sample from the patient with a polypeptide encoded by a nucleic acid comprises a sequence from SEQ ID No. 4.

In another aspect, the present invention provides a method for identifying a compound that modulates a metastatic colorectal cancer-associated polypeptide, the method comprising the steps of: (i) contacting the compound with a metastatic colorectal cancer-associated polypeptide, the polypeptide encoded by a polynucleotide that selectively hybridizes to a sequence at least 80% identical to a sequence as shown in SEQ ID No. 4; and (ii) determining the functional effect of the compound upon the polypeptide. In one embodiment, the functional effect is a physical effect, an enzymatic effect, or a chemical effect. In one embodiment, the polypeptide is expressed in a eukaryotic host cell or cell membrane. In another embodiment, the polypeptide is recombinant. In one embodiment, the functional effect is determined by measuring ligand binding to the polypeptide.

In another aspect, the present invention provides a method of inhibiting proliferation of a metastatic colorectal cancer-associated cell to treat colorectal cancer in a patient, the method comprising the step of administering to the subject a therapeutically effective amount of a compound identified as described herein. In one embodiment, the compound is an antibody.

In another aspect, the present invention provides a drug screening assay comprising the steps of: (i) administering a test compound to a mammal having colorectal cancer or a cell isolated therefrom; (ii) comparing the level of gene expression of a polynucleotide that selectively hybridizes to a sequence at least 80% identical to a sequence as shown in SEQ ID No. 4 in a treated cell or mammal with the level of gene expression of the polynucleotide in a control cell or mammal, wherein a test compound that modulates the level of expression of the polynucleotide is a candidate for the treatment of colorectal cancer. In one embodiment, the control is a mammal with colorectal cancer or a cell therefrom that has not been treated with the test compound. In another embodiment, the control is a normal cell or mammal.

In another aspect, the present invention provides a method for treating a mammal having colorectal cancer comprising administering a compound identified by the assay described herein.

In another aspect, the present invention provides a pharmaceutical composition for treating a mammal having colorectal cancer, the composition comprising a compound identified by the assay described herein and a physiologically acceptable excipient.

The embodiments and practices of the present invention, other embodiments, and their features and characteristics, will be apparent from the description, figures and claims that follow, with all of the claims hereby being incorporated by this reference into this Summary.

### BRIEF DESCRIPTION OF THE DRAWINGS

FIG. 1 shows the amino acid sequences (SEQ ID NOs: 1 and 2) of secreted ColoUp1protein. A. An N-terminal signal peptide is cleaved between amino acids 30-31 of the full-length ColoUp1 protein; B. An N-terminal signal peptide is cleaved between amino acids 33-34 of the full-length ColoUp1 protein.

FIG. 2 shows the amino acid sequence (SEQ ID NO: 3) of secreted ColoUp2 protein.

FIG. 3 shows the nucleic acid sequence (SEQ ID NO: 4) of ColoUp1.

FIG. 4 shows the nucleic acid sequence (SEQ ID NO: 5) of ColoUp2.

FIG. 5 shows the nucleic acid sequence (SEQ ID NO: 6) of Osteopontin.

FIG. 6 shows the nucleic acid sequence (SEQ ID NO: 7) of ColoUp3.

FIG. 7 shows the nucleic acid sequence (SEQ ID NO: 8) of ColoUp4.

FIG. 8 shows the nucleic acid sequence (SEQ ID NO: 9) of ColoUp5.

FIG. 9 shows the nucleic acid sequence (SEQ ID NO: 10) of ColoUp6.

FIG. 10 shows the nucleic acid sequence (SEQ ID NO: 11) of ColoUp7.

FIG. 11 shows the nucleic acid sequence (SEQ ID NO: 12) of ColoUp8.

FIG. 12 shows the amino acid sequence (SEQ ID NO: 13) of full-length ColoUp1 protein.

FIG. 13 shows the amino acid sequence (SEQ ID NO: 14) of full-length ColoUp2 protein.

FIG. 14 shows the amino acid sequence (SEQ ID NO: 15) of full-length Osteopontin protein.

FIG. 15 shows the amino acid sequence (SEQ ID NO: 16) of full-length ColoUp3 protein.

FIG. 16 shows the amino acid sequence (SEQ ID NO: 17) of full-length ColoUp4 protein.

FIG. 17 shows the amino acid sequence (SEQ ID NO: 18) of full-length ColoUp5 protein.

FIG. 18 shows the amino acid sequence (SEQ ID NO: 19) of full-length ColoUp6 protein.

FIG. 19 shows the amino acid sequence (SEQ ID NO: 20) of full-length ColoUp8 protein.

FIG. 20 is a graphical display of ColoUp1 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 21 is a graphical display of ColoUp2 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 22 is a graphical display of Osteopontin expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 23 is a graphical display of ColoUp3 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 24 is a graphical display of ColoUp4 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 25 is a graphical display of ColoUp5 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 26 is a graphical display of ColoUp6 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 27 is a graphical display of ColoUp7 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 28 is a graphical display of ColoUp8 expression levels measured by micro-array profiling in different samples. A. In normal colon epithelial strips, normal liver, and colonic muscle; B. In premalignant colon adenomas as well as in colon cancers of Dukes stages B, Dukes stage C, and Duke stages D; C. In colon cancer liver metastasis; D. In colon cancer cell lines, colon cancer xenografts grown in athymic mice, MSI cell lines, and V330 cell lines treated with TGFβ.

FIG. 29 shows northern blot analysis of ColoUp1 mRNA levels in normal colon tissues and colon cancer cell lines or tissues. A. In normal colon tissue samples and a group of colon cancer cell lines; B. and C. In normal colon tissues and colon neoplasms from 15 individuals with colon cancers and one individual with a colon adenoma.

FIG. 30 shows detection of T7 epitope-tagged ColoUp1 protein levels in transfected FET cells and Vaco400 cells. A. Secretion of epitope-tagged ColoUp1 protein in V400 cell growth media by Western blot (“T” are transfectants with an epitope tagged ColoUp1 expression vector; “C” are transfectants with an empty control vector); B. Expression of T7 epitope-tagged ColoUp1 protein in transfected FET cells and V400 cells by Western blot (left panel), and secretion of epitope-tagged ColoUp1 protein in growth media by serial immunoprecipitation and Western blot (right panel)(Cell extract amounts loaded: FET=75 mg/well; V400=31.1 mg/well; Volume of media used for immuno-precipitation=1 ml of 20 ml).

FIG. 31 shows northern blot analysis of ColoUp2 mRNA levels in normal colon tissue samples and a group of colon cancer cell lines (top panel). The bottom panel shows the ethidium bromide stained gel corresponding to the blot.

FIG. 32 shows detection of V5 epitope-tagged ColoUp2 protein levels in transfected SW480 cells and Vaco400 cells (24 hours and 48 hours after trnasfection). Expression of epitope-tagged ColoUp2 protein in transfected cells by Western blot (right panel), and secretion of epitope-tagged ColoUp2 protein in growth media by serial immunoprecipitation and Western blot (left panel).

FIG. 33 shows two northern blot analysis of ColoUp5 mRNA levels in normal colon tissues and a group of colon cancer cell lines (top panels). The bottom panels show the ethidium bromide stained gel corresponding to the blot.

FIG. 34 illustrates an alignment of the human, mouse, and rat ColoUp5 (FoxQ1) amino acid sequences.

FIG. 35 illustrates an alignment of the human, mouse, and rat ColoUp5 (FoxQ1) nucleic acid sequences.

FIG. 36 shows a western blot of V5 tagged ColoUp2 protein detected by anti-V5 antibody. Lane 1: media supernate from SW480 colon cancer cells transfected with an empty expression vector. Lane 2: media supernate from ColoUp2-V5 expressing cells. Lane 3: size markers. Lane 4 shows assay of serum from a mouse xenografted with control SW480 cells corresponding to lane 1. Lanes 5 and 6 show detection of circulating ColoUp2 proteins in blood from two mice bearing human colon cancer xenografts from ColoUp2-V5 expressing SW480 colon cells shown in lane 2. ColoUp2 is secreted as an 85 KD and a companion 55 KD size protein.

FIG. 37 shows a western blot with anti-V5 antibody of V5 tagged ColoUp1 protein. Lane 1: media supernate from SW480 colon cancer cells transfected with an empty expression vector. Lane 2: media supernate from ColoUp1-V5 expressing SW480 cells. Lane 3 shows assay of serum from a mouse xenografted with control SW480 cells corresponding to lane 1. Lanes 4 shows detection of circulating ColoUp1 proteins in blood from a mouse bearing tumor xenografts from ColoUp1-V5 expressing SW480 cells shown in lane 2. Lane 5: size markers.

FIG. 38 shows, in the upper panel, the purification of ColoUp2 protein. Shown is a Coomassie blue staining of 250 ng (lane 2a) and 500 ng (lane 3a) of a purified ColoUp2 protein preparation. Size markers are in lane 1a. In the lower panel is shown a Coomassie blue stained gel showing purification of His-tagged ColoUp1 protein on Ni-NTA beads. Lane 1 :markers, Lane 2 media from mock transfected cells, Lane 3 purification of media from ColoUp1 transfected cells. Clearly shown is purification to homogeneity of the 180 kd ColoUp protein.

FIG. 39 shows, in the top panel, detection on an anti-V5 western of V5-tagged ColoUp2 protein. Lane 1: media from mock transfected Caco2 cells. Lane 2: detection of secreted ColoUp2 protein from transiently transfected Caco2 cells grown in standard culture dishes. Seen are the typical 85 KD and 55 KD secreted bands (the lane is heavily overloaded and minor degradation products are also visualized). Lane 3: molecular weight markers. Lanes 4-7: detection of ColoUp2 secreted into the basolateral compartment (lower chamber) of transiently transfected Caco2 grown as a monolayer on a transwell filter. Lanes 9-12 show the general absence of ColoUp2 in the corresponding apical apical compartment, with the exception of the 48 hour time point. The table shows the electrical resistance and transfection efficiency (gfp expression) measured at each time point. A dip in the electrical resistance at 48 hours suggests some leakiness of the monolayer at that time point.

FIG. 40: Top panel shows detection on anti-V5 western of V5-tagged ColoUp1 protein. Control lane shows detection of purified recombinant ColoUp1. Identical bands are seen in media harvested on days 1-4 (lanes D1-D4) from both apical and basolateral compartments. The table shows the electrical resistance and transfection efficiency (gfp expression) measured at each time point.

FIG. 41 shows the amino acid sequence of the approximately 55 kDa C-terminal fragment of ColoUp2 that is a prominent secreted and serum form of ColoUp2.

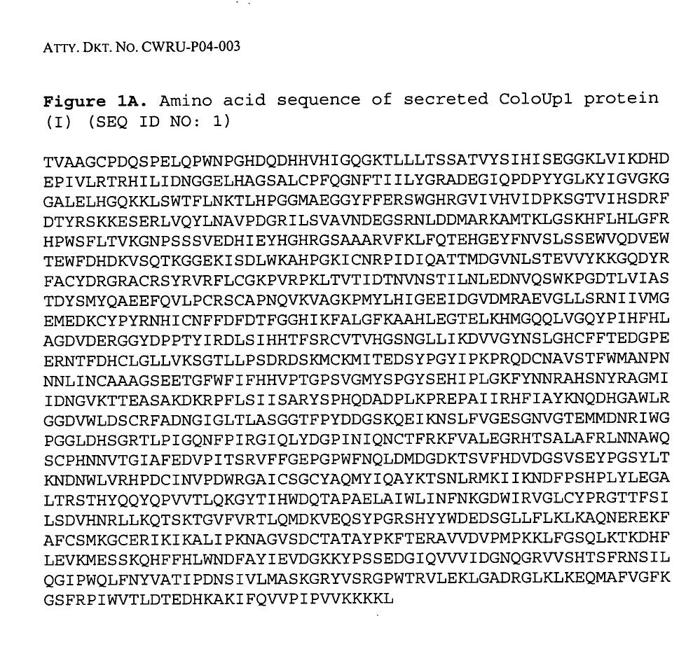

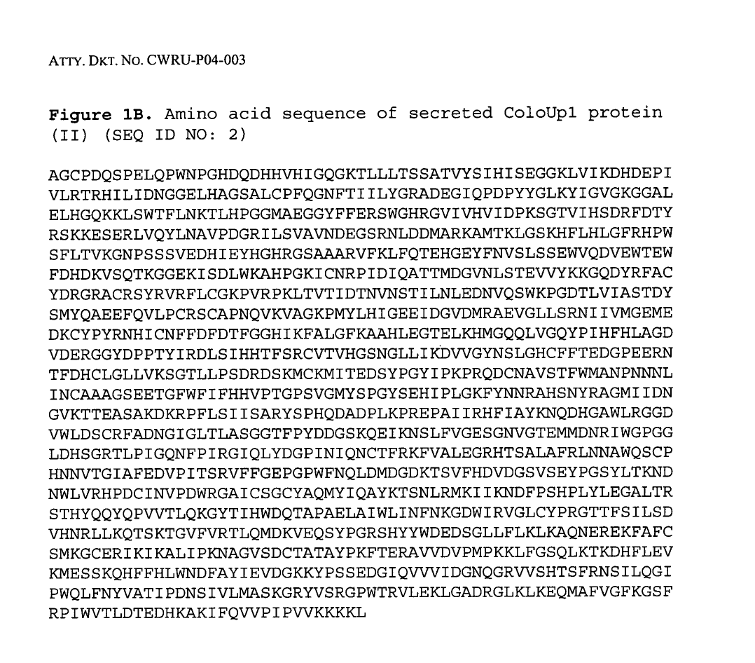

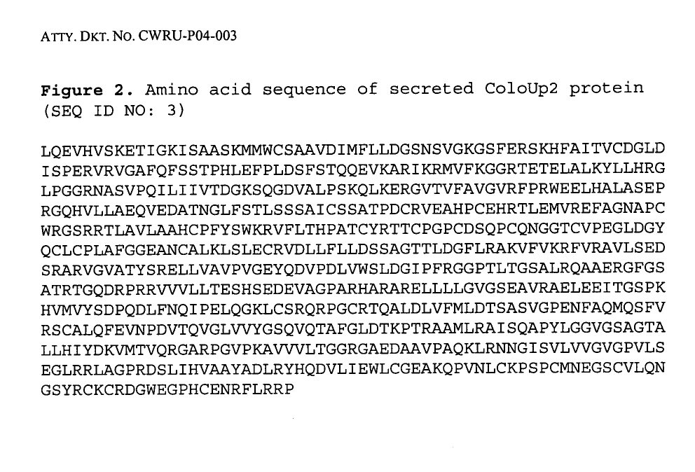

### DETAILED DESCRIPTION

1. Definitions:

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The terms “adenoma”, “colon adenoma” and “polyp” are used herein to describe any precancerous neoplasia of the colon.

The term “antibody” as used herein is intended to include whole antibodies, e.g., of any isotype (IgG, IgA, IgM, IgE, etc), and includes fragments thereof which are also specifically reactive with a vertebrate, e.g., mammalian, protein. Antibodies can be fragmented using conventional techniques and the fragments screened for utility and/or interaction with a specific epitope of interest. Thus, the term includes segments of proteolytically-cleaved or recombinantly-prepared portions of an antibody molecule that are capable of selectively reacting with a certain protein. Non-limiting examples of such proteolytic and/or recombinant fragments include Fab, F(ab′)2, Fab′, Fv, and single chain antibodies (scFv) containing a V[L] and/or V[H] domain joined by a peptide linker. The scFv's may be covalently or non-covalently linked to form antibodies having two or more binding sites. The term antibody also includes polyclonal, monoclonal, or other purified preparations of antibodies and recombinant antibodies.

The term “colon” as used herein is intended to encompass the right colon (including the cecum), the transverse colon, the left colon and the rectum.

The terms “colorectal cancer” and “colon cancer” are used interchangeably herein to refer to any cancerous neoplasia of the colon (including the rectum, as defined above).

The term “ColoUpX” (e.g. ColoUp1, ColoUp2 . . . ColoUp8) is used to refer to a nucleic acid encoding a ColoUp protein or a ColoUp protein itself, as well as distinguishable fragments of such nucleic acids and proteins, longer nucleic acids and polypeptides that comprise distinguishable fragments or full length nucleic acids or polypeptides, and variants thereof. Variants include polypeptides that are at least 90% identical to the relevant human ColoUp SEQ ID Nos. referred to in the application, and nucleic acids encoding such variant polypeptides. In addition, variants include different post-translational modifications, such as glycosylations, methylations, etc. Particularly preferred variants include any naturally occurring variants, such as allelic differences, mutations that occur in a neoplasia and secreted or processed forms. The terms “variants” and “fragments” are overlapping.

As used herein, the phrase “gene expression” or “protein expression” includes any information pertaining to the amount of gene transcript or protein present in a sample, as well as information about the rate at which genes or proteins are produced or are accumulating or being degraded (eg. reporter gene data, data from nuclear runoff experiments, pulse-chase data etc.). Certain kinds of data might be viewed as relating to both gene and protein expression. For example, protein levels in a cell are reflective of the level of protein as well as the level of transcription, and such data is intended to be included by the phrase “gene or protein expression information”. Such information may be given in the form of amounts per cell, amounts relative to a control gene or protein, in unitless measures, etc.; the term “information” is not to be limited to any particular means of representation and is intended to mean any representation that provides relevant information. The term “expression levels” refers to a quantity reflected in or derivable from the gene or protein expression data, whether the data is directed to gene transcript accumulation or protein accumulation or protein synthesis rates, etc.

The term “detection” is used herein to refer to any process of observing a marker, in a biological sample, whether or not the marker is actually detected. In other words, the act of probing a sample for a marker is a “detection” even if the marker is determined to be not present or below the level of sensitivity. Detection may be a quantitative, semi-quantitative or non-quantitative observation.

The terms “healthy”, “normal” and “non-neoplastic” are used interchangeably herein to refer to a subject or particular cell or tissue that is devoid (at least to the limit of detection) of a disease condition, such as a neoplasia, that is associated with increased expression of a ColoUp gene. These terms are often used herein in reference to tissues and cells of the colon. Thus, for the purposes of this application, a patient with severe heart disease but lacking a ColoUp-associated disease would be termed “healthy”.

The term “including” is used herein to mean, and is used interchangeably with, the phrase “including but not limited to”.

As used herein, the term “nucleic acid” refers to polynucleotides such as deoxyribonucleic acid (DNA), and, where appropriate, ribonucleic acid (RNA). The term should also be understood to include analogs of either RNA or DNA made from nucleotide analogs, and, as applicable to the embodiment being described, single-stranded (such as sense or antisense) and double-stranded polynucleotides.

The term “or” is used herein to mean, and is used interchangeably with, the term “and/or”, unless context clearly indicates otherwise.

The term “percent identical” refers to sequence identity between two amino acid sequences or between two nucleotide sequences. Identity can each be determined by comparing a position in each sequence which may be aligned for purposes of comparison. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology/similarity or identity refers to a function of the number of identical or similar amino acids at positions shared by the compared sequences. Various alignment algorithms and/or programs may be used, including FASTA, BLAST or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

The terms “polypeptide” and “protein” are used interchangeably herein.

The term “purified protein” refers to a preparation of a protein or proteins which are preferably isolated from, or otherwise substantially free of, other proteins normally associated with the protein(s) in a cell or cell lysate. The term “substantially free of other cellular proteins” (also referred to herein as “substantially free of other contaminating proteins”) is defined as encompassing individual preparations of each of the component proteins comprising less than 20% (by dry weight) contaminating protein, and preferably comprises less than 5% contaminating protein. Functional forms of each of the component proteins can be prepared as purified preparations by using a cloned gene as described in the attached examples. By “purified”, it is meant, when referring to component protein preparations used to generate a reconstituted protein mixture, that the indicated molecule is present in the substantial absence of other biological macromolecules, such as other proteins (particularly other proteins which may substantially mask, diminish, confuse or alter the characteristics of the component proteins either as purified preparations or in their function in the subject reconstituted mixture). The term “purified” as used herein preferably means at least 80% by dry weight, more preferably in the range of 85% by weight, more preferably 95-99% by weight, and most preferably at least 99.8% by weight, of biological macromolecules of the same type present (but water, buffers, and other small molecules, especially molecules having a molecular weight of less than 5000, can be present). The term “pure” as used herein preferably has the same numerical limits as “purified” immediately above.

A “recombinant nucleic acid” is any nucleic acid that has been placed adjacent to another nucleic acid by recombinant DNA techniques. A “recombinant nucleic acid” also includes any nucleic acid that has been placed next to a second nucleic acid by a laboratory genetic technique such as, for example, transformation and integration, transposon hopping or viral insertion. In general, a recombined nucleic acid is not naturally located adjacent to the second nucleic acid.

The term “recombinant protein” refers to a protein that is produced by expression from a recombinant nucleic acid.

A “sample” includes any material that is obtained or prepared for detection of a molecular marker, or any material that is contacted with a detection reagent or detection device for the purpose of detecting a molecular marker.

A “subject” is any organism of interest, generally a mammalian subject, such as a mouse, and preferably a human subject.

2. Overview

In certain aspects, the invention relates to methods for determining whether a subject is likely or unlikely to have a colon neoplasia. In other aspects, the invention relates to methods for determining whether a patient is likely or unlikely to have a colon cancer. In further aspects, the invention relates to methods for monitoring colon neoplasia in a subject. In further aspects, the invention relates to methods for staging a subject's colon neoplasia. A colon neoplasia is any cancerous or precancerous growth located in, or derived from, the colon. The colon is a portion of the intestinal tract that is roughly three feet in length, stretching from the end of the small intestine to the rectum. Viewed in cross section, the colon consists of four distinguishable layers arranged in concentric rings surrounding an interior space, termed the lumen, through which digested materials pass. In order, moving outward from the lumen, the layers are termed the mucosa, the submucosa, the muscularis propria and the subserosa. The mucosa includes the epithelial layer (cells adjacent to the lumen), the basement membrane, the lamina propria and the musculanris mucosae. In general, the “wall” of the colon is intended to refer to the submucosa and the layers outside of the submucosa. The “lining” is the mucosa.

Precancerous colon neoplasias are referred to as adenomas or adenomatous polyps. Adenomas are typically small mushroom-like or wart-like growths on the lining of the colon and do not invade into the wall of the colon. Adenomas may be visualized through a device such as a colonoscope or flexible sigmoidoscope. Several studies have shown that patients who undergo screening for and removal of adenomas have a decreased rate of mortality from colon cancer. For this and other reasons, it is generally accepted that adenomas are an obligate precursor for the vast majority of colon cancers.

When a colon neoplasia invades into the basement membrane of the colon, it is considered a colon cancer, as the term “colon cancer” is used herein. In describing colon cancers, this specification will generally follow the so-called “Dukes” colon cancer staging system. Other staging systems have been devised, and the particular system selected is, for the purposes of this disclosure, unimportant. The characteristics that the describe a cancer are of greater significance than the particular term used to describe a recognizable stage. The most widely used staging systems generally use at least one of the following characteristics for staging: the extent of tumor penetration into the colon wall, with greater penetration generally correlating with a more dangerous tumor; the extent of invasion of the tumor through the colon wall and into other neighboring tissues, with greater invasion generally correlating with a more dangerous tumor; the extent of invasion of the tumor into the regional lymph nodes, with greater invasion generally correlating with a more dangerous tumor; and the extent of metastatic invasion into more distant tissues, such as the liver, with greater metastatic invasion generally correlating with a more dangerous disease state. “Dukes A” and “Dukes B” colon cancers are neoplasias that have invaded into the wall of the colon but have not spread into other tissues. Dukes A colon cancers are cancers that have not invaded beyond the submucosa. Dukes B colon cancers are subdivided into two groups: “Dukes B1” and “Dukes B2”. “Dukes B1” colon cancers are neoplasias that have invaded up to but not through the muscularis propria. Dukes B2 colon cancers are cancers that have breached completely through the muscularis propria. Over a five year period, patients with Dukes A cancer who receive surgical treatment (i.e. removal of the affected tissue) have a greater than 90% survival rate. Over the same period, patients with Dukes B1 and Dukes B2 cancer receiving surgical treatment have a survival rate of about 85% and 75%, respectively. Dukes A, B1 and B2 cancers are also referred to as T1, T2 and T3-T4 cancers, respectively.

“Dukes C” colon cancers are cancers that have spread to the regional lymph nodes, such as the lymph nodes of the gut. Patients with Dukes C cancer who receive surgical treatment alone have a 35% survival rate over a five year period, but this survival rate is increased to 60% in patients that receive chemotherapy.

“Dukes D” colon cancers are cancers that have metastasized to other organs. The liver is the most common organ in which metastatic colon cancer is found. Patients with Dukes D colon cancer have a survival rate of less than 5% over a five year period, regardless of the treatment regimen.

As noted above, early detection of colon neoplasia, coupled with appropriate intervention, is important for increasing patient survival rates. Present systems for screening for colon neoplasia are deficient for a variety of reasons, including a lack of specificity or sensitivity (e.g. Fecal Occult Blood Test, flexible sigmoidoscopy) or a high cost and intensive use of medical resources (e.g. colonoscopy). Alternative systems for detection of colon neoplasia would be useful in a wide range of other clinical circumstances as well. For example, patients who receive surgical or pharmaceutical therapy for colon cancer may experience a relapse. It would be advantageous to have an alternative system for determining whether such patients have a recurrent or relapsed colon neoplasia. As a further example, an alternative diagnostic system would facilitate monitoring an increase, decrease or persistence of colon neoplasia in a patient known to have a colon neoplasia. A patient undergoing chemotherapy may be monitored to assess the effectiveness of the therapy.

Accordingly, in certain embodiments, the invention provides molecular markers that distinguish between cells that are not part of a colon neoplasia, referred to herein as “healthy cells”, and cells that are part of a colon neoplasia (e.g. an adenoma or a colon cancer), referred to herein as “colon neoplasia cells”. Certain molecular markers of the invention, including ColoUp1 and ColoUp2, are expressed at significantly higher levels in adenomas, Dukes A, Dukes B1, Dukes B2 and metastatic colon cancer of the liver (liver metastases) than in healthy colon tissue, healthy liver or healthy colon muscle. Certain molecular markers, including ColoUp1 and ColoUp2 are expressed at significantly higher levels in cell lines derived from colon cancer or cell lines engineered to imitate an aspect of a colon cancer cell. Particularly preferred molecular markers of the invention are markers that distinguish between healthy cells and cells of an adenoma. While not wishing to be bound to theory, it is contemplated that because adenomas are thought to be an obligate precursor for greater than 90% of colon cancers, markers that distinguish between healthy cells and cells of an adenoma are particularly valuable for screening apparently healthy patients to determine whether the patient is at increased risk for (predisposed to) developing a colon cancer. Furthermore, particularly preferred molecular markers are those that are actually present in the serum of an animal having a colon neoplasia, and in general, a secreted protein will generally occur in the serum only if it is secreted from a cell contacting a blood vessel, or a compartment in diffusional contact with a blood vessel. For example, protein secreted from a large or advanced colon cancer will generally be found in the blood stream, but a protein secreted from a colon adenoma may not be present in the blood unless it is secreted from the basolateral face of the cell. Molecular markers that occur in the urine are generally derived from a polypeptide that is present in the blood. Optionally, a molecular marker is one that is present in the lumen of the colon (e.g., may be found in the intestinal mucous or in stool samples), and such a marker will generally be one that is secreted from the apical face of a cell.

In certain embodiments, the invention provides methods for using ColoUp molecular markers for determining whether a patient has or does not have a condition characterized by increased expression of one or more ColoUp nucleic acids or proteins described herein. In certain embodiments, the invention provides methods for determining whether a patient is or is not likely to have a colon neoplasia. In further embodiments, the invention provides methods for determining whether the patient is having a relapse or determining whether a patient's colon neoplasia is responding to treatment.

3. Methods for Identifying Candidate Molecular Markers for Colon Neoplasia

In certain aspects, the invention relates to the observation that when gene expression data is analyzed using carefully selected criteria, the likelihood of identifying strong candidate molecular markers of a colon neoplasia is quite high. Accordingly, in certain embodiments, the invention provides methods and criteria for analyzing gene expression data to identify candidate molecular markers for colon neoplasia. Although methods and criteria of the invention may be applied to essentially any relevant gene expression data, the benefits of using the inventive methods and criteria are readily apparent when applied to the copious data produced by highly parallel gene expression measurement systems, such as microarray systems. The human genome is estimated to be capable of producing roughly 20,000 to 100,000 different gene transcripts, thousands of which may show a change in expression level in healthy cells versus colon neoplasia cells. It is relatively cost-effective to obtain large quantities of gene expression data and to use this data to identify thousands of candidate molecular markers. However, a significant amount of labor intensive experimentation is generally needed to move from the identification of a candidate molecular marker to an effective diagnostic test for a health condition of interest. In fact, as of the time of filing of this application, the resources required to generate a diagnostic test from a single candidate molecular marker identified by gene expression data are large enough that it is essentially impossible to extract commercially valuable and clinically useful diagnostics from a list of hundreds or thousands of genes whose expression levels change in a particular situation. Accordingly, there is a substantial practical value in being able to select a small number (e.g. ten or fewer) of high-quality molecular markers for further study.

In certain embodiments, candidate molecular markers for colon neoplasia may be selected by comparing gene expression in liver metastatic colon cancer samples (“liver mets”), normal (non-neoplastic) colon samples and normal liver samples. In this embodiment, candidate molecular markers are those genes (and their gene products) that have a level of expression in liver mets (assessed as a median expression level across the sample set) that is at least four times greater than the level of expression in normal colon samples (also assessed as a median expression level across the sample set). Furthermore, in this embodiment, the median level of expression in liver mets should be greater than the median level of expression in normal liver samples. The criteria employed in this embodiment provide a high threshold to eliminate most lower quality markers and further eliminate contaminants from liver tissue.

In certain embodiments, candidate molecular markers for colon neoplasia may be selected by comparing gene expression in normal colon to gene expression in a plurality of different cell lines cultured from metastatic colon cancer samples. For example median metastatic colon cancer cell line gene expression may be calculated as the median of 8 colon cancer cell lines of the Vaco colon cancer cell line series (Markowitz, S. et al. Science. 268: 1336-1338, 1995), such as the following liver metastatses-derived cell lines: V394, V576, V241, V9M, V400, V10M, V503, V786. In embodiments employing this criterion, candidate molecular markers are those genes (and their gene products) that have at least a three-fold higher median level of expression across the cell lines tested than in the normal colon tissue.

In certain embodiments, candidate molecular markers for colon neoplasia may be selected by comparing gene expression in normal colon to gene expression in a plurality of colon cancer xenografts grown in athymic mice (“xenografts”). In embodiments employing this criterion, candidate molecular markers are those genes (and their gene products) that have at least a four-fold higher median level of expression across the xenografts tested than in the normal colon tissue.

In certain embodiments, candidate molecular markers for colon neoplasia may be selected by comparing maximum gene expression in normal colon to minimum gene expression in liver mets. In these embodiments, candidate molecular markers are those genes (and their gene products) that have a minimum gene expression in liver mets that is at least equal to the maximum gene expression in normal colon. Furthermore, in this embodiment, the median level of expression in liver mets should be greater than the median level of expression in normal liver samples.

In a preferred embodiment, a list of candidate molecular markers for colon neoplasia is selected by first identifying a subset of genes having a four-fold greater median expression in liver mets that in normal colon and in normal liver. This subset is then further narrowed to a final list by identifying those genes that have a three-fold greater median expression across colon cancer cell lines than in normal colon. Optionally, a particularly preferred list may be generated by further selecting those genes having a minimum gene expression in liver mets that is greater than or equal to the maximum gene expression in normal colon. The gene products (e.g. proteins and nucleic acids) of the short list of genes generated in these preferred embodiments constitute a list of high-quality candidate molecular markers for colon cancer.

In another preferred embodiment, a list of candidate molecular markers for colon neoplasia is selected by first identifying a subset of genes having a four-fold greater median expression in liver mets that in normal colon and in normal liver. This subset is then further narrowed by identifying those genes that have a nine-fold greater median expression in liver mets than in normal colon. This subset is then further narrowed to a final list by identifying those genes that have a four-fold greater median expression across colon cancer cell lines than in normal colon. The gene products (e.g. proteins and nucleic acids) of the short list of genes generated in these preferred embodiments constitute a list of high-quality candidate molecular markers for colon cancer.

Depending on the nature of the intended use for the molecular marker it may be desirable to add further criteria to any of the preceding embodiments. In certain embodiments, the invention relates to candidate molecular markers for categorizing a patient as likely to have or not likely to have a colon neoplasia (including adenomas and colon cancers), and in these embodiments, a high-quality candidate molecular marker will be expressed from a gene having an increased expression in both adenomas and liver mets relative to normal colon, and preferably in other colon cancer stages, including Dukes A, Dukes B1, Dukes B2 and Dukes C. In certain embodiments the invention relates to candidate molecular markers for categorizing a patient as likely to have or not likely to have a colon cancer (including metastatic and non-metastatic forms), and in these embodiments, a high-quality candidate molecular marker will be expressed from a gene having an increased expression in liver mets relative to adenomas and normal colon, and preferably there will be elevated expression in other colon cancer stages, including Dukes A, Dukes B1, Dukes B2 and Dukes C. In certain embodiments, the invention relates to candidate molecular markers for categorizing a patient as likely or not likely to have a metastatic colon cancer, and in such embodiments, a comparison to gene expression in other colon neoplasias (e.g. adenomas, Dukes A, Dukes B1, Dukes B2, Dukes C), while potentially useful, is not necessary, although it is noted that expression in non-metastatic states may indicate that a candidate molecular marker is not of high quality for distinguishing metastatic colon cancer from non-metastatic states.

Furthermore, in those embodiments pertaining to molecular markers to be used for detection in a body fluid, such as blood, a high quality molecular marker will preferably be a secreted protein. In those embodiments pertaining to neoplasia identification or targeting, a high quality molecular marker will preferably be a protein with a portion adherent to and exposed on the extracellular surface of a neoplasia, such as a transmembrane protein with a significant extracellular portion.

Gene expression data may be gathered using one or more of the many known and appropriate techniques that, in view of this specification, may be selected to one of skill in the art. In certain preferred embodiments, gene expression data is gathered by a highly parallel system, meaning a system that allows simultaneous or near-simultaneous collection of expression data for one hundred or more gene transcripts. Exemplary highly parallel systems include probe arrays (“arrays”) that are often divided into microarrays and macroarrays, where microarrays have a much higher density of individual probe species per area. Arrays generally consist of a surface to which probes that correspond in sequence to gene products (e.g., cDNAs, mRNAs, oligonucleotides) are bound at known positions. The probes can be, e.g., a synthetic oligomer, a fill-length cDNA, a less-than full length cDNA, or a gene fragment. Usually a microarray will have probes corresponding to at least 100 gene products and more preferably, 500, 1000, 4000 or more. Probes may be small oligomers or larger polymers, and there may be a plurality of overlapping or non-overlapping probes for each transcript.

The nucleic acids to be contacted with the microarray may be prepared in a variety of ways. Methods for preparing total and poly(A)+RNA are well known and are described generally in Sambrook et al., supra. Labeled cDNA may be prepared from mRNA by oligo dT-primed or random-primed reverse transcription, both of which are well known in the art (see e.g., Klug and Berger, 1987, Methods Enzymol. 152:316-325). cDNAs may be labeled by incorporation of labeled nucleotides or by labeling after synthesis. Preferred labels are fluorescent labels.

Nucleic acid hybridization and wash conditions are chosen so that the population of labeled nucleic acids will specifically hybridize to appropriate, complementary probes affixed to the matrix. Optimal hybridization conditions will depend on the length (e.g., oligomer versus polynucleotide greater than 200 bases) and type (e.g., RNA, DNA, PNA) of labeled nucleic acids and immobilized polynucleotide or oligonucleotide. General parameters for specific (i.e., stringent) hybridization conditions for nucleic acids are described in Sambrook et al., supra, and in Ausubel et al., 1987, Current Protocols in Molecular Biology, Greene Publishing and Wiley-Interscience, New York, which is incorporated in its entirety for all purposes. Non-specific binding of the labeled nucleic acids to the array can be decreased by treating the array with a large quantity of non-specific DNA—a so-called “blocking” step.

Signals, such as fluorescent emissions for each location on an array are generally recorded, quantitated and analyzed using a variety of computer software. Signal for any one gene product may be normalized by a variety of different methods. Arrays preferably include control and reference probes. Control probes are nucleic acids which serve to indicate that the hybridization was effective. Reference probes allow the normalization of results from one experiment to another, and to compare multiple experiments on a quantitative level. Reference probes are typically chosen to correspond to genes that are expressed at a relatively constant level across different cell types and/or across different culture conditions. Exemplary reference nucleic acids include housekeeping genes of known expression levels, e.g., GAPDH, hexokinase and actin.

Following the data gathering operation, the data will typically be reported to a data analysis system. To facilitate data analysis, the data obtained by the reader from the device will typically be analyzed using a digital computer. Typically, the computer will be appropriately programmed for receipt and storage of the data from the device, as well as for analysis and reporting of the data gathered, e.g., subtraction of the background, deconvolution multi-color images, flagging or removing artifacts, verifying that controls have performed properly, normalizing the signals, interpreting fluorescence data to determine the amount of hybridized target, normalization of background and single base mismatch hybridizations, and the like. Various analysis methods that may be employed in such a data analysis system, or by a separate computer are described herein.

A number of methods for constructing or using arrays are described in the following references. Schena et al., 1995, Science 270:467-470; DeRisi et al., 1996, Nature Genetics 14:457-460; Shalon et al., 1996, Genome Res. 6:639-645; Schena et al., 1995, Proc. Natl. Acad. Sci. USA 93:10539-11286; Fodor et al., 1991, Science 251:767-773; Pease et al., 1994, Proc. Natl. Acad. Sci. USA 91:5022-5026; Lockhart et al., 1996, Nature Biotech 14:1675; U.S. Pat. Nos. 6,051,380; 6,083,697; 5,578,832; 5,599,695; 5,593,839; 5,631,734; 5,556,752; 5,510,270; EP No. 0 799 897; PCT No. WO 97/29212; PCT No. WO 97/27317; EP No. 0 785 280; PCT No. WO 97/02357; EP No. 0 728 520; EP No. 0 721 016; PCT No. WO 95/22058.

A variety of companies provide microarrays and software for extracting certain information from microarray data. Such companies include Affymetrix (Santa Clara, Calif.), GeneLogic (Gaithersburg, Md.) and Eos Biotechnology Inc. (South San Francisco, Calif.).

While the above discussion focuses on the use of arrays for the collection of gene expression data, such data may also be obtained through a variety of other methods, that, in view of this specification, are known to one of skill in the art. Such methods include the serial analysis of gene expression (SAGE) technique, first described in Velculescu et al. (1995) Science 270, 484-487. Reverse transcriptase—polymerase chain reaction (RT-PCR) may be used, and particularly in combination with fluorescent probe systems such as the Taqman™ fluorescent probe system. Numerous RT-PCR samples can be analyzed simultaneously by conducting parallel PCR amplification, e.g., by multiplex PCR. Further techniques include dotblot analysis and related methods (see, e.g., G. A. Beltz et al., in Methods in Enzymology, Vol. 100, Part B, R. Wu, L. Grossmam, K. Moldave, Eds., Academic Press, New York, Chapter 19, pp. 266-308, 1985), Northern blots and in situ hybridization (probing a tissue sample directly).

The quality and biological relevance of gene expression data will be significantly affected by the quality of the biological material used to obtain gene expression. In preferred embodiments, the methods described herein for identifying candidate molecular markers for colon neoplasia employ tissue samples obtained with appropriate consent from human patients and rapidly frozen. At a point prior to gene expression analysis, the tissue sample is preferably prepared by carefully dissecting away as much heterogeneous tissue as is possible with the available tools. In other words, for a colon cancer sample, adherent non-cancerous tissue should be dissected away, to the extent that it is possible. In preferred embodiments, healthy tissue is obtained from a subject that has a colon neoplasia but is tissue that is not directly entangled in a neoplasia.

Example 1, below, illustrates the operation of a method of selecting high-quality molecular markers, and the following markers were selected, using criteria disclosed herein, from microarray expression data: ColoUp1, ColoUp2, ColoUp3, ColoUp4, ColoUp5, ColoUp6, ColoUp7 and ColoUp8. In addition, osteopontin was identified as having expression characteristics very similar to those identified using the selection criteria. Further experimentation (see Examples) demonstrated that these molecular markers fall into four categories: “secreted” (ColoUp1, ColoUp2 and osteopontin), “transmembrane” (ColoUp3), “transcription factors” (ColoUp4, ColoUp5) and “other” (ColoUp6, ColoUp7, ColoUp8). Further experimentation also demonstrated that ColoUp1, ColoUp2, ColoUp3, ColoUp5 and ColoUp7 are, generally speaking, expressed at higher levels in a variety of colon neoplasias (adenomas, Dukes B tumors, Dukes C tumors and liver mets) than in healthy cells. In addition, further experimentation demonstrated that osteopontin is overexpressed in colon cancers (Dukes B, Dukes C and liver mets) relative to adenomas and normal colon.

In certain embodiments, a preferred molecular marker for use in a diagnostic test that employs a body fluid sample, such as a blood or urine sample, or an excreted sample material, such as stool, is a secreted protein, such as the secreted portion of a ColoUp1 protein, ColoUp2 protein or osteopontin protein.

In certain embodiments, a preferred molecular marker for a method that involves targeting or marking a colon neoplasia is a transmembrane protein, such as ColoUp3, and particularly the extracellular portion of ColoUp3. Transmembrane proteins are desirable for such methods because they are both anchored to the neoplastic cell and exposed to the extracellular surface.

In certain embodiments, a preferred molecular marker for use in a diagnostic test to distinguish subjects likely to have a colon neoplasia from those not likely to have a colon neoplasia is a gene product of the ColoUp1, ColoUp2, ColoUp3, ColoUp4 or ColoUp5 genes. Examples of suitable gene products include proteins, both secreted and not secreted and transcripts. In embodiments employing proteins that are not secreted, such as ColoUp3, ColoUp4 and ColoUp5, a preferred embodiment of the diagnostic test is a test for the presence of the protein or transcript in cells shed from the colon or colon neoplasia (which, in the case of metastases is not necessarily located in the colon) into a sample material, such as stool. In embodiments employing proteins that are secreted, such as ColoUp1 and ColoUp2, a preferred embodiment of the diagnostic test is a test for the presence of the protein in a body fluid, such as urine or blood or an excreted material, such as stool. It should be noted, however, that intracellular protein may be present in a body fluid if there is significant cell lysis or through some other process. Likewise, secreted proteins are likely to be adherent, even if at a relatively low level, to the cells in which they were produced.

In certain embodiments, a preferred molecular marker for distinguishing subjects having a colon cancer from those having an adenoma or a normal colon is gene product of the ColoUp6 and osteopontin genes. In embodiments preferably employing marker proteins that are secreted, such as a test using a body fluid sample, a preferred marker is a secreted osteopontin protein.

ColoUp1:

A human ColoUp1 nucleic acid sequence encodes a full-length protein of 1361 amino acids. SignalP V1.1 predicts that human ColoUp1 protein has an N-terminal signal peptide that is cleaved between either amino acids 30-31 (ATS-TV) or amino acids 33-34 (TVA-AG). Four potential glycosylation sites are identified in ColoUp1 protein. Further, ColoUp1 protein is predicted to have multiple serine, threonine, and tyrosine phosphorylation sites for kinases such as protein kinase C, cAMP- and cGMP-dependent protein kinases, casein kinase II, and tyrosine kinases. The ColoUp1 protein shares limited sequence homology to a human transmembrane protein 2 (See Scott et al. 2000 Gene 246:265-74). A mouse ColoUp1 homolog is identified in existing GenBank databases and is linked with mesoderm development (see Wines et al. 2001 Genomics. 88-98; GenBank entry AAG41062, AY007815 for the 1179 bp nucleic acid sequence entry, with 363/390 (93%) identities with human ColoUp1).

As demonstrated herein, ColoUp1 is secreted from both the basolateral and apical surfaces of intestinal cells.

ColoUp2:

The ColoUp2 nucleic acid sequence encodes a full-length protein of 755 amino acids. The application also discloses certain polymorphisms that have been observed, for example at nucleotide 113 GCC→ACC (Ala-Thr); nt 480 GAA→GGA (Glu-Gly); and at nt 2220 CAG→CGG (Gln-Arg). The sequence of ColoUp2 protein is similar to that of alpha 3 type VI collagen, isoform 2 precursor. In addition, a few domains are identified in the ColoUp2 protein such as a von Willebrand factor type A domain (vWF) and an EGF-like domain. The vWF domain is found in various plasma proteins such as some complement factors, the integrins, certain collagen, and other extracellular proteins. Proteins with vWF domains participate in numerous biological events which involve interaction with a large array of ligands, for example, cell adhesion, migration, homing, pattern formation, and signal transduction. The EGF-like domain consisting of about 30-40 amino acid residues has been found many proteins. The functional significance of EGF domains is not yet clear. However, a common feature is that these EGF-like repeats are found in the extracellular domain of membrane-bound proteins or in proteins known to be secreted.

As demonstrated herein, ColoUp2 is secreted from both the apical and basolateral surfaces of intestinal cells, and can be found in the blood in two different forms, a full-length secreted form and a C-terminal fragment (approximately 55 kDa).

Osteopontin:

The Osteopontin nucleic acid sequence encodes a full-length protein of 300 amino acids. Osteopontin is an acidic glycoprotein and is produced primarily by osteoclasts, macrophages, T-cells, kidneys, and vascular smooth muscle cells. As a cytokine, Osteopontin is known to contribute substantially to metastasis formation by various cancers. In addition, it contributes to macrophage homing and cellular immunity, mediates neovascularization, inhibits apoptosis, and maintains the homeostasis of free calcium (see a review, Weber G. F. 2001 Biochim Biophys Acta. 1552:61-85).

ColoUp3:

The ColoUp3 nucleic acid sequence encodes a full-length protein of 829 amino acids. ColoUp3 is referred to in the literature as P-cadherin (or cadherin 3, type 1). P-cadherin belongs to a cadherin family that includes E-cadherin and N-cadherin. P-cadherin is expressed in placenta and stratified squamous epithelia (see Shimoyama et al. 1989 J Cell Biol. 109:1787-94), but not in normal colon. P-cadherin null mice develop mammary gland hyperplasia, dysplasia, and abnormal lymphoid infiltration (see Radice et al. 1997 J Cell Biol. 139:1025-32), demonstrating that loss of normal P-cadherin expression leads to cellular and glandular abnormalities. It has been shown that P-cadherin is aberrantly expressed in inflamed and dysplastic colitic mucosa, with concomitant E-cadherin downregulation. Recently, aberrant P-cadherin expression is found as an early event in hyperplastic and dysplastic transformation in the colon (see Hardy et al. 2002 Gut. 50:513-514).

ColoUp4:

The ColoUp4 nucleic acid sequence encodes a full-length protein of 694 amino acids. ColoUp4 is referred to in the literature as NF-E2 related factor 3 (NRF3). NRF3 was identified and characterized as a novel Cap‘n’ collar (CNC) factor, with a basic region-leucine zipper domain highly homologous to those of other CNC proteins such as NRF1 and NRF2. These CNC factors bind to Maf recognition elements (MARE) through heterodimer formation with small Maf proteins In vitro and in vivo analyses showed that NRF3 can heterodimerize with MafK and that this complex binds to the MARE in the chicken β-globin enhancer and can activate transcription. NRF3 mRNA is highly expressed in human placenta and B cell and monocyte lineage. (see Kobayashi et al. 1999 J Biol Chem. 274:6443-52).

ColoUp5:

The ColoUp5 nucleic acid sequence encodes a full-length protein of 402 amino acids. ColoUp5 is referred to in the literature as FoxQ1 (Forkhead box, subclass q, member 1, formerly known as HFH-1). FoxQ1 is a member of the evolutionarily conserved winged helix/forkhead transcription factor gene family. The hallmark of this family is a conserved DNA binding region of approximately 110 amino acids (FOX domain). Members of the FOX gene family are found in a broad range of organisms from yeast to human. Human FoxQ1 gene is expressed in different tissues such as stomach, trachea, bladder, and salivary gland. FoxQ1 gene plays important roles in tissue-specific gene regulation and development, for example, embryonic development, cell cycle regulation, cell signaling, and tumorigenesis. The FoxQ1 gene is located on chromosome 6p23-25. Sequence analysis indicates that human FoxQ1 shows 82% homology with the mouse Foxq1 gene (formerly Hfh-1L) and with a revised sequence of the rat FoxQ1 gene (formerly Hfh-1). Mouse FoxQ1 was shown to regulate differentiation of hair in Satin mice. The DNA-binding motif (i.e., the FOX domain) is well conserved, showing 100% identity in human, mouse, and rat. The human FoxQ1 protein sequence contains two putative transcriptional activation domains, which share a high amino acid identity with the corresponding mouse and rat domains (see Bieller et al. 2001 DNA Cell Biol. 20:555-61).

ColoUp6:

The ColoUp6 nucleic acid sequence encodes a full-length protein of 209 amino acids. The ColoUp6 protein is 99% identical to the C-terminal portion of keratin 23 (or cytokeratin 23, or the type I intermediate filament cytokeratin), and accordingly the term ColoUp6 includes both the 209 amino acid protein (and related nucleic acids, fragments, variants, etc.) and the cytokeratin 23 amino acid sequence of GenBank entry BAA92054.1 (and related nucleic acids, fragments, variants, etc.). Keratin 23 mRNA was found highly induced in different pancreatic cancer cell lines in response to sodium butyrate. The keratin 23 protein has 422 amino acids, and has an intermediate filament signature sequence and extensive homology to type I keratins. It is suggested that keratin 23 is a novel member of the acidic keratin family that is induced in pancreatic cancer cells undergoing differentiation by a mechanism involving histone hyperacetylation (See Zhang et al. 2001 Genes Chromosomes Cancer. 30:123-35).

ColoUp7:

The ColoUp7 nucleic acid sequence is an EST sequence. No information relating to the function of the ColoUp7 gene is identified.

ColoUp8:

The ColoUp8 nucleic acid sequence encodes a full-length protein of 278 amino acids. No function has been suggested relating to the ColoUp8 gene.

Accordingly, in certain embodiments, the application provides isolated, purified or recombinant ColoUp1 , ColoUp2, ColoUp3, ColoUp4, ColoUp5, ColoUp6, ColoUp7, ColoUp8 and osteopontin nucleic acids. In certain embodiments, such nucleic acids may encode a complete or partial ColoUp polypeptide or such nucleic acids may also be probes or primers useful for methods involving detection or amplification of ColoUp nucleic acids. In certain embodiments, a ColoUp nucleic acid is single-stranded or double-stranded and composed of natural nucleic acids, nucleotide analogs, or mixtures thereof. In certain embodiments, the application provides isolated, purified or recombinant nucleic acids comprising a nucleic acid sequence that is at least 90% identical to a nucleic acid sequence of any of SEQ ID Nos: 3-12, or a complement thereof, and optionally at least 95%, 97%, 98%, 99%, 99.3%, 99.5%, 99.7% or 100% identical to a nucleic acid of any of SEQ ID Nos: 3-12, or a complement thereof. In certain preferred embodiments, the application provides a isolated, purified or recombinant nucleic acids comprising a nucleic acid sequence that is at least 90%, 95%, 97%, 98%, 99%, 99.3%, 99.5%, 99.7% or 100% identical to a nucleic acid of any of SEQ ID Nos: 3-12, or a complement thereof. In certain embodiments, the application provides isolated, purified or recombinant nucleic acids comprising a nucleic acid sequence that encodes a polypeptide that is at least 90% identical to an amino acid sequence of any of SEQ ID Nos: 1-3 or 13-21, or a complement thereof, and optionally at least 95%, 97%, 98%, 99%, 99.3%, 99.5%, 99.7% or 100% identical to an amino acid sequence of any of SEQ ID Nos: 1-3 or 13-21, or a complement thereof. In certain preferred embodiments, the application provides isolated, purified or recombinant nucleic acids comprising a nucleic acid sequence that encodes a polypeptide that is at least 90% identical to an amino acid sequence of any of SEQ ID Nos: 3, 14 or 21, or a complement thereof, and optionally at least 95%, 97%, 98%, 99%, 99.3%, 99.5%, 99.7% or 100% identical to an amino acid sequence of any of SEQ ID Nos: 3, 14 or 21, or a complement thereof.

In further embodiments, the application provides expression constructs, vectors and cells comprising a ColoUp nucleic acid. Expression constructs are nucleic acid constructs that are designed to permit expression of an expressible nucleic acid (e.g. a ColoUp nucleic acid) in a suitable cell type or in vitro expression system. A variety of expression construct systems are, in view of this specification, well known in the art, and such systems generally include a promoter that is operably linked to the expressible nucleic acid. The promoter may be a constitutive promoter, as in the case of many viral promoters, or the promoter may be a conditional promoter, as in the case of the prokaryotic lacI-repressible, IPTG-inducible promoter and as in the case of the eukaryotic tetracycline-inducible promoter. Vectors refer to any nucleic acid that is capable of transporting another nucleic acid to which it has been linked between different cells or viruses. One type of vector is an episome, i.e., a nucleic acid capable of extra-chromosomal replication, such as a plasmid. Episome-type vectors typically carry an origin of replication that directs replication of the vector in a host cell. Another type of vector is an integrative vector that is designed to recombine with the genetic material of a host cell. Vectors may be both autonomously replicating and integrative, and the properties of a vector may differ depending on the cellular context (i.e. a vector may be autonomously replicating in one host cell type and purely integrative in another host cell type). Vectors capable of directing the expression of genes to which they are operatively linked are referred to herein as “expression vectors”. Vectors that carry an expression construct are generally expression vectors. Vectors have been designed for a variety of cell types. For example, in the bacterium E. coli, commonly used vectors include pUC plasmids, pBR322 plasmids, pBlueScript and M13 plasmids. In insect cells (e.g. SF-9, SF-21 and High-Five cells), commonly used vectors include BacPak6 (Clontech) and BaculoGold (Pharmingen) (both Clontech and Pharmingen are divisions of Becton, Dickinson and Co., Franklin Lakes, N.J.). In mammalian cells (e.g. Chinese hamster ovary (CHO) cells, Vaco cells and human embryonic kidney (HEK) cells), commonly used vectors include pCMV vectors (Stratagene, Inc., La Jolla, Calif.), and pRK vectors. In certain embodiments, the application provides cells that comprise a ColoUp nucleic acid, particularly a recombinant ColoUp nucleic acid, such as an expression construct or vector that comprises a ColoUp nucleic acid. Cells may be eukaryotic or prolaryotic, depending on the anticipated use. Prokaryotic cells, especially E. coli, are particularly useful for storing and replicating nucleic acids, particularly nucleic acids carried on plasmid or viral vectors. Bacterial cells are also particularly useful for expressing nucleic acids to produce large quantities of recombinant protein, but bacterial cells do not usually mimic eukaryotic post-translational modifications, such as glycosylations or lipid-modifications, and so will tend to be less suitable for production of proteins in which the post-translational modification state is significant. Eukaryotic cells, and especially cell types such as insect cells that work with baculovirus-based protein expression systems, and Chinese hamster ovary cells, are good systems for expressing eukaryotic proteins that have significant post-translational modifications. Eukaryotic cells are also useful for studying various aspects of the function of eukaryotic proteins. For example, colon cancer cell lines are good model systems for studying the role of ColoUp genes and proteins in colon cancers.

In certain aspects the application further provides methods for preparing ColoUp polypeptides. In general, such methods comprise obtaining a cell that comprises a nucleic acid encoding a ColoUp polypeptide, and culturing the cell under conditions that cause production of the ColoUp polypeptide. Polypeptides produced in this manner may be obtained from the appropriate cell or culture fraction. For example, secreted proteins are most readily obtained from the culture supernatant, soluble intracellular proteins are most readily obtained from the soluble fraction of a cell lysate, and membrane proteins are most readily obtained from a membrane fraction. However, proteins of each type can generally be found in all three types of cell or culture fraction. Crude cellular or culture fractions may be subjected to further purification procedures to obtain substantially purified ColoUp polypeptides. Common purification procedures include affinity purification (e.g. with hexahistidine-tagged polypeptides), ion exchange chromatography, reverse phase chromatography, gel filtration chromatography, etc.

In certain aspects the application provides recombinant, isolated, substantially purified or purified ColoUp1, ColoUp2, ColoUp3, ColoUp4, ColoUp5, ColoUp6, ColoUp7, ColoUp8 and osteopontin polypeptides. In certain embodiments, such polypeptides may encode a complete or partial ColoUp polypeptide. In certain embodiments, a ColoUp polypeptide is composed of natural amino acids, amino acid analogs, or mixtures thereof. ColoUp polypeptides may also include one or more post-translational modifications, such as glycosylation, phosphorylation, lipid modification, acetylation, etc. In certain embodiments, the application provides isolated, substantially purified, purified or recombinant polypeptides comprising an amino acid sequence that is at least 90% identical to an amino acid sequence of any of SEQ ID Nos: 1-3 or 13-21 and optionally at least 95%, 97%, 98%, 99%, 99.3%, 99.5% or 99.7% identical to a nucleic acid of any of SEQ ID Nos: 1-3 or 13-21. In certain preferred embodiments, the application provides a isolated, substantially purified, purified or recombinant polypeptide comprising an amino acid sequence that is at least 90%, 95%, 97%, 98%, 99%, 99.3%, 99.5% or 99.7% identical to a nucleic acid of any of SEQ ID Nos: 3, 14 or 21. In certain preferred embodiments, the application provides an isolated, subtstantially purified, purified or recombinant polypeptide comprising an amino acid sequence that differs from SEQ ID Nos. 3, 14 or 21 by no more than 4 amino acid substitutions, additions or deletions. Optionally, a polypeptide of the invention comprises an additional moiety, such as an additional polypeptide sequence or other added compound, with a particular function, such as an epitope tag that facilitates detection of the recombinant polypeptide with an antibody, a purification moiety that facilitates purification (e.g. by affinity purification), a detection moiety, that facilitates detection of the polypeptide in vivo or in vitro, or an antigenic moiety that increases the antigenicity of the polypeptide so as to facilitate antibody production. Often, a single moiety will provide multiple functionalities. For example, an epitope tag will generally also assist in purification, because an antibody that recognizes the epitope can be used in an affinity purification procedure as well. Examples of commonly used epitope tags are: an HA tag, a hexahistidine tag, a V5 tag, a Glu-Glu tag, a c-myc tag, a VSV-G tag, a FLAG tag, an enterokinase cleavage site tag and a T7 tag. Commonly used purification moieties include: a hexahistidine tag, a glutathione-S-transferase domain, a cellulose binding domain and a biotin tag. Commonly used detection moieties include fluorescent proteins (e.g. green fluorescent proteins), a biotin tag, and chromogenic/fluorogenic enzymes (e.g. beta-galactosidase and luciferase). Commonly used antigenic moieties include the keyhole limpet hemocyanin and serum albumins. Note that these moieties need not be polypeptides and need not be connected to the polypeptide by a traditional peptide bond.

4. Antibodies and Uses Therefor

Another aspect of the invention pertains to an antibody specifically reactive with a ColoUp polypeptide, preferably antibodies that are specifically reactive with ColoUp polypeptides such as ColoUp1 and ColoUp2 polypeptides. For example, by using immunogens derived from a ColoUp polypeptide, e.g., based on the cDNA sequences, anti-protein/anti-peptide antisera or monoclonal antibodies can be made by standard protocols (See, for example, Antibodies: A Laboratory Manual ed. by Harlow and Lane (Cold Spring Harbor Press: 1988)). A mammal, such as a mouse, a hamster or rabbit can be immunized with an immunogenic form of the peptide (e.g., a ColoUp polypeptide or an antigenic fragment which is capable of eliciting an antibody response, or a fusion protein). Techniques for conferring immunogenicity on a protein or peptide include conjugation to carriers or other techniques well known in the art. An immunogenic portion of a ColoUp polypeptide can be administered in the presence of adjuvant. The progress of immunization can be monitored by detection of antibody titers in plasma or serum. Standard ELISA or other immunoassays can be used with the immunogen as antigen to assess the levels of antibodies. In a preferred embodiment, the subject antibodies are immunospecific for antigenic determinants of a ColoUp polypeptide of a mammal, e.g., antigenic determinants of a protein set forth in SEQ ID Nos: 1-3 and 13-21, more preferably SEQ ID Nos: 1-3 or 21.

In one embodiment, antibodies are specific for the secreted proteins as encoded by nucleic acid sequences as set forth in SEQ ID Nos: 4-5. In another embodiment, the antibodies are immunoreactive with one or more proteins having an amino acid sequence that is at least 80% identical to an amino acid sequence as set forth in SEQ ID Nos: 1-3 and 13-21, preferably SEQ ID Nos: 1-3 or 21. In other embodiments, an antibody is immunoreactive with one or more proteins having an amino acid sequence that is at least 85%, 90%, 95%, 98%, 99%, 99.3%, 99.5%, 99.7% identical or 100% identical to an amino acid sequence as set forth in SEQ ID Nos: 1-3 and 13-21. More preferably, the antibody is immunoreactive with one or more proteins having an amino acid sequence that is at least 85%, 90%, 95%, 98%, 99%, 99.3%, 99.5%, 99.7% or identical to an amino acid sequence as set forth in SEQ ID NOs: 1-3 or 21. In certain preferred embodiments, the invention provides an antibody that binds to an epitope including the C-terminal portion of the polypeptide of SEQ ID Nos: 3, 14 or 21. In certain preferred embodiments, the invention provides an antibody that binds to an epitope of a ColoUp2 polypeptide that is prevalent in the blood of an animal having a colon neoplasia, such SEQ ID No: 3 or 21.

Following immunization of an animal with an antigenic preparation of a ColoUp polypeptide, anti-ColoUp antisera can be obtained and, if desired, polyclonal anti-ColoUp antibodies can be isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a mammalian ColoUp polypeptide of the present invention and monoclonal antibodies isolated from a culture comprising such hybridoma cells. In one embodiment anti-human ColoUp antibodies specifically react with the protein encoded by a nucleic acid having SEQ ID Nos: 4-12; more preferably the antibodies specifically react with the protein encoded by a nucleic acid having SEQ ID Nos: 4 or 5, and preferably a secreted protein that is produced by the expression of a nucleic acid having a sequence of SEQ ID Nos: 4 or 5.

The term antibody as used herein is intended to include fragments thereof which are also specifically reactive with one of the subject ColoUp polypeptides. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for a ColoUp polypeptide conferred by at least one CDR region of the antibody. In preferred embodiments, the antibodies, the antibody further comprises a label attached thereto and able to be detected, (e.g., the label can be a radioisotope, fluorescent compound, enzyme or enzyme co-factor).

In certain preferred embodiments, an antibody of the invention is a monoclonal antibody, and in certain embodiments the invention makes available methods for generating novel antibodies. For example, a method for generating a monoclonal antibody that binds specifically to a ColoUp polypeptide, such as a ColoUp2 polypeptide may comprise administering to a mouse an amount of an immunogenic composition comprising the ColoUp2 polypeptide effective to stimulate a detectable immune response, obtaining antibody-producing cells (e.g. cells from the spleen) from the mouse and fusing the antibody-producing cells with myeloma cells to obtain antibody-producing hybridomas, and testing the antibody-producing hybridomas to identify a hybridoma that produces a monocolonal antibody that binds specifically to the ColoUp2 polypeptide. Once obtained, a hybridoma can be propagated in a cell culture, optionally in culture conditions where the hybridoma-derived cells produce the monoclonal antibody that binds specifically to the ColoUp2 polypeptide. The monoclonal antibody may be purified from the cell culture.

Anti-ColoUp antibodies can be used, e.g., to detect ColoUp polypeptides in biological samples and/or to monitor ColoUp polypeptide levels in an individual, for determining whether or not said patient is likely to develop colon cancer or is more likely to harbor colon adenomas, or allowing determination of the efficacy of a given treatment regimen for an individual afflicted with colon neoplasia, colon cancer, metastatic colon cancer and colon adenomas. The level of ColoUp polypeptide may be measured in a variety of sample types such as, for example, in cells, stools, and/or in bodily fluid, such as in whole blood samples, blood serum, blood plasma and urine. The adjective “specifically reactive with” as used in reference to an antibody is intended to mean, as is generally understood in the art, that the antibody is sufficiently selective between the antigen of interest (e.g. a ColoUp polypeptide) and other antigens that are not of interest that the antibody is useful for, at minimum, detecting the presence of the antigen of interest in a particular type of biological sample. In certain methods employing the antibody, a higher degree of specificity in binding may be desirable. For example, an antibody for use in detecting a low abundance protein of interest in the presence of one or more very high abundance protein that are not of interest may perform better if it has a higher degree of selectivity between the antigen of interest and other cross-reactants. Monoclonal antibodies generally have a greater tendency (as compared to polyclonal antibodies) to discriminate effectively between the desired antigens and cross-reacting polypeptides. In addition, an antibody that is effective at selectively identifying an antigen of interest in one type of biological sample (e.g. a stool sample) may not be as effective for selectively identifying the same antigen in a different type of biological sample (e.g. a blood sample). Likewise, an antibody that is effective at identifying an antigen of interest in a purified protein preparation that is devoid of other biological contaminants may not be as effective at identifying an antigen of interest in a crude biological sample, such as a blood or urine sample. Accordingly, in preferred embodiments, the application provides antibodies that have demonstrated specificity for an antigen of interest (particularly, although not limited to, a ColoUp1 or ColoUp2 polypeptide) in a sample type that is likely to be the sample type of choice for use of the antibody. In a particularly preferred embodiment, the application provides antibodies that bind specifically to a ColoUp1 or ColoUp2 polypeptide in a protein preparation from blood (optionally serum or plasma) from a patient that has a colon neoplasia or that bind specifically in a crude blood sample (optionally a crude serum or plasma sample).

One characteristic that influences the specificity of an antibody:antigen interaction is the affinity of the antibody for the antigen. Although the desired specificity may be reached with a range of different affinities, generally preferred antibodies will have an affinity (a dissociation constant) of about 10⁻⁶, 10⁻⁷, 10⁻⁸, 10⁻⁹ or less.

In addition, the techniques used to screen antibodies in order to identify a desirable antibody may influence the properties of the antibody obtained. For example, an antibody to be used for certain therapeutic purposes will preferably be able to target a particular cell type. Accordingly, to obtain antibodies of this type, it may be desirable to screen for antibodies that bind to cells that express the antigen of interest (e.g. by fluorescence activated cell sorting). Likewise, if an antibody is to be used for binding an antigen in solution, it may be desirable to test solution binding. A variety of different techniques are available for testing antibody:antigen interactions to identify particularly desirable antibodies. Such techniques include ELISAs, surface plasmon resonance binding assays (e.g. the Biacore binding assay, Bia-core AB, Uppsala, Sweden), sandwich assays (e.g. the paramagnetic bead system of IGEN International, Inc., Gaithersburg, Md.), western blots, immunoprecipitation assays and immunohistochemistry.

Another application of anti-ColoUp antibodies of the present invention is in the immunological screening of cDNA libraries constructed in expression vectors such as gt11, gt18-23, ZAP, and ORF8. Messenger libraries of this type, having coding sequences inserted in the correct reading frame and orientation, can produce fusion proteins. For instance, gt11 will produce fusion proteins whose amino termini consist of β-galactosidase amino acid sequences and whose carboxy termini consist of a foreign polypeptide. Antigenic epitopes of a ColoUp polypeptide, e.g., other orthologs of a particular protein or other paralogs from the same species, can then be detected with antibodies, as, for example, reacting nitrocellulose filters lifted from infected plates with the appropriate anti-ColoUp antibodies. Positive phage detected by this assay can then be isolated from the infected plate. Thus, the presence of ColoUp homologs can be detected and cloned from other animals, as can alternate isoforms (including splice variants) from humans.

5. Methods for Detecting Molecular Markers in a Patient

In certain embodiments, the invention provides methods for detecting molecular markers, such as proteins or nucleic acid transcripts of the ColoUp markers described herein. In certain embodiments, a method of the invention comprises providing a biological sample and probing the biological sample for the presence of a ColoUp marker. Information regarding the presence or absence of the ColoUp marker, and optionally the quantitative level of the ColoUp marker, may then be used to draw inferences about the nature of the biological sample and, if the biological sample was obtained from a subject, the health state of the subject.

Samples for use with the methods described herein may be essentially any biological material of interest. For example, a sample may be a tissue sample from a subject, a fluid sample from a subject, a solid or semi-solid sample from a subject, a primary cell culture or tissue culture of materials derived from a subject, cells from a cell line, or medium or other extracellular material from a cell or tissue culture, or a xenograft (meaning a sample of a colon cancer from a first subject, e.g. a human, that has been cultured in a second subject, e.g. an immunocompromised mouse). The term “sample” as used herein is intended to encompass both a biological material obtained directly from a subject (which may be described as the primary sample) as well as any manipulated forms or portions of a primary sample. For example, in certain embodiments, a preferred fluid sample is a blood sample. In this case, the term sample is intended to encompass not only the blood as obtained directly from the patient but also fractions of the blood, such as plasma, serum, cell fractions (e.g. platelets, erythrocytes, lymphocytes), protein preparations, nucleic acid preparations, etc. A sample may also be obtained by contacting a biological material with an exogenous liquid, resulting in the production of a lavage liquid containing some portion of the contacted biological material. Furthermore, the term “sample” is intended to encompass the primary sample after it has been mixed with one or more additive, such as preservatives, chelators, anti-clotting factors, etc. In certain embodiments, a fluid sample is a urine sample. In certain embodiments, a preferred solid or semi-solid sample is a stool sample. In certain embodiments, a preferred tissue sample is a biopsy from a tissue known to harbor or suspected of harboring a colon neoplasia. In certain embodiments, a preferred cell culture sample is a sample comprising cultured cells of a colon cancer cell line, such as a cell line cultured from a metastatic colon cancer tumor or a colon-derived cell line lacking a functional TGF-β, TGF-β receptor or TGF-β signaling pathway. A subject is preferably a human subject, but it is expected that the molecular markers disclosed herein, and particularly their homologs from other animals, are of similar utility in other animals. In certain embodiments, it may be possible to detect a marker directly in an organism without obtaining a separate portion of biological material. In such instances, the term sample is intended to encompass that portion of biological material that is contacted with a reagent or device involved in the detection process.

In certain embodiments, a method of the invention comprises detecting the presence of a ColoUp protein in a sample. Optionally, the method involves obtaining a quantitative measure of the ColoUp protein in the sample. In view of this specification, one of skill in the art will recognize a wide range of techniques that may be employed to detect and optionally quantitate the presence of a protein. In preferred embodiments, a ColoUp protein is detected with an antibody. Suitable antibodies are described in a separate section below. In many embodiments, an antibody-based detection assay involves bringing the sample and the antibody into contact so that the antibody has an opportunity to bind to proteins having the corresponding epitope. In many embodiments, an antibody-based detection assay also typically involves a system for detecting the presence of antibody-epitope complexes, thereby achieving a detection of the presence of the proteins having the corresponding epitope. Antibodies may be used in a variety of detection techniques, including enzyme-linked immunosorbent assays (ELISAs), immunoprecipitations, Western blots. Antibody-independent techniques for identifying a protein may also be employed. For example, mass spectroscopy, particularly coupled with liquid chromatography, permits detection and quantification of large numbers of proteins in a sample. Two-dimensional gel electrophoresis may also be used to identify proteins, and may be coupled with mass spectroscopy or other detection techniques, such as N-terminal protein sequencing. RNA aptamers with specific binding for the protein of interest may also be generated and used as a detection reagent.

In certain preferred embodiments, methods of the invention involve detection of a secreted form of a ColoUp protein or osteopontin, particularly ColoUp1 protein or ColoUp2 protein.

Samples should generally be prepared in a manner that is consistent with the detection system to be employed. For example, a sample to be used in a protein detection system should generally be prepared in the absence of proteases. Likewise, a sample to be used in a nucleic acid detection system should generally be prepared in the absence of nucleases. In many instances, a sample for use in an antibody-based detection system will not be subjected to substantial preparatory steps. For example, urine may be used directly, as may saliva and blood, although blood will, in certain preferred embodiments, be separated into fractions such as plasma and serum.

In certain embodiments, a method of the invention comprises detecting the presence of a ColoUp expressed nucleic acid, such as an mRNA, in a sample. Optionally, the method involves obtaining a quantitative measure of the ColoUp expressed nucleic acid in the sample. In view of this specification, one of skill in the art will recognize a wide range of techniques that may be employed to detect and optionally quantitate the presence of a nucleic acid. Nucleic acid detection systems generally involve preparing a purified nucleic acid fraction of a sample, and subjecting the sample to a direct detection assay or an amplification process followed by a detection assay. Amplification may be achieved, for example, by polymerase chain reaction (PCR), reverse transcriptase (RT) and coupled RT-PCR. Detection of a nucleic acid is generally accomplished by probing the purified nucleic acid fraction with a probe that hybridizes to the nucleic acid of interest, and in many instances detection involves an amplification as well. Northern blots, dot blots, microarrays, quantitative PCR and quantitative RT-PCR are all well known methods for detecting a nucleic acid in a sample.

In certain embodiments, the invention provides nucleic acid probes that bind specifically to a ColoUp nucleic acid. Such probes may be labeled with, for example, a fluorescent moiety, a radionuclide, an enzyme or an affinity tag such as a biotin moiety. For example, the TaqMan® system employs nucleic acid probes that are labeled in such a way that the fluorescent signal is quenched when the probe is free in solution and bright when the probe is incorporated into a larger nucleic acid.

In certain embodiments, the application provides methods for imaging a colon neoplasm by targeting antibodies to any one of the markers ColoUp1 through ColoUp8 or osetopontin described herein, more preferably the antibodies are targeted to ColoUp3. The markers described herein may be targeted using monoclonal antibodies which may be labeled with radioisotopes for clinical imaging of tumors or with toxic agents to destroy them.

In other embodiments, the application provides methods for administering a imaging agent comprising a targeting moiety and an active moiety. The targeting moiety may be an antibody, Fab, F(Ab)2, a single chain antibody or other binding agent that interacts with an epitope specified by a polypeptide sequence having an amino acid sequence as set forth in SEQ ID Nos: 1-3 and 13-21, preferably an epitope specified by SEQ ID No: 16. The active moiety may be a radioactive agent, such as: radioactive heavy metals such as iron chelates, radioactive chelates of gadolinium or manganese, positron emitters of oxygen, nitrogen, iron, carbon, or gallium, ⁴³K, ⁵²Fe, ⁵⁷Co ⁶⁷Cu ⁶⁷Ga, ⁶⁸Ga, ¹²³I, ¹²⁵I, ¹³¹I, ¹³²I, or ⁹⁹Tc. The imaging agent is administered in an amount effective for diagnostic use in a mammal such as a human and the localization and accumulation of the imaging agent is then detected. The localization and accumulation of the imaging agent may be detected by radioscintigraphy, nuclear magnetic resonance imaging, computed tomography or positron emission tomography.

Immunoscintigraphy using monoclonal antibodies directed at the ColoUp markers may be used to detect and/or diagnose colon neoplasia. For example, monoclonal antibodies against the ColoUp marker such as ColoUp3 labeled with ⁹⁹Technetium, ¹¹¹Indium, ¹²⁵Iodine-may be effectively used for such imaging. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, preferably 1-10 millicuries, most often 2-5 millicuries are administered. Thus, compositions according to the present invention useful as imaging agents comprising a targeting moiety conjugated to a radioactive moiety comprise 0.1-100 millicuries, in some embodiments preferably 1-10 millicuries, in some embodiments preferably 2-5 millicuries, in some embodiments more preferably 1-5 millicuries.

6. Additional Disclosure

Proteins described herein may be classified as secreted proteins, transmembrane proteins or intracellular proteins. ColoUp1 and ColoUp2 are secreted proteins; the secretion of which can be either constitutive or regulated. These proteins have a signal peptide or signal sequence that targets the molecule to the secretory pathway. Secreted proteins are involved in numerous physiological events; by virtue of their circulating nature, they often serve to transmit signals to various other cell types. The secreted protein may function in an autocrine manner (acting on the cell that secreted the factor), a paracrine manner (acting on cells in close proximity to the cell that secreted the factor) or an endocrine manner (acting on cells at a distance). Thus secreted molecules find use in modulating or altering numerous aspects of physiology. Screted proteins are particularly preferred as targets for diagnostic markers, e.g., for blood, plasma, serum, or stool tests.

Nucleic acids disclosed herein may be used in several ways. In a first embodiment, nucleic acid probes are made and attached to biochips to be used in screening and diagnostic methods, as outlined below, or for administration, e.g., for gene therapy, vaccine, and/or antisense applications. Alternatively, nucleic acids that include coding regions of can be put into expression vectors for the expression of the proteins, again for screening purposes or for administration to a patient.

In a preferred embodiment, nucleic acid probes to a nucleic acid disclosed herein (both the nucleic acid sequences outlined in the figures and/or the complements thereof) are made. The nucleic acid probes attached to the biochip are designed to be substantially complementary to the ColoUp nucleic acids, i.e. the target sequence (either the target sequence of the sample or to other probe sequences, e.g., in sandwich assays), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under appropriate reaction conditions, particularly high stringency conditions, as outlined herein.

A nucleic acid probe is generally single stranded but can be partially single and partially double stranded. The strandedness of the probe is dictated by the structure, composition, and properties of the target sequence. In general, the nucleic acid probes range from about 8 to about 100 bases long, with from about 10 to about 80 bases being preferred, and from about 30 to about 50 bases being particularly preferred. That is, generally complements of ORFs or whole genes are not used. In some embodiments, nucleic acids of lengths up to hundreds of bases can be used.

In a preferred embodiment, more than one probe per sequence is used, with either overlapping probes or probes to different sections of the target being used. That is, two, three, four or more probes, with three being preferred, are used to build in a redundancy for a particular target. The probes can be overlapping (i.e., have some sequence in common), or separate. In some cases, PCR primers may be used to amplify signal for higher sensitivity.

ColoUp nucleic acids may be used to make a variety of expression vectors to express proteins which can then be used in screening assays or to generate antibodies, as described below. Expression vectors and recombinant DNA technology are well known to those of skill in the art (see, e.g., Ausubel, supra, and Gene Expression Systems (Fernandez & Hoeffler, eds, 1999)) and are used to express proteins. The expression vectors may be either self-replicating extrachromosomal vectors or vectors which integrate into a host genome. Generally, these expression vectors include transcriptional and translational regulatory nucleic acid operably linked to the nucleic acid encoding the metastatic colorectal cancer protein. The term “control sequences” refers to DNA sequences used for the expression of an operably linked coding sequence in a particular host organism. Control sequences that are suitable for prokaryotes, e.g., include a promoter, optionally an operator sequence, and a ribosome binding site. Eukaryotic cells are known to utilize promoters, polyadenylation signals, and enhancers. Transcriptional and translational regulatory nucleic acid will generally be appropriate to the host cell used to express the protein. Numerous types of appropriate expression vectors, and suitable regulatory sequences are known in the art for a variety of host cells. In general, transcriptional and translational regulatory sequences may include, but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. Promoter sequences encode either constitutive or inducible promoters. The promoters may be either naturally occurring promoters or hybrid promoters. Hybrid promoters, which combine elements of more than one promoter, are also known in the art, and are useful in the present invention. In addition, an expression vector may comprise additional elements. For example, the expression vector may have two replication systems, thus allowing it to be maintained in two organisms, e.g., in mammalian or insect cells for expression and in a procaryotic host for cloning and amplification. Furthermore, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art. In addition, in a preferred embodiment, the expression vector contains a selectable marker gene to allow the selection of transformed host cells. Selection genes are well known in the art and will vary with the host cell used.

Proteins disclosed herein may be produced by culturing a host cell transformed with an expression vector containing nucleic acid encoding a metastatic colorectal cancer protein, under the appropriate conditions to induce or cause expression of the metastatic colorectal cancer protein. Conditions appropriate for protein expression will vary with the choice of the expression vector and the host cell, and will be easily ascertained by one skilled in the art through routine experimentation or optimization. For example, the use of constitutive promoters in the expression vector will require optimizing the growth and proliferation of the host cell, while the use of an inducible promoter requires the appropriate growth conditions for induction. In addition, in some embodiments, the timing of the harvest is important. For example, the baculoviral systems used in insect cell expression are lytic viruses, and thus harvest time selection can be crucial for product yield. Appropriate host cells include yeast, bacteria, archaebacteria, fungi, and insect and animal cells, including mammalian cells. Of particular interest are Saccharomyces cerevisiae and other yeasts, E. coli, Bacillus subtilis, Sf9 cells, C129 cells, 293 cells, Neurospora, BHK, CHO, COS, HeLa cells, HLVEC (human umbilical vein endothelial cells), THP1 cells (a macrophage cell line) and various other human cells and cell lines.

In one embodiment, the proteins are derivative or variant ColoUp proteins as compared to the wild-type sequence. That is, as outlined more fully below, the derivative peptide will often contain at least one amino acid substitution, deletion or insertion, with amino acid substitutions being particularly preferred. The amino acid substitution, insertion or deletion may occur at a particular residue within the peptide. Also included within one embodiment of ColoUp proteins of the present invention are amino acid sequence variants. These variants typically fall into one or more of three classes: substitutional, insertional or deletional variants. These variants ordinarily are prepared by site specific mutagenesis of nucleotides in the encoding DNA, using cassette or PCR mutagenesis or other techniques, to produce DNA encoding the variant, and thereafter expressing the DNA in recombinant cell culture as outlined above. However, variant protein fragments having up to about 100-150 residues may be prepared by in vitro synthesis. Amino acid sequence variants are characterized by the predetermined nature of the variation, a feature that sets them apart from naturally occurring allelic or interspecies variation of the metastatic colorectal cancer protein amino acid sequence. The variants typically exhibit the same qualitative biological activity as the naturally occurring analogue, although variants can also be selected which have modified characteristics as will be more fully outlined below.

While the site or region for introducing an amino acid sequence variation is often predetermined, the mutation per se need not be predetermined. For example, in order to optimize the performance of a mutation at a given site, random mutagenesis may be conducted at the target codon or region and the expressed variants screened for the optimal combination of desired activity. Techniques exist for making substitution mutations at predetermined sites in DNA having a known sequence, e.g., M13 primer mutagenesis and PCR mutagenesis. Screening of the mutants is done using assays of protein activities.

Amino acid substitutions are typically of single residues; insertions usually will be on the order of from about 1 to 20 amino acids, although considerably larger insertions may be occasionally tolerated. Deletions range from about 1 to about 20 residues, although in some cases deletions may be much larger.

Substitutions, deletions, insertions or any combination thereof may be used to arrive at a final derivative. Generally these changes are done on a few amino acids to minimize the alteration of the molecule. Larger changes may be tolerated in certain circumstances. When small alterations in the characteristics of a protein are desired, substitutions are generally made in accordance with the amino acid substitution chart provided in the definition section.

Variants typically exhibit the same qualitative biological activity and will elicit the same immune response as the naturally-occurring analog, although variants also are selected to modify the characteristics of the metastatic colorectal cancer proteins as needed. Alternatively, the variant may be designed or reorganized such that the biological activity of the metastatic colorectal cancer protein is altered. For example, glycosylation sites may be altered or removed.

When a ColoUp protein is to be used to generate antibodies, e.g., for immunotherapy or immunodiagnosis, the protein should share at least one epitope or determinant with the full length protein. By “epitope” or “determinant” herein is typically meant a portion of a protein which will generate and/or bind an antibody or T-cell receptor in the context of MHC. Thus, in most instances, antibodies made to a smaller metastatic colorectal cancer protein will be able to bind to the full-length protein, particularly linear epitopes. In a preferred embodiment, the epitope is unique; that is, antibodies generated to a unique epitope show little or no cross-reactivity.

Methods of preparing polyclonal antibodies are well known (e.g., Coligan, supra; and Harlow & Lane, supra). Polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid disclosed herein or fragment thereof or a fusion protein thereof. It may be useful to conjugate the immunizing agent to a protein known to be immunogenic in the mammal being immunized. Immunogenic proteins include, e.g., keyhole limpet hemocyanin, serum albumin, bovine thyroglobulin, and soybean trypsin inhibitor. Adjuvants include, e.g., Freund's complete adjuvant and MPL-TDM adjuvant (monophosphoryl Lipid A, synthetic trehalose dicorynomycolate). The immunization protocol may be selected by one skilled in the art.

The antibodies may, alternatively, be monoclonal antibodies. Monoclonal antibodies may be prepared using hybridoma methods, such as those described by Kohler & Milstein, Nature 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by a nucleic acid of disclosed herein, or fragment thereof, or a fusion protein thereof Generally, either peripheral blood lymphocytes (“PBLs”) are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, pp. 59-103 (1986)). Immortalized cell lines are usually transformed mammalian cells, particularly myeloma cells of rodent, bovine and primate origin. Usually, rat or mouse myeloma cell lines are employed. The hybridoma cells may be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

In one embodiment, the antibodies are bispecific antibodies. Bispecific antibodies are typically monoclonal, preferably human or humanized, antibodies that have binding specificities for at least two different antigens or that have binding specificities for two epitopes on the same antigen. In one embodiment, one of the binding specificities is for a protein encoded by a nucleic acid disclosed herein or a fragment thereof, the other one is for any other antigen, and preferably for a cell-surface protein or receptor or receptor subunit, preferably one that is tumor specific. Alternatively, tetramer-type technology may create multivalent reagents.

In a preferred embodiment, the antibodies to a protein disclosed herein are capable of reducing or eliminating a biological function of the protein. That is, the addition of antibodies (either polyclonal or preferably monoclonal) to metastatic colorectal cancer tissue (or cells containing metastatic colorectal cancer) may reduce or eliminate the metastatic colorectal cancer. Generally, at least a 25% decrease in activity, growth, size or the like is preferred, with at least about 50% being particularly preferred and about a 95-100% decrease being especially preferred.

In a preferred embodiment the antibodies are humanized antibodies (e.g., Xenerex Biosciences, Mederex, Inc., Abgenix, Inc., Protein Design Labs, Inc.) Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

Human-like antibodies can also be produced using various techniques known in the art, including phage display libraries (Hoogenboom & Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, p. 77 (1985) and Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in virtually all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995).

By immunotherapy is meant treatment of metastatic colorectal cancer with an antibody raised against a protein disclosed herein. As used herein, immunotherapy can be passive or active. Passive immunotherapy as defined herein is the passive transfer of antibody to a recipient (patient). Active immunization is the induction of antibody and/or T-cell responses in a recipient (patient). Induction of an immune response is the result of providing the recipient with an antigen to which antibodies are raised. The antigen may be provided by injecting a polypeptide against which antibodies are desired to be raised into a recipient, or contacting the recipient with a nucleic acid capable of expressing the antigen and under conditions for expression of the antigen, leading to an immune response.

In a preferred embodiment the proteins against which antibodies are raised are secreted proteins as described above. Without being bound by theory, antibodies used for treatment, bind and prevent the secreted protein from binding to its receptor, thereby inactivating the secreted protein. In one aspect, the RNA expression levels of genes are determined for different cellular states in the metastatic colorectal cancer phenotype. Expression levels of genes in normal tissue (i.e., not undergoing metastatic colorectal cancer) and in metastatic colorectal cancer tissue (and in some cases, for varying severities of metastatic colorectal cancer that relate to prognosis, as outlined below) are evaluated to provide expression profiles. An expression profile of a particular cell state or point of development is essentially a “fingerprint” of the state. While two states may have any particular gene similarly expressed, the evaluation of a number of genes simultaneously allows the generation of a gene expression profile that is reflective of the state of the cell. By comparing expression profiles of cells in different states, information regarding which genes are important (including both up- and down-regulation of genes) in each of these states is obtained. Then, diagnosis may be performed or confirmed to determine whether a tissue sample has the gene expression profile of normal or cancerous tissue. This will provide for molecular diagnosis of related conditions.

In a preferred embodiment proteins described herein are used in drug screening assays. The proteins, antibodies, nucleic acids, modified proteins and cells are used in drug screening assays or by evaluating the effect of drug candidates on a “gene expression profile” or expression profile of polypeptides. In a preferred embodiment, the expression profiles are used, preferably in conjunction with high throughput screening techniques to allow monitoring for expression profile genes after treatment with a candidate agent (e.g., Zlokarnik, et al., Science 279:84-8 (1998); Heid, Genome Res 6:986-94, 1996).

In a preferred embodiment, the proteins, antibodies, nucleic acids, modified proteins and cells containing the native or modified proteins are used in screening assays. That is, the present invention provides novel methods for screening for compositions which modulate the metastatic colorectal cancer phenotype or an identified physiological function of a ColoUp protein. As above, this can be done on an individual gene level or by evaluating the effect of drug candidates on a “gene expression profile”. In a preferred embodiment, the expression profiles are used, preferably in conjunction with high throughput screening techniques to allow monitoring for expression profile genes after treatment with a candidate agent, see Zlokarnik, supra.

Having identified the differentially expressed genes herein, a variety of assays may be applied. In a preferred embodiment, assays may be run on an individual gene or protein level. That is, having identified a particular gene with altered regulation in metastatic colorectal cancer, test compounds can be screened for the ability to modulate gene expression or for binding to the ColoUp protein. “Modulation” thus includes an increase or a decrease in gene expression. The preferred amount of modulation will depend on the original change of the gene expression in normal versus tissue undergoing metastatic colorectal cancer, with changes of at least 10%, preferably 50%, more preferably 100-300%, and in some embodiments 300-1000% or greater. Thus, if a gene exhibits a 4-fold increase in metastatic colorectal cancer tissue compared to normal tissue, a decrease of about four-fold is often desired; similarly, a 10-fold decrease in metastatic colorectal cancer tissue compared to normal tissue often provides a target value of a 10-fold increase in expression to be induced by the test compound.

The amount of gene expression may be monitored using nucleic acid probes and the quantification of gene expression levels, or, alternatively, the gene product itself can be monitored, e.g., through the use of antibodies to the ColoUp protein and standard immunoassays. Proteomics and separation techniques may also allow quantification of expression. In a preferred embodiment, gene or protein expression monitoring of a number of entities, i.e., an expression profile, is monitored simultaneously. Such profiles will typically involve a plurality of those entities described herein. In this embodiment, the nucleic acid probes are attached to biochips as outlined herein for the detection and quantification of sequences in a particular cell. Alternatively, PCR may be used. Thus, a series, e.g., of microtiter plate, may be used with dispensed primers in desired wells. A PCR reaction can then be performed and analyzed for each well.

Expression monitoring can be performed to identify compounds that modify the expression of one or more ColoUp sequences disclosed herein. Generally, in a preferred embodiment, a test compound is added to the cells prior to analysis. Moreover, screens are also provided to identify agents that modulate metastatic colorectal cancer, modulate ColoUp proteins, bind to ColoUp protein, or interfere with the binding of a ColoUp protein and an antibody, substrate, or other binding partner.

In certain embodiments, combinatorial libraries of potential modulators will be screened for an ability to bind to a ColoUp polypeptide or to modulate activity. Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis. In one preferred embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics. A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)). The assays to identify modulators are amenable to high throughput screening. Preferred assays thus detect modulation of ColoUp gene transcription, polypeptide expression, and polypeptide activity. High throughput assays for evaluating the presence, absence, quantification, or other properties of particular nucleic acids or protein products are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding. In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, e.g., Zymark Corp. provides technical bulletins describing screening systems for detecting the modulation of gene transcription, ligand binding, and the like.

In one embodiment, modulators are proteins, often naturally occurring proteins or fragments of naturally occurring proteins. Thus, e.g., cellular extracts containing proteins, or random or directed digests of proteinaceous cellular extracts, may be used. In this way libraries of proteins may be made for screening in the methods of the invention. Particularly preferred in this embodiment are libraries of bacterial, fungal, viral, and mammalian proteins, with the latter being preferred, and human proteins being especially preferred. Particularly useful test compound will be directed to the class of proteins to which the target belongs, e.g., substrates for enzymes or ligands and receptors. In a preferred embodiment, the candidate compounds are organic chemical moieties, a wide variety of which are available in the literature.

After a candidate agent has been added and the cells allowed to incubate for some period of time, the sample containing a target sequence is analyzed. If required, the target sequence is prepared using known techniques. For example, the sample may be treated to lyse the cells, using known lysis buffers, electroporation, etc., with purification and/or amplification such as PCR performed as appropriate. For example, an in vitro transcription with labels covalently attached to the nucleotides is performed. Generally, the nucleic acids are labeled with biotin-FITC or PE, or with cy3 or cy5.

In a preferred embodiment, the target sequence is labeled with, e.g., a fluorescent, a chemiluminescent, a chemical, or a radioactive signal, to provide a means of detecting the target sequence's specific binding to a probe. The label also can be an enzyme, such as, alkaline phosphatase or horseradish peroxidase, which when provided with an appropriate substrate produces a product that can be detected. Alternatively, the label can be a labeled compound or small molecule, such as an enzyme inhibitor, that binds but is not catalyzed or altered by the enzyme. The label also can be a moiety or compound, such as, an epitope tag or biotin which specifically binds to streptavidin. For the example of biotin, the streptavidin is labeled as described above, thereby, providing a detectable signal for the bound target sequence. Unbound labeled streptavidin is typically removed prior to analysis.

The reactions outlined herein may be accomplished in a variety of ways. In one embodiment, the binding of the test compound is determined by competitive binding assay. The competitor is a binding moiety known to bind to the target molecule, such as an antibody, peptide, binding partner, ligand, etc. Under certain circumstances, there may be competitive binding between the compound and the binding moiety, with the binding moiety displacing the compound. In one embodiment, the test compound is labeled. Either the compound, or the competitor, or both, is added first to the protein for a time sufficient to allow binding, if present. Incubations may be performed at a temperature which facilitates optimal activity, typically between 4 and 40° C. Incubation periods are typically optimized, e.g., to facilitate rapid high throughput screening. Typically between 0.1 and 1 hour will be sufficient. Excess reagent is generally removed or washed away. The second component is then added, and the presence or absence of the labeled component is followed, to indicate binding.

In a preferred embodiment, the competitor is added first, followed by the test compound. Displacement of the competitor is an indication that the test compound is binding to the ColoUp protein and thus is capable of binding to, and potentially modulating, the activity of the ColoUp protein. In this embodiment, either component can be labeled. Thus, e.g., if the competitor is labeled, the presence of label in the wash solution indicates displacement by the agent. Alternatively, if the test compound is labeled, the presence of the label on the support indicates displacement.

In an alternative embodiment, the test compound is added first, with incubation and washing, followed by the competitor. The absence of binding by the competitor may indicate that the test compound is bound to ColoUp protein with a higher affinity. Thus, if the test compound is labeled, the presence of the label on the support, coupled with a lack of competitor binding, may indicate that the test compound is capable of binding to the protein.

Exemplification

The invention now being generally described, it will be more readily understood by reference to the following examples, which are included merely for purposes of illustration of certain aspects and embodiments of the present invention, and are not intended to limit the invention.

### EXAMPLE 1

### Selection of Eight Molecular Markers for Colon Neoplasia

Expression micro-array profiling was used to find genes whose expression was different between normal colon and metastatic colon cancer. Normal colon and metastatic colon cancer samples were analyzed for gene expression using DNA expression microarray techniques that profiled expression patterns of nearly 50,000 genes, ESTs and predicted exons. Analysis of the data identified eight molecular markers for colon neoplasia, as shown in Table 2.

TABLE 2
Eight Selected Molecular Markers for Colon Neoplasia

| | | (Median | (Median | | (Median | (Median |
|---|---|---|---|---|---|---|
| | | Liver | Liver | (Minimum | Met Cell | Met |
| | Example | Mets)/ | Mets)/ | Liver Mets)/ | Lines)/ | Xenografts)/ |
| | Sequences | (Median | (Median | (Maximum | (Median | Median |
| | (SEQ ID | Normal | Normal | Normal | Normal | Normal |
| Marker Name | Nos.) | Colon) | Liver) | Colon) | Colon) | Colon) |
| ColoUp1 | 1, 2, 4, 13 | 13.94 | 13.94 | 0.26 | 14.08 | 15.48 |
| ColoUp2 | 3, 5, 14 | 5.70 | 5.70 | 1.00 | 5.32 | 1.24 |
| ColoUp3 | 7, 16 | 16.36 | 16.36 | 0.80 | 21.50 | 15.68 |
| ColoUp4 | 8, 17 | 4.68 | 4.68 | 1.00 | 4.88 | 1.56 |
| ColoUp5 | 9, 18 | 4.58 | 4.74 | 1.15 | 4.82 | 4.63 |
| ColoUp6 | 10, 19 | 9.52 | 9.52 | 0.52 | 11.58 | 1.92 |
| ColoUp7 | 11 | 9.20 | 9.20 | 0.18 | 4.30 | 9.00 |
| ColoUp8 | 12, 20 | 4.78 | 4.78 | 1.27 | 3.76 | 2.72 |

Osteopontin was also identified as a molecular marker having similar characteristics (Example sequences SEQ ID Nos: 6, 15). Each of these molecular markers was subjected to additional analysis in various types of colon neoplasia. In the case of ColoUp1 and ColoUp2, the microarray expression was confirmed by Northern blot and secretion of the protein was established.

Data shown in Table 21 of priority application 10/087080 shows overexpression in the gene probe set numbered 409041 in colon cancer cells, which corresponds to SEQ ID No. 16 in that document and corresponds to ColoUp1 in the present application.

### EXAMPLE 2

### Expression Pattern of ColoUp1 in Various Cell Types

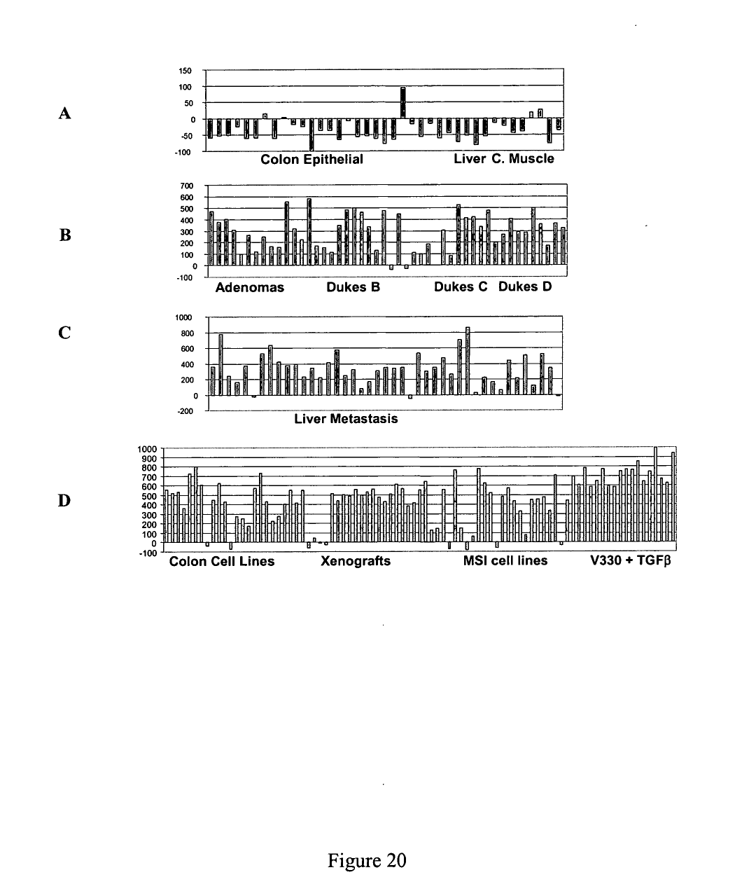

Shown in FIG. 20 is a graphical display of ColoUp1 expression levels measured for different tissue samples. ColoUp1 transcript was essentially undetectable (AI expression levels less than 0) in normal colon epithelial strips (labeled colon epithelial), in normal liver and in colonic muscle (labeled c. muscle). In contrast ColoUp1 expression was clearly detected in premalignant colon adenomas as well as in 90% of Dukes stage B (early node negative colon cancers), Dukes stage C (node positive colon cancer), Dukes stage D (primary colon cancers with associated metastatic spread) and in colon cancer liver metastasis (labeled liver metastasis). ColoUp1 expression was also demonstrated in colon cancer cell lines (labeled colon cell lines) and in colon cancer xenografts grown in athymic mice (labeled xenografts). The expression in cell lines and xenografts confirms that colon neoplasia cells are the source of ColoUp1 expression in the tumors.

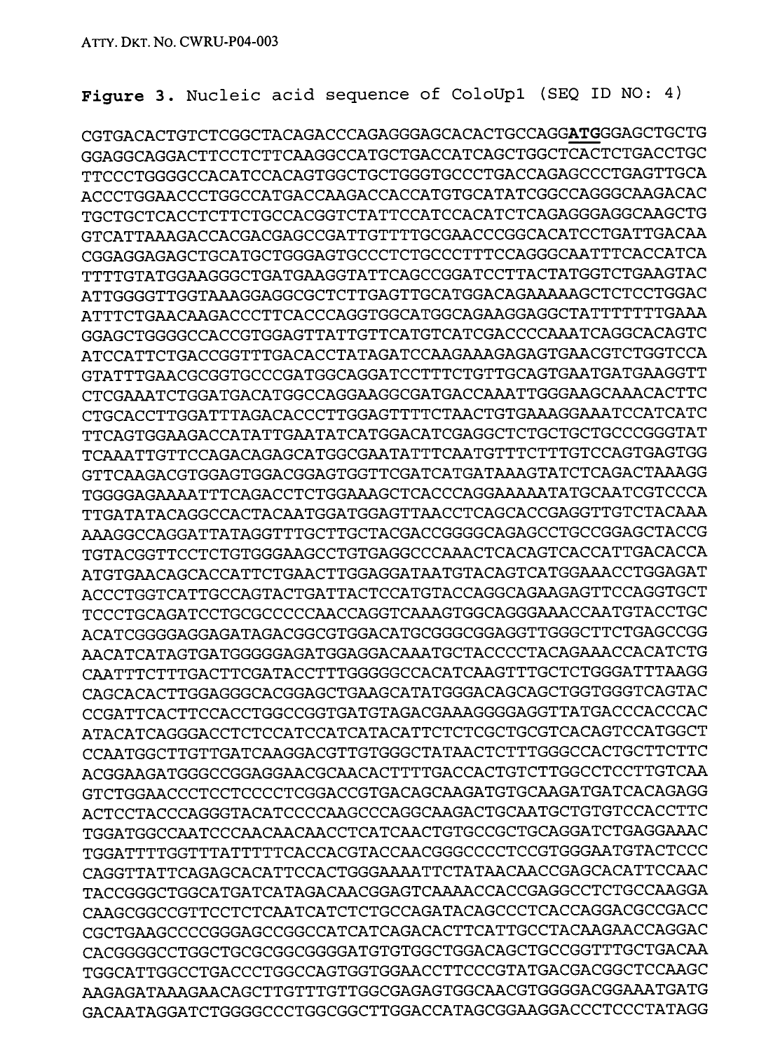

The probe for ColoUp1 was designed to recognize transcripts corresponding to gene KIAA1199, Genbank entry AB033025, Unigene entry Hs.50081. A transcript corresponding to this gene was amplified by RT-PCR from colon cancer cell line Vaco-394. The sequence of this transcript is presented in FIG. 3.

### EXAMPLE 3

### Confirmed Gene Expression Pattern of ColoUp1

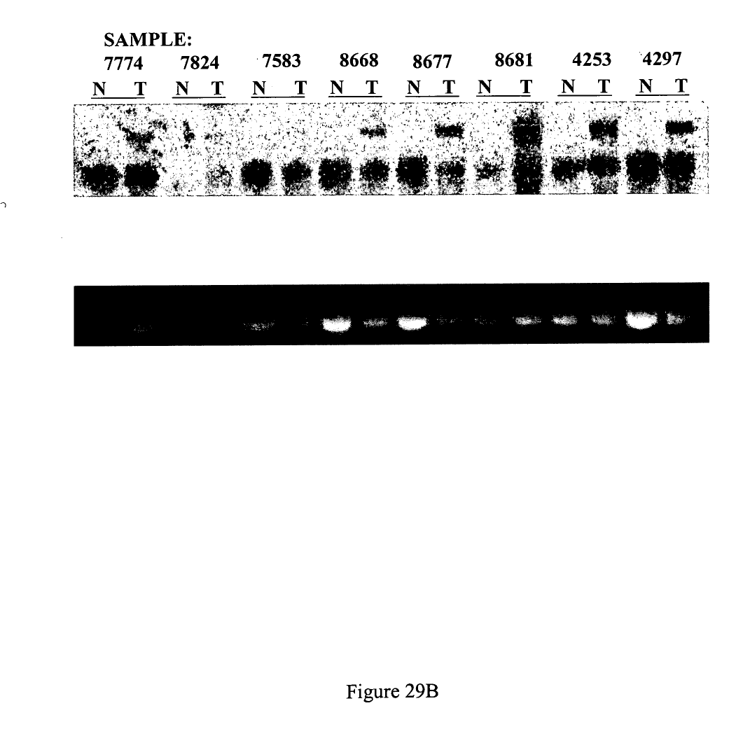

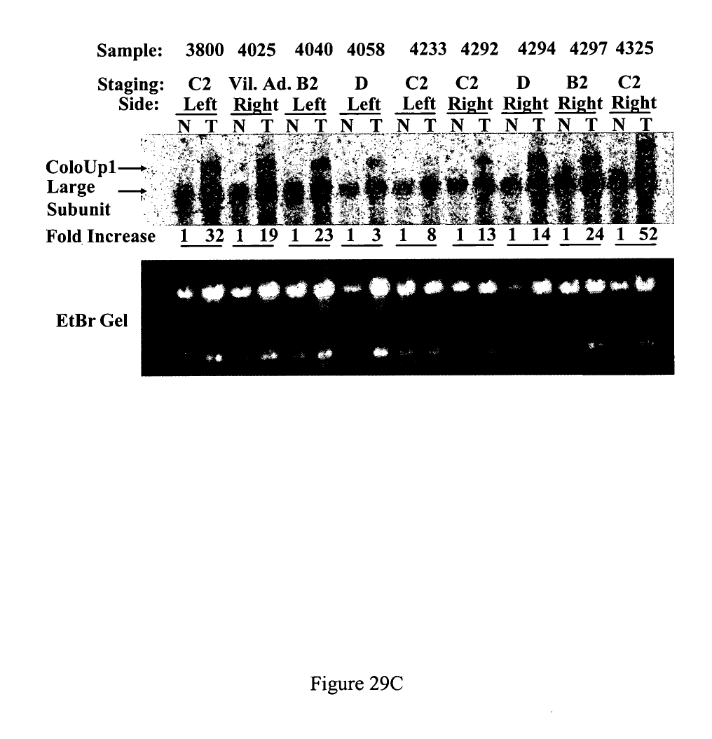

FIG. 29 shows a northern analysis using the cloned ColoUp1 cDNA that identifies a transcript running above the large ribosomal subunit (to which the probe cross hybridizes) that is not expressed in normal colon tissue samples and is ubiquitously expressed in a group of colon cancer cell lines.

FIGS. 29B and 29C show the results of northern analysis of ColoUp1 in normal colon tissue and colon neoplasms from 15 individuals with colon cancers and one individual with a colon adenoma. No normal colon sample expresses ColoUp1. However, expression is see in 13 of 15 colon cancers, and in the one colon adenoma. Expression is seen in cancers arising in both the right and left colon, and in cancers of Dukes Stage B2, C and D.

### EXAMPLE 4

### ColoUp1 is a Secreted Protein

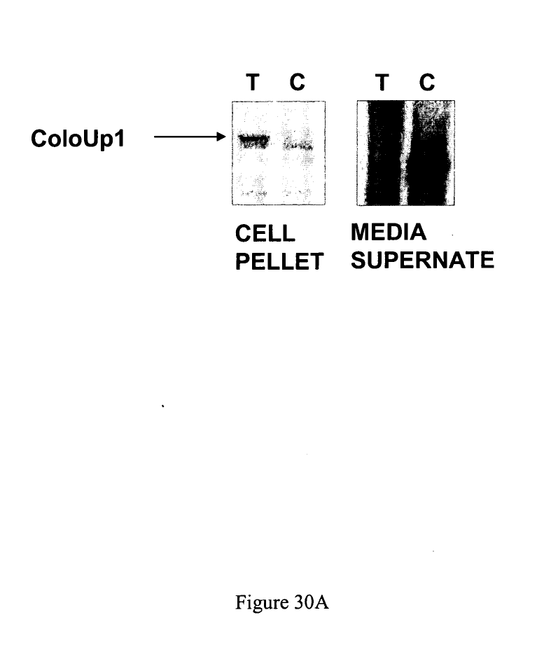

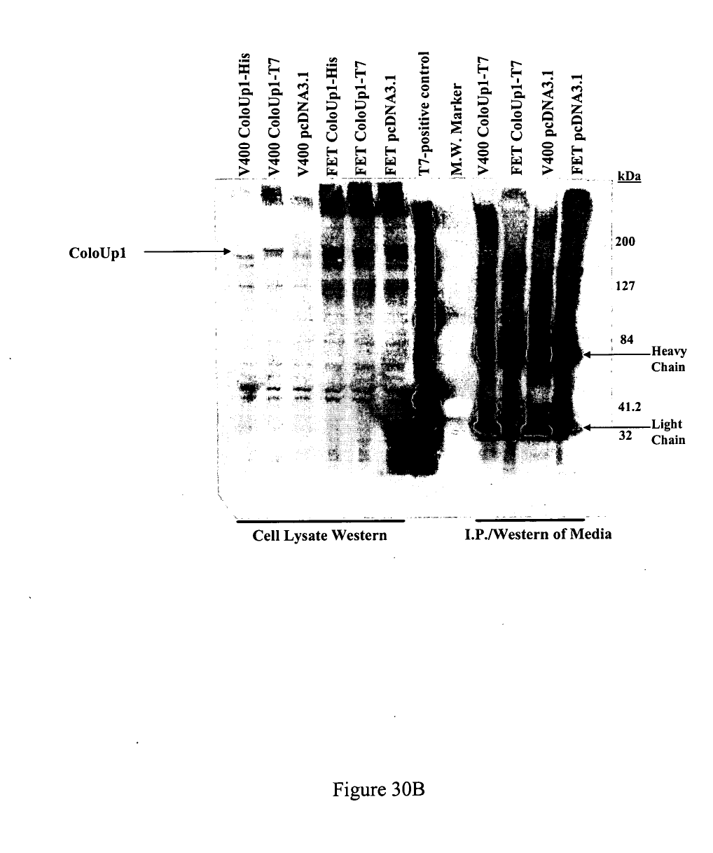

The cloned ColoUp1 colonic transcript was inserted into a cDNA expression vector with a C-terminal T7 epitope tag. FIG. 30A shows a summary of the behavior of the tagged protein expressed by transfection of the vector into Vaco400 cells. An anti T7 western blot shows expression of the transfected tagged protein detected in the lysate of a pellet of transfected cells (lane T of cell pellet) which is absent in cells transfected with a control empty expression vector (lane C of cell pellet). Moreover, serial immunoprecipitation and western blotting of T7 tagged protein from media in which V400 cells were growing (which had been clarified by centrifugation prior to immunoprecipatation) also clearly demonstrates secretion of ColoUp1 protein into the growth medium.

FIG. 30B shows the full gels demonstrating expression of tagged 409041 protein in V400 cells demonstrated by western analysis at left and shows detection of secreted 409041 protein in growth media as detected at right by serial immunoprecipitation and western analysis. (Antibody from the high level of serum in which FET cells are grown blocked the ability of staphA conjugated beads to precipitate anti-T7 bound to 409041 in growth media from FET cells).

### EXAMPLE 5

### Expression Pattern of ColoUp2 in Various Cell Types

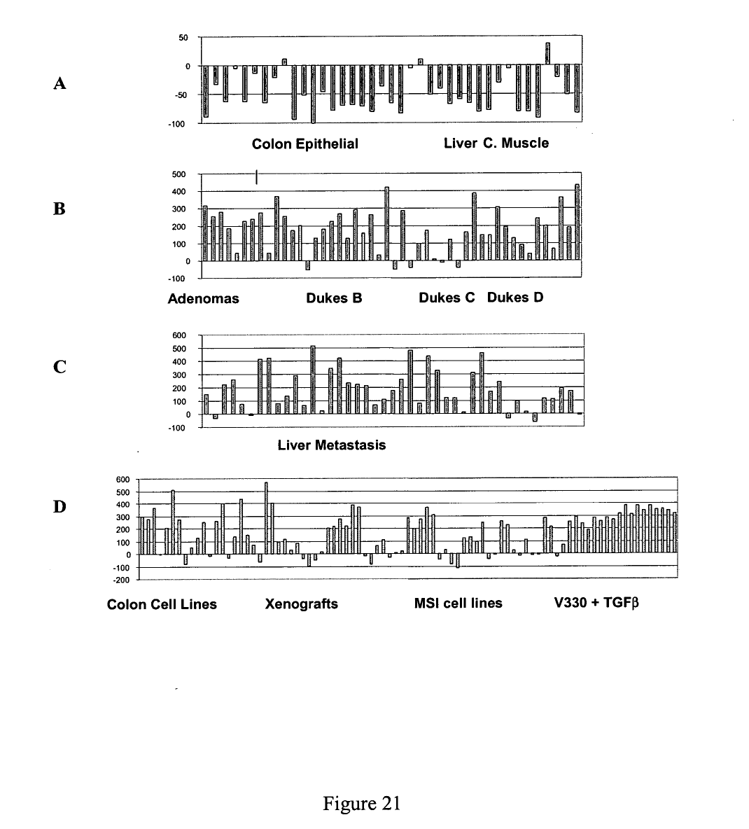

Shown in FIG. 21 is the graphical display of ColoUp2 expression levels measured for different samples analyzed. ColoUp2 transcript was essentially undetectable (AI expression levels less than 0) in normal colon epithelial strips (labeled colon epithelial), in normal liver and in colonic muscle (labeled c. muscle). In contrast ColoUp2 expression was clearly detected in premalignant colon adenomas as well as in 90% of Dukes stage B (early node negative colon cancers), Dukes stage C (node positive colon cancer), Dukes stage D (primary colon cancers with associated metastatic spread) and in colon cancer liver metastasis (labeled liver metastasis). ColoUp2 expression was also demonstrated in colon cancer cell lines (labeled colon cell lines) and in colon cancer xenografts grown in athymic mice (labeled xenografts). The expression in cell lines and xenografts confirms that colon neoplasia cells are the source of ColoUp2 expression in the tumors.

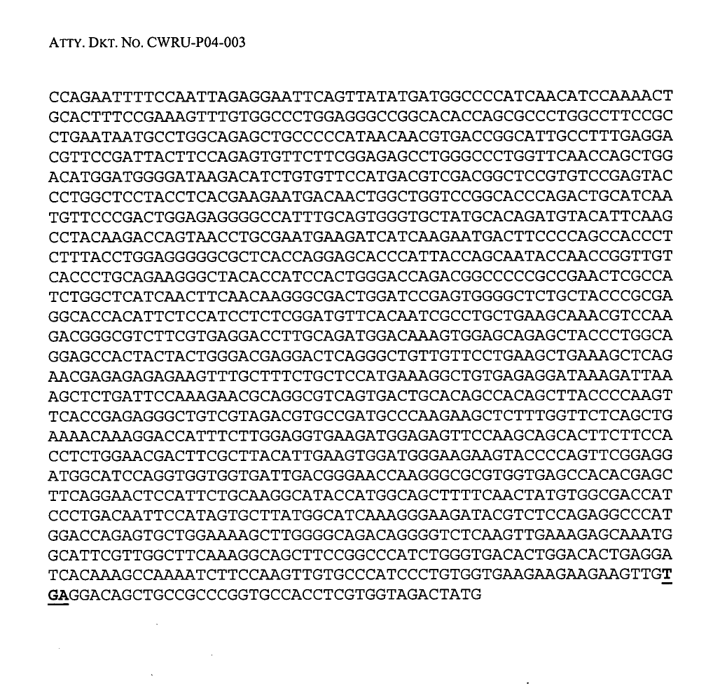

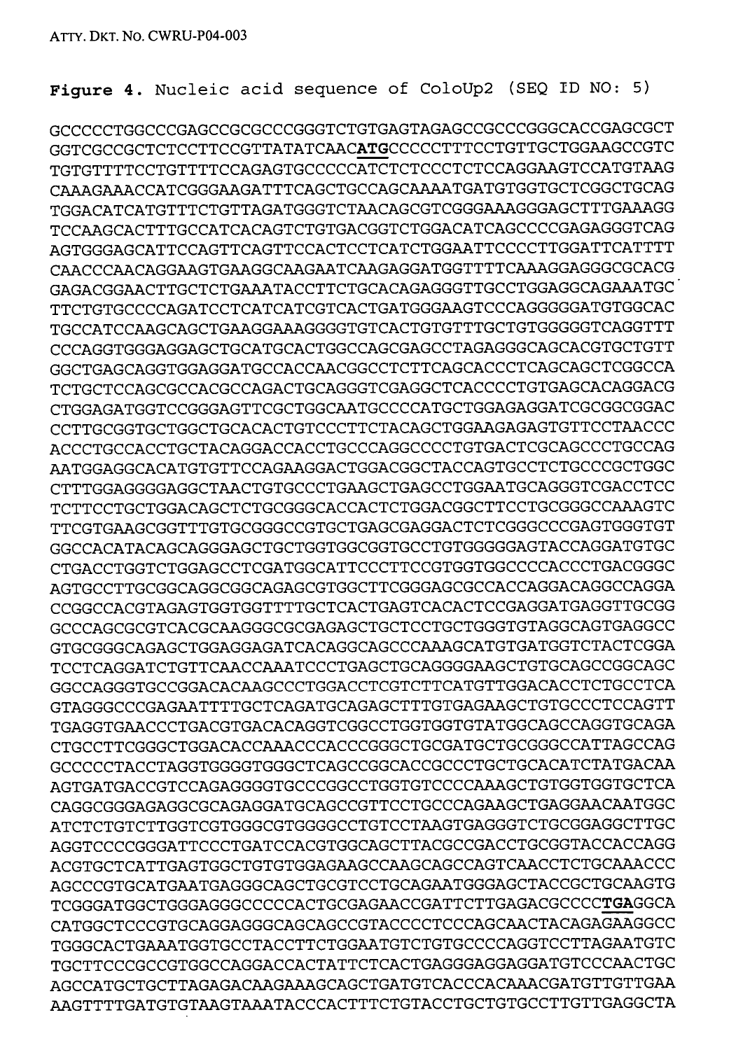

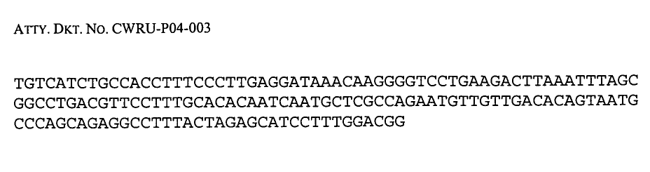

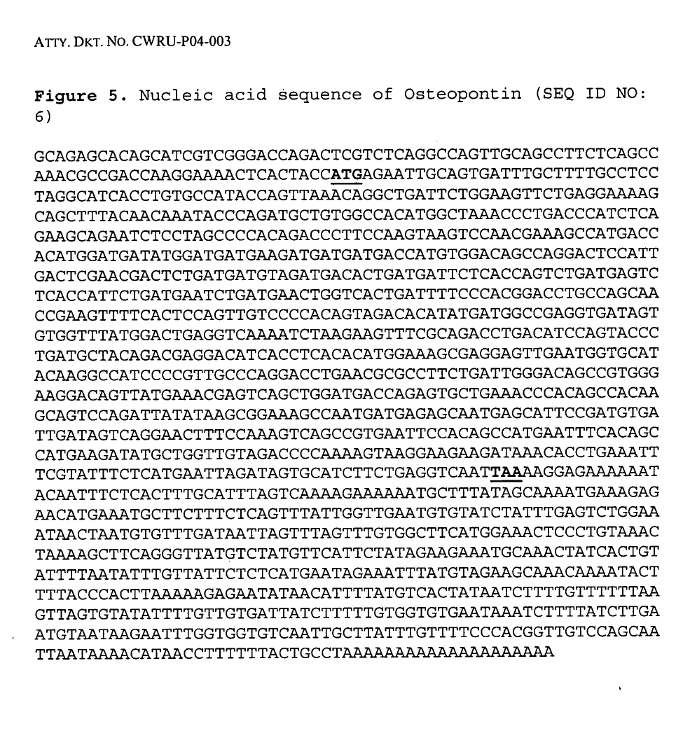

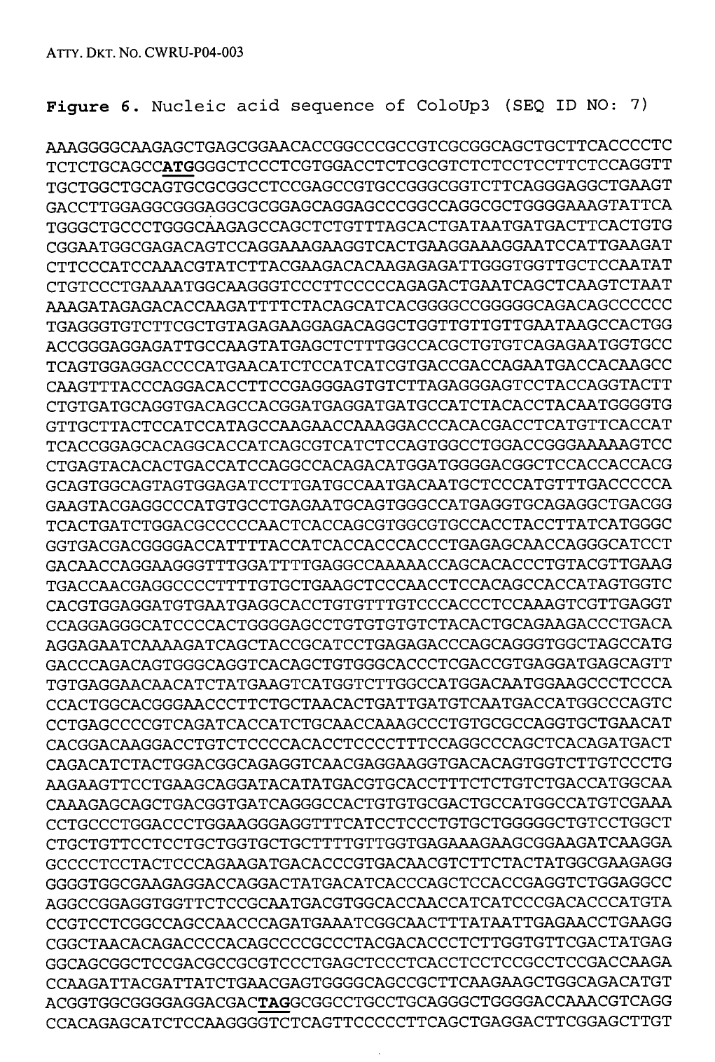

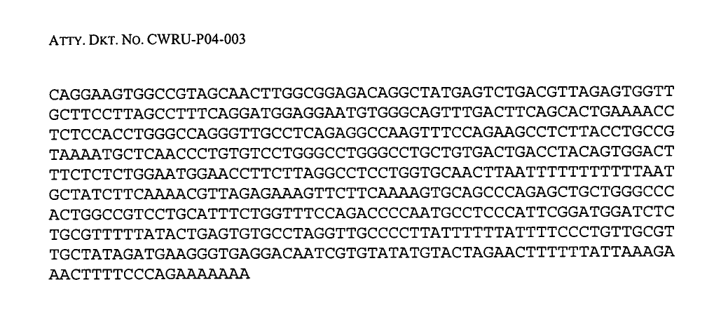

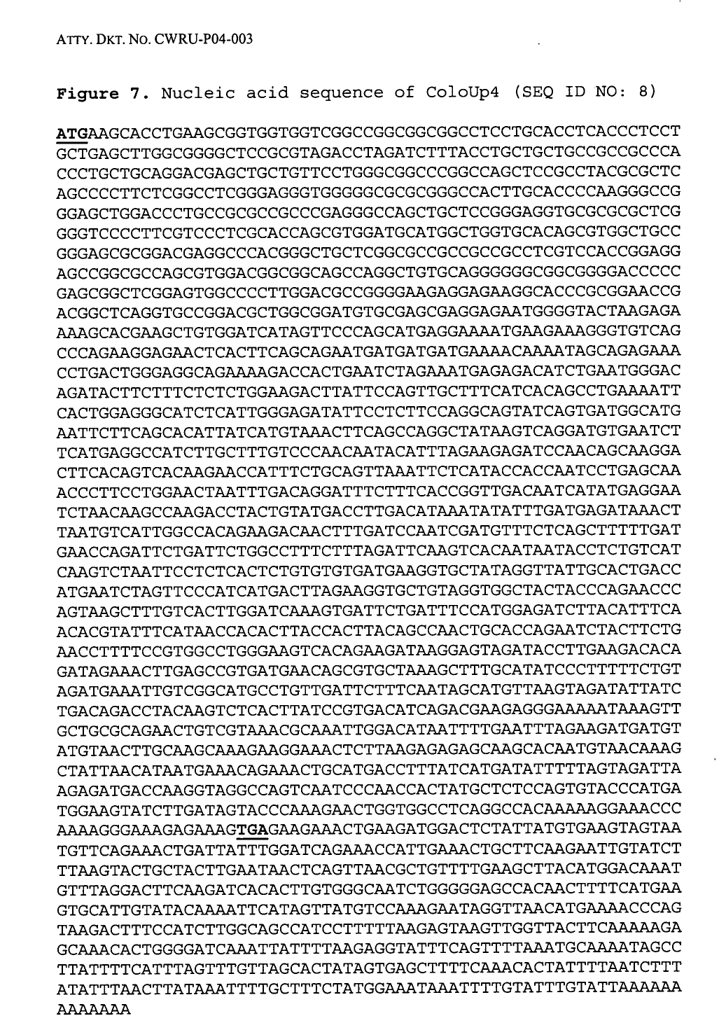

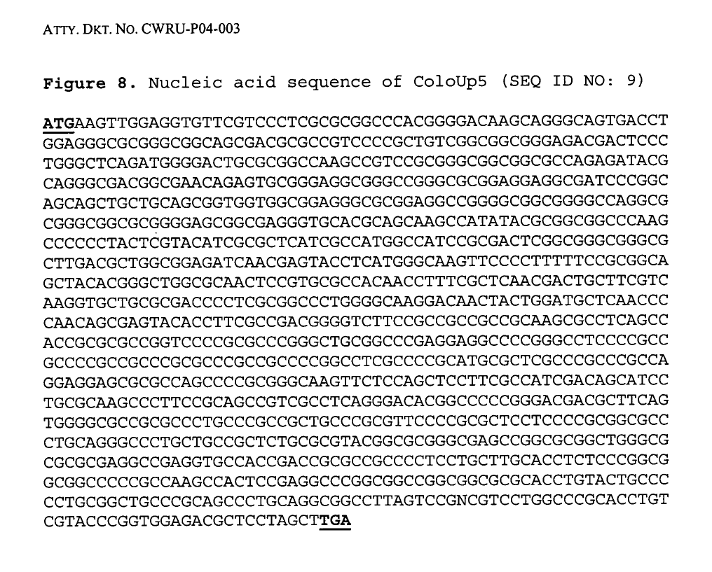

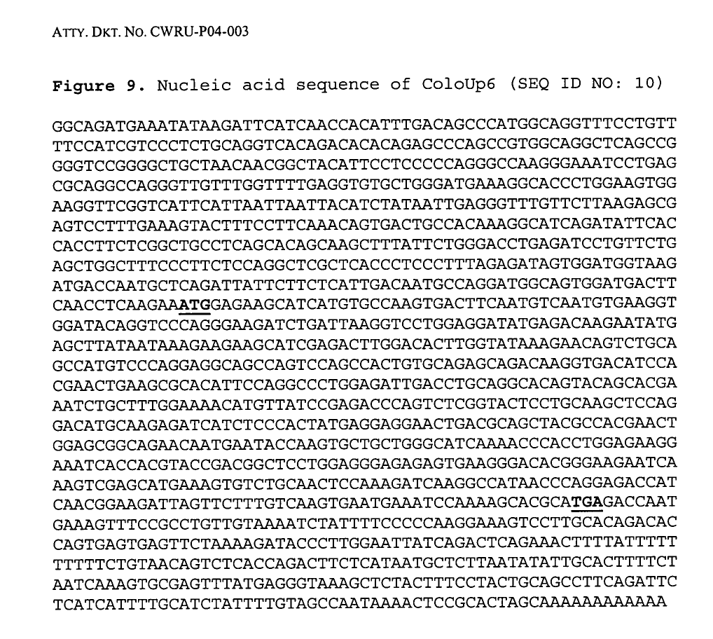

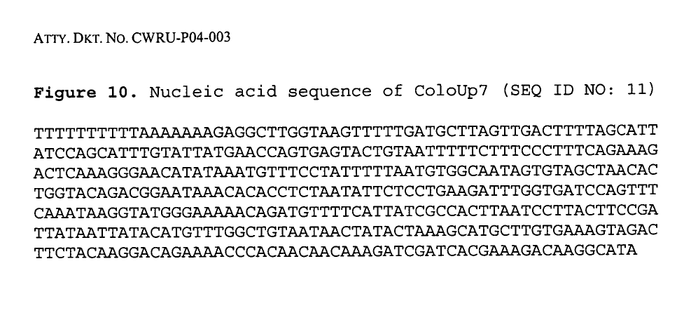

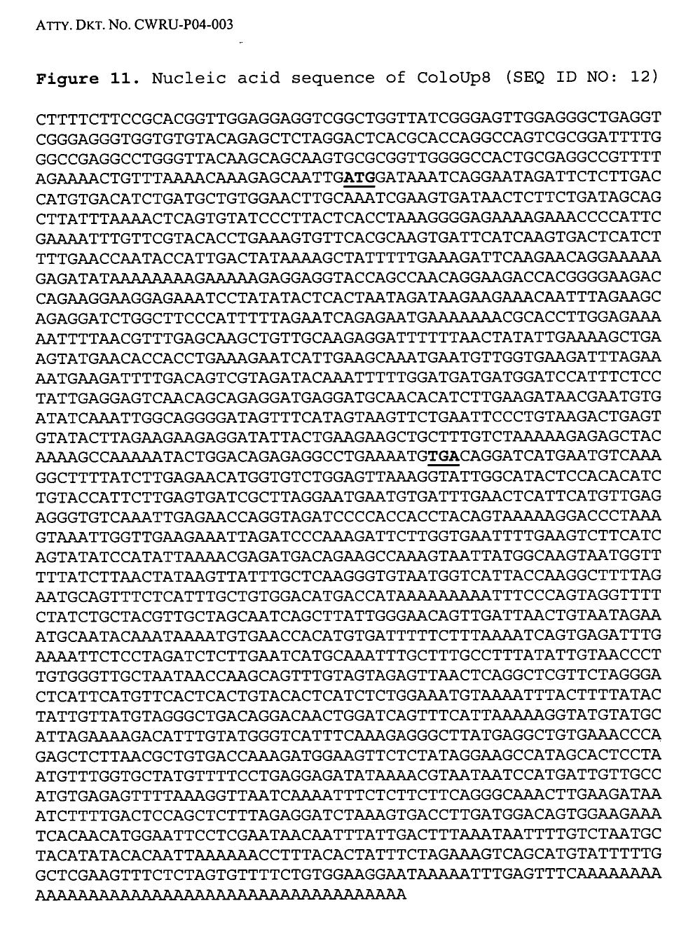

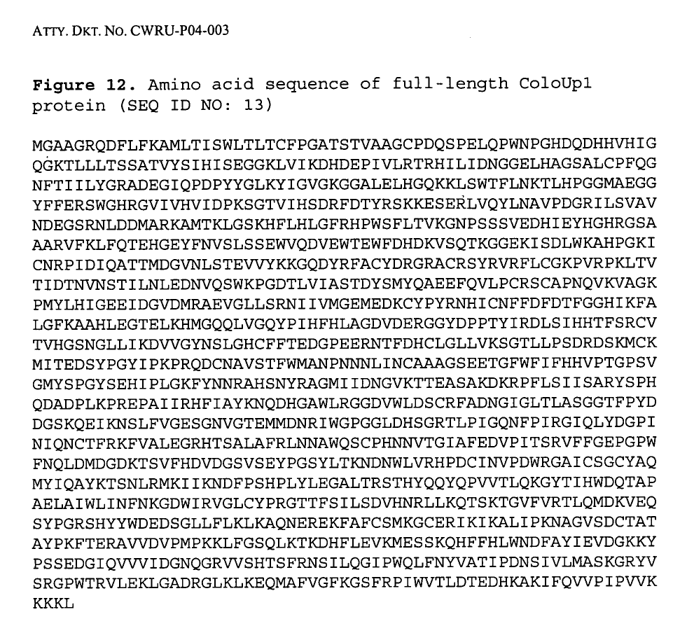

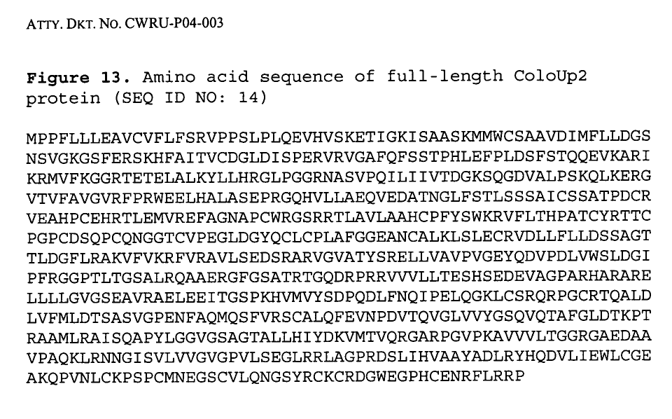

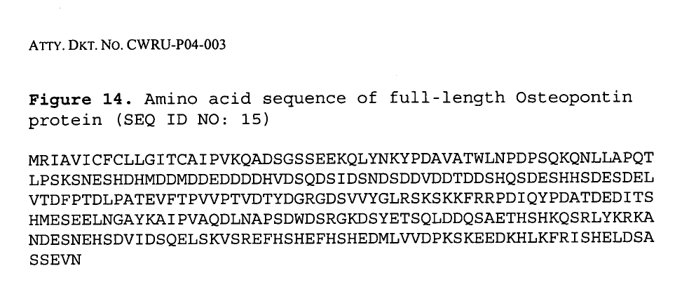

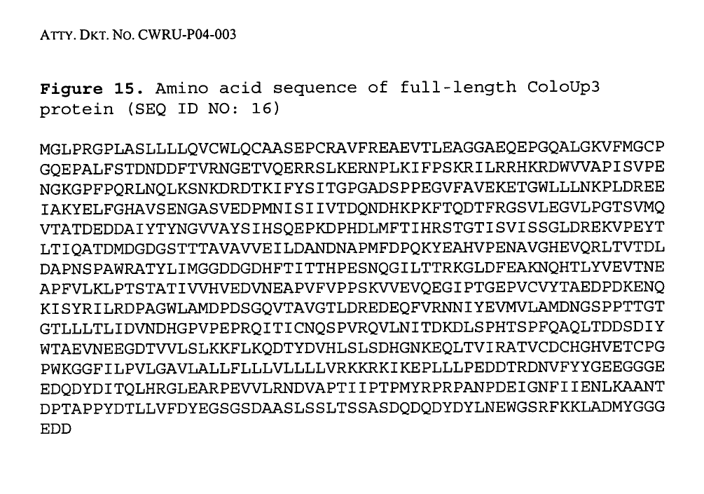

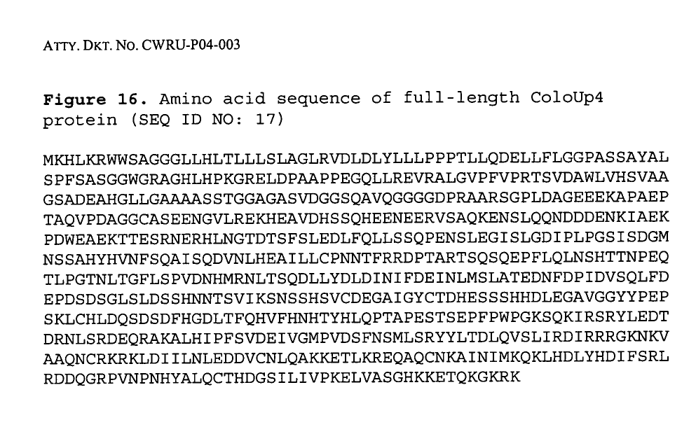

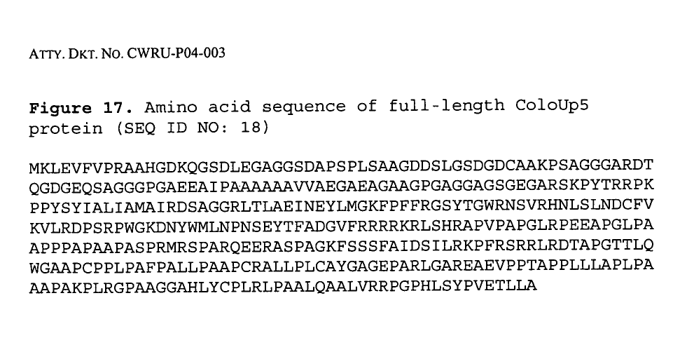

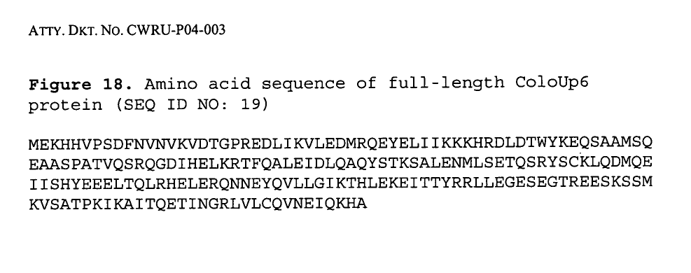

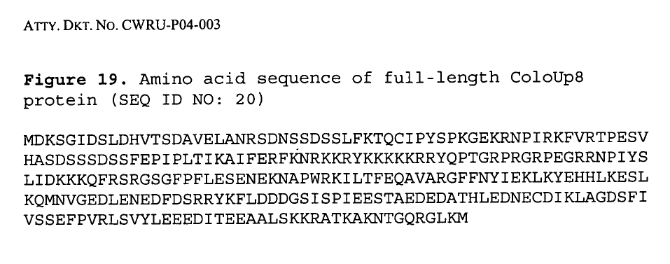

Probe ColoUp2 was designed to recognize transcripts corresponding to a noncoding EST, Genbank entry A1357412, Unigene entry Hs.157601. By 5′ RACE, database assembly, and ultimately RT-PCR, we cloned from a colon cancer cell line a novel protein encoding RNA transcript whose noncoding 3′ UTR was shown to correspond to the ColoUp2 specified EST. This full length coding sequence was determined by RT-PCR amplification from colon cancer cell line Vaco503 and sequences are provided in FIG. 4.

ColoUp2 is a “class identifier” (that is, it is higher in all colon cancer samples than in all normal colon samples), it is not-expressed in normal body tissues and it contains a signal sequence predicting that the protein product will be secreted (as well as several other recognizable protein motifs including domains from the epidermal growth factor protein and from the Von Willebrands protein).

### EXAMPLE 6

### Confirmed Gene Expression Pattern of ColoUp2

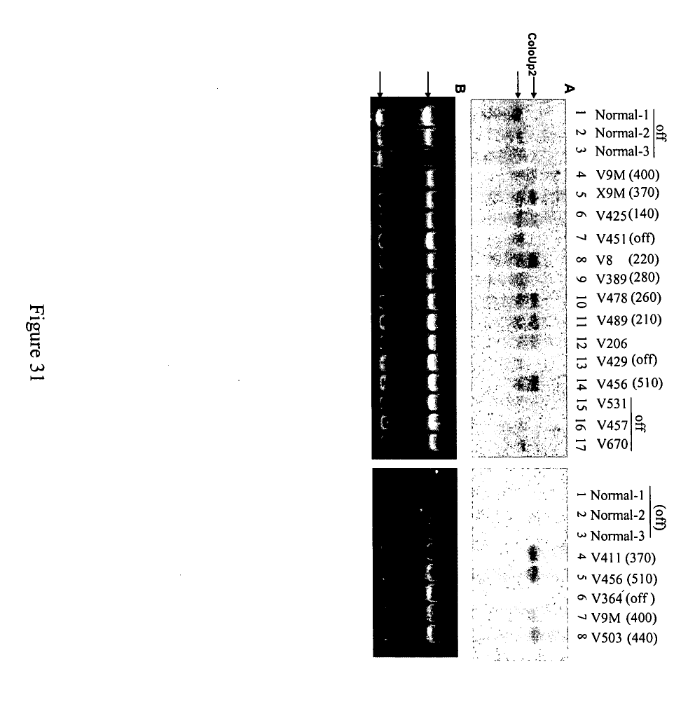

FIG. 31 shows a northern analysis using the cloned ColoUp2 cDNA that identifies a transcript running above the large ribosomal subunit (to which the probe cross hybridizes) that is not expressed in normal colon tissue samples and is expressed in the majority of group of colon cancer cell lines. Panel A of the figure shows the northern hybridization. The red arrow designates the ColoUp2 transcript. Above each lane is the name of the sample and the level (in parenthesis) of ColoUp2 expression recorded. The black arrow designates the cross hybridizing ribosomal large subunit. Panel B shows the eithidum bromide stained gel corresponding to the blot, and the black arrows designate the large and small ribosomal subunits.

### EXAMPLE 7

### ColoUp2 is a Secreted Protein

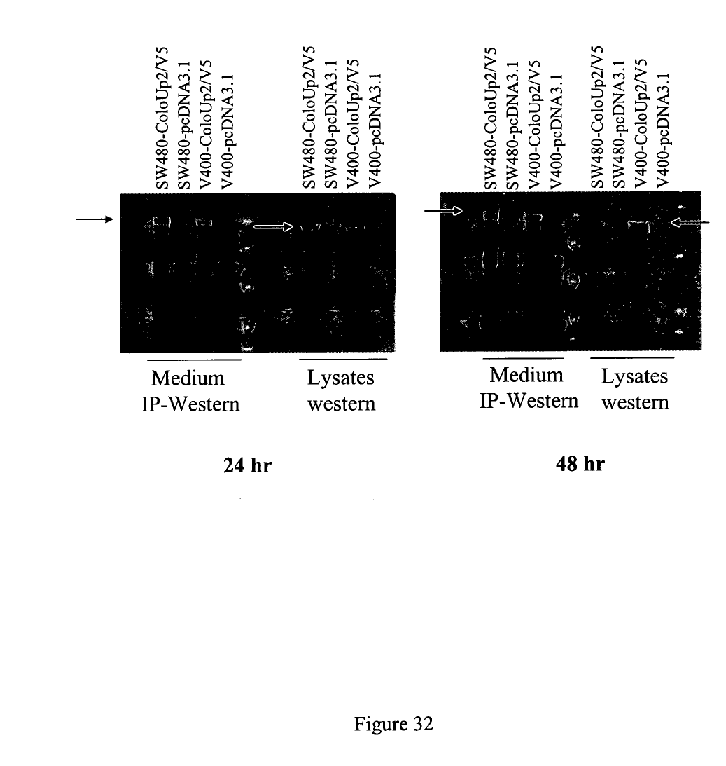

The cloned ColoUp2 colonic transcript was inserted into a cDNA expression vector with a C-terminal V5 epitope tag. FIG. 32 shows a summary of the behavior of the tagged protein expressed by transfection of the vector into SW480 and Vaco400 cells. An anti V5 western blot shows (red arrows) expression of the transfected tagged protein detected in the lysate of a pellet of transfected cells (lysates western panel, lanes labeled ColoUp2/V5) which is absent in cells transfected with a control empty expression vector (lanes labeled pcDNA3.1). Moreover, serial immunoprecipitation and western blotting of V5 tagged protein from media in which V400 and SW480 cells were growing (which had been clarified by centrifugation prior to immunoprecipatation) also clearly demonstrates secretion of the ColoUp2 protein into the growth medium (panel labeled medium IP-western). Antibody bands from the immunoprecipitation are also present on the IP-western blot. Detection of secreted ColoUp2 protein was shown in cells assayed both 24 hours and 48 hours after transfection.

### EXAMPLE 8

### Expression Pattern of ColoUp3-ColoUp8 and Osteopontin in Various Cell Types

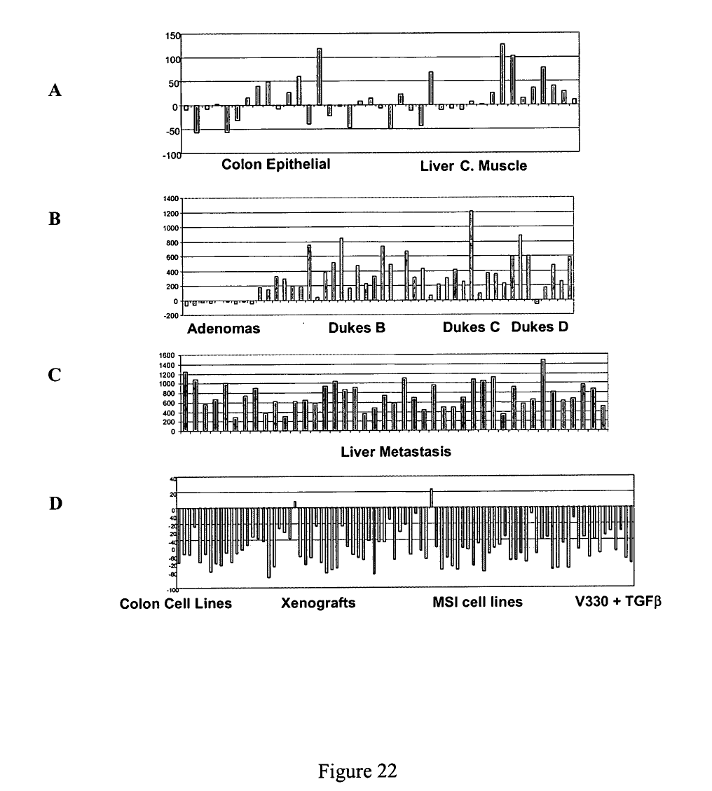

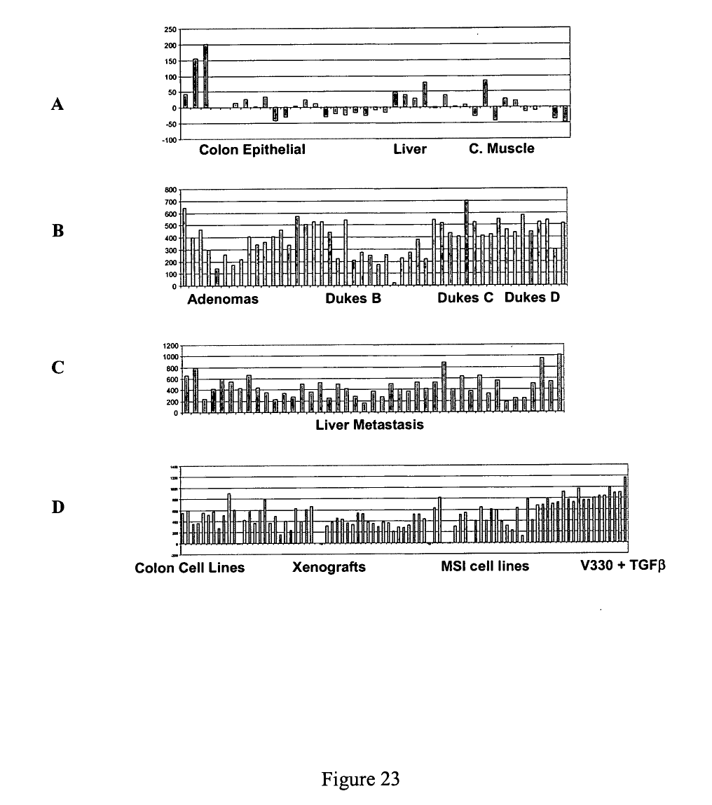

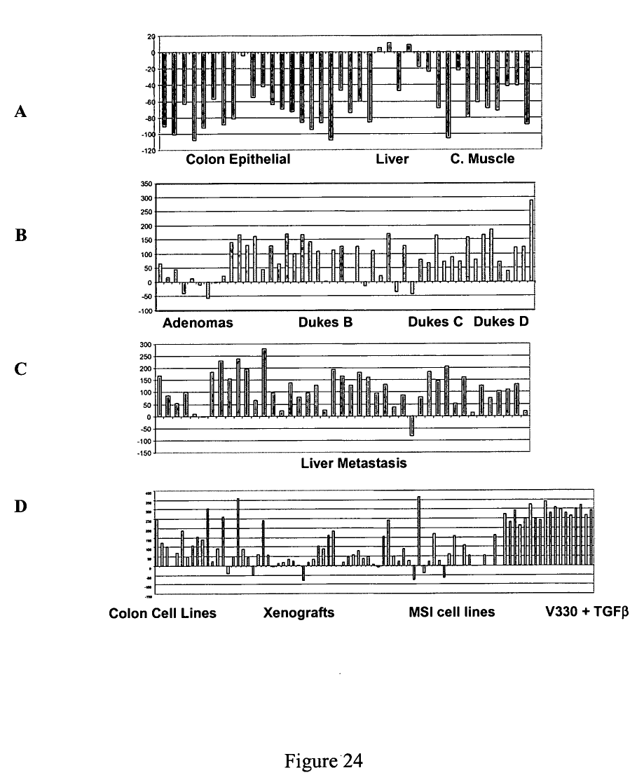

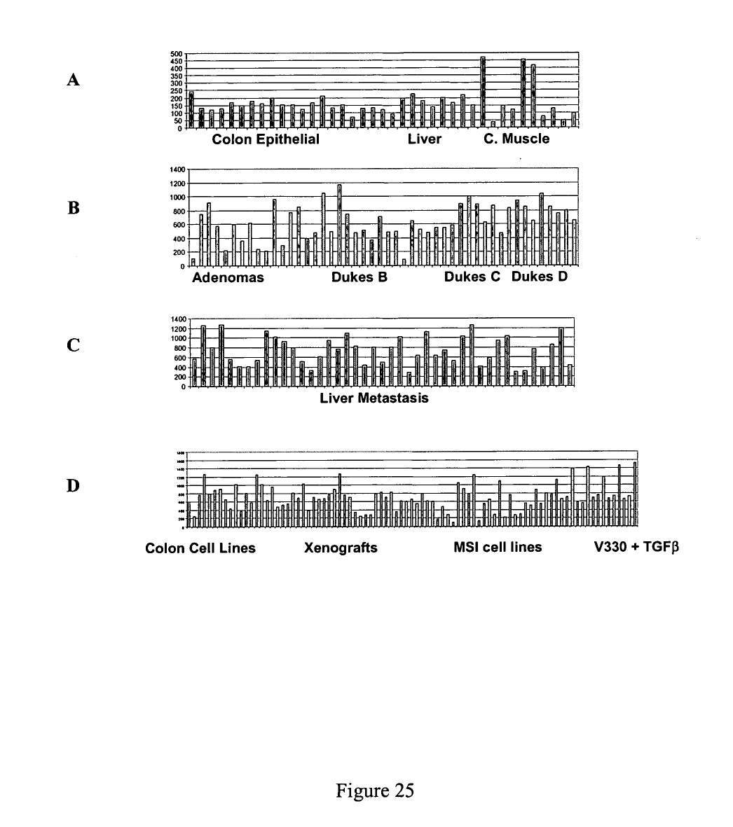

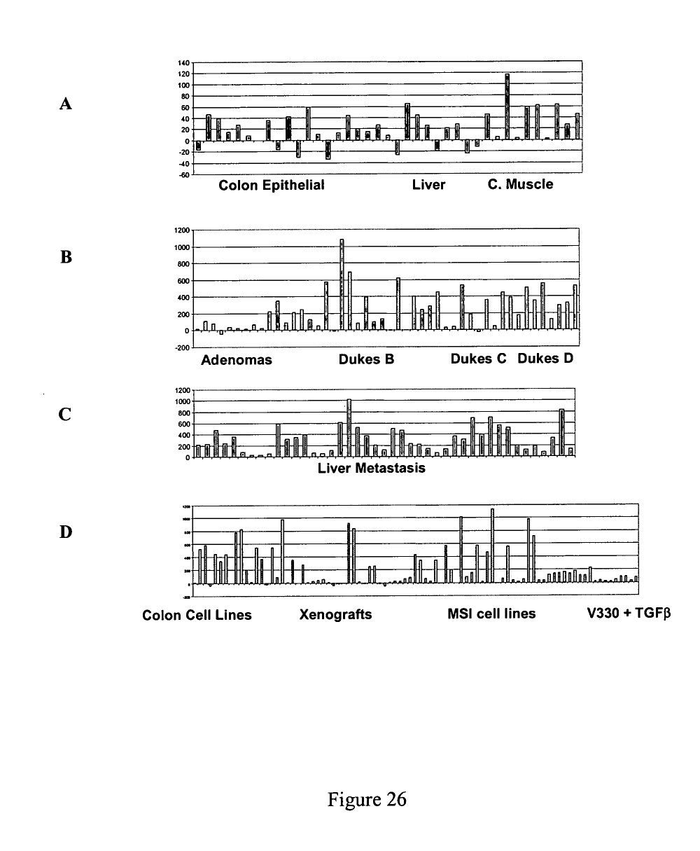

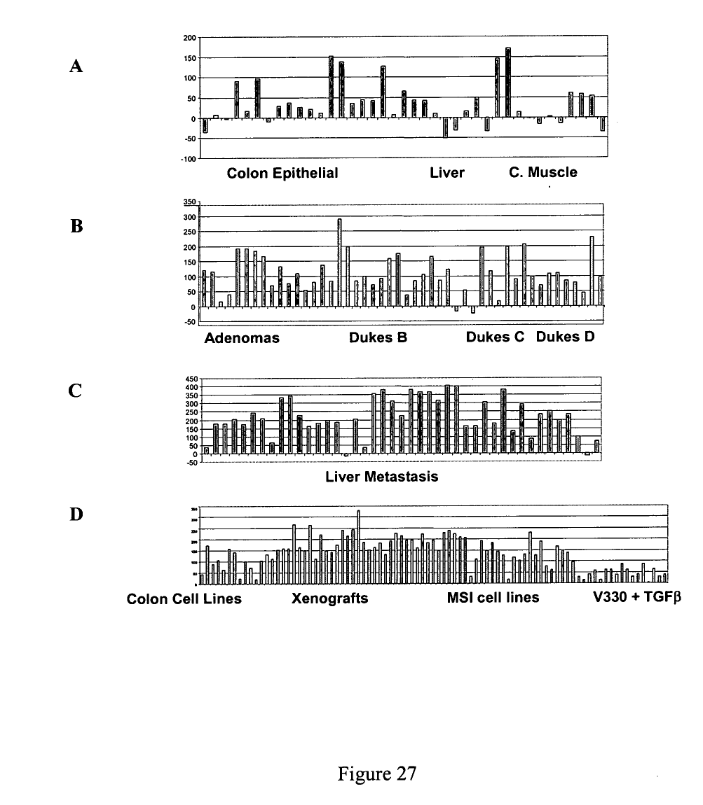

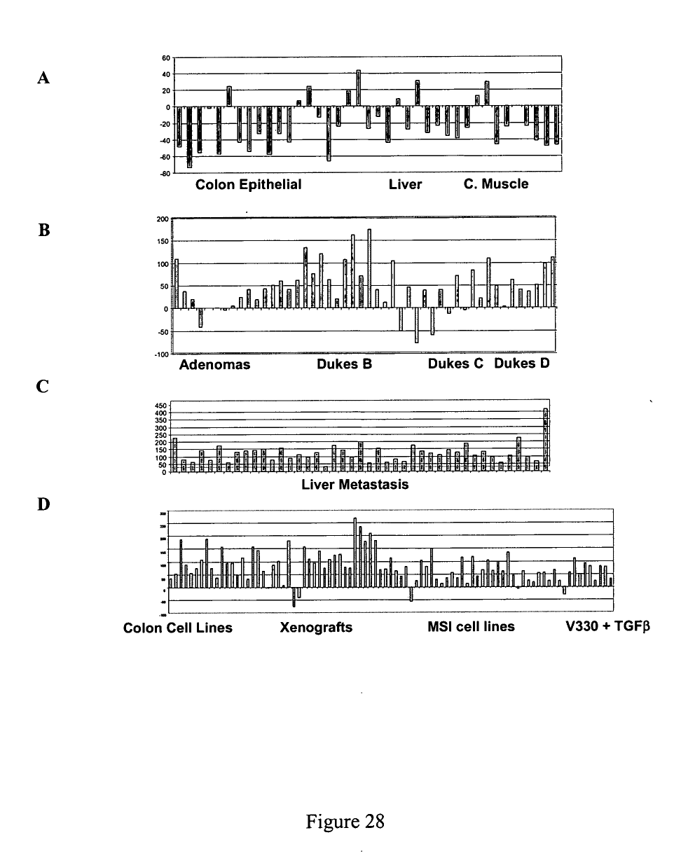

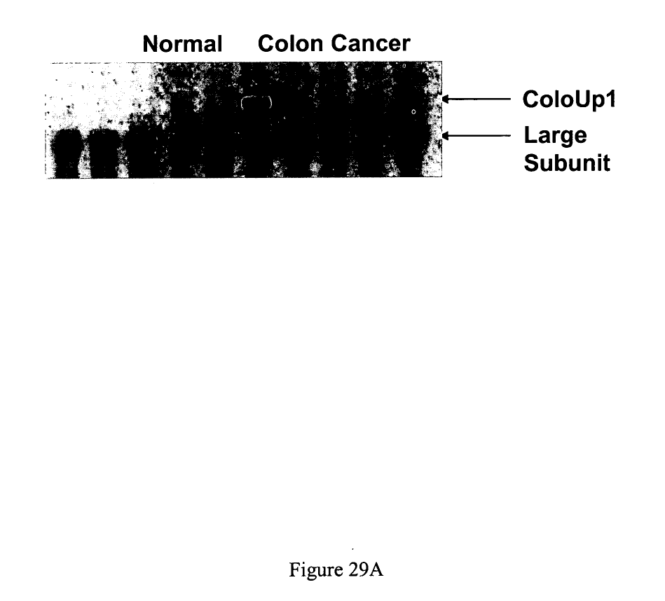

Shown in FIGS. 22-28 are the graphical displays of ColoUp3-ColoUp8 and osteopontin expression levels measured for different samples analyzed.

### EXAMPLE 9

### Confirmed Gene Expression Pattern of ColoUp5

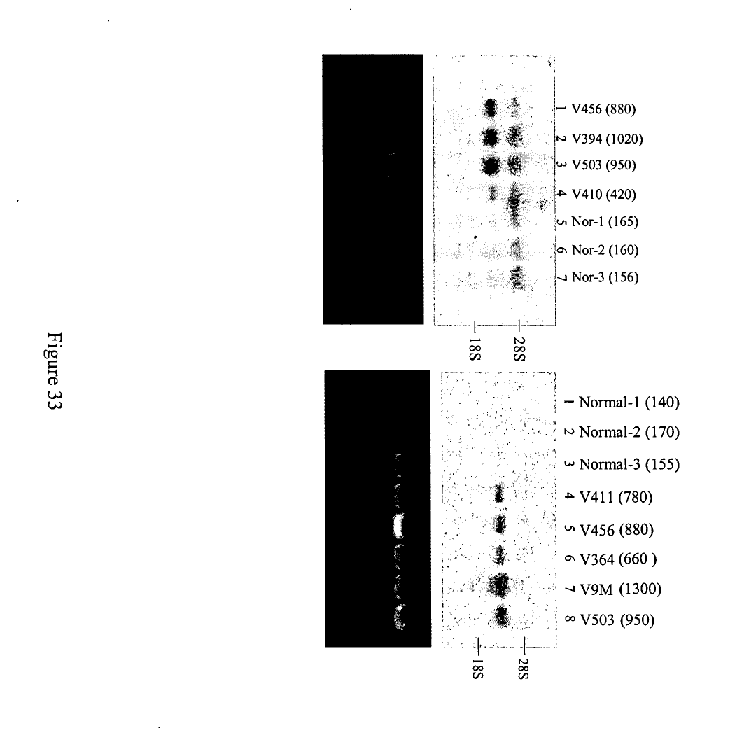

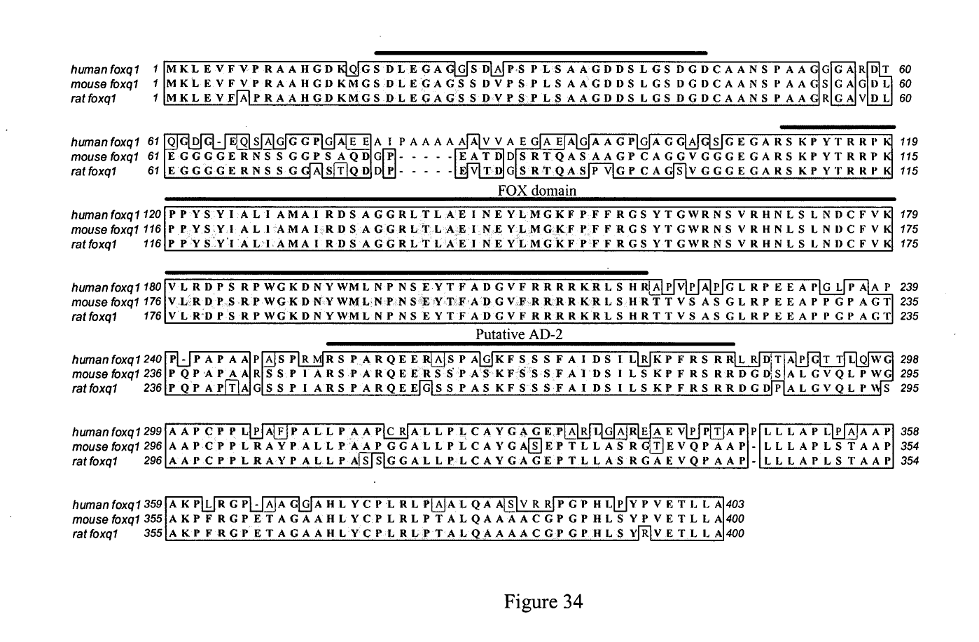

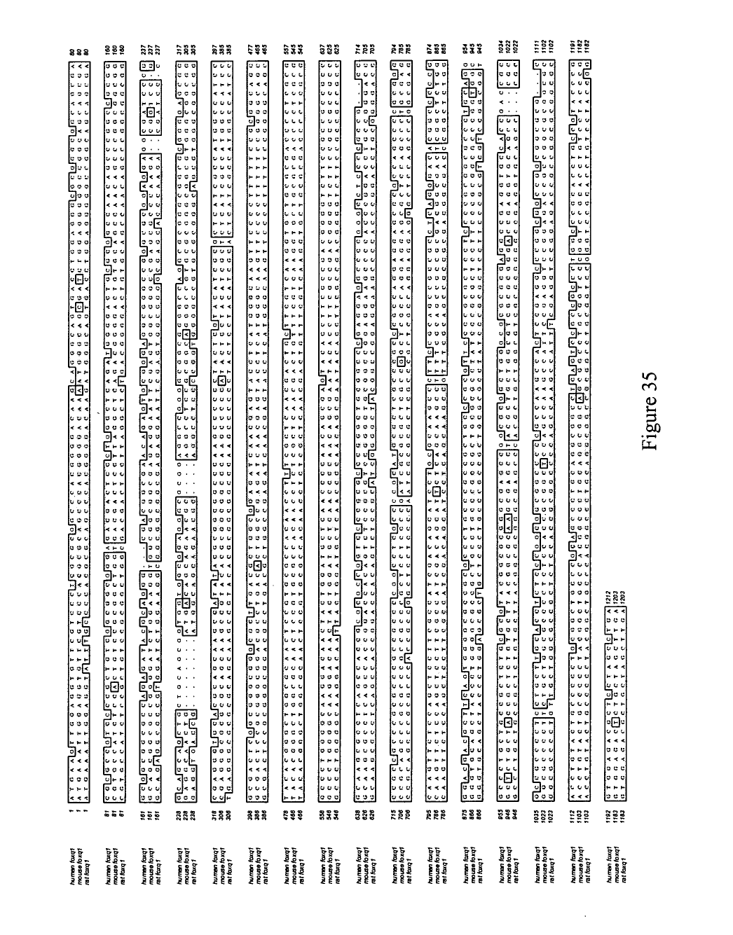

Shown in FIG. 33 is a northern blot showing that ColoUp5 is expressed in colon cancer cell lines and not expressed in non-neoplastic material. FIG. 33 shows two northern blot analysis of ColoUp5 mRNA levels in normal colon tissues and a group of colon cancer cell lines (top panels). The bottom panels show the ethidium bromide stained gel corresponding to the blot. Homologs for ColoUp5 are found in other mammals, including mouse and rat, and sequence alignments are shown in FIGS. 34 and 35.

### EXAMPLE 10

### Detection of Xenograft Derived ColoUp1 and ColoUp2 Proteins Circulating in the Blood of Mice

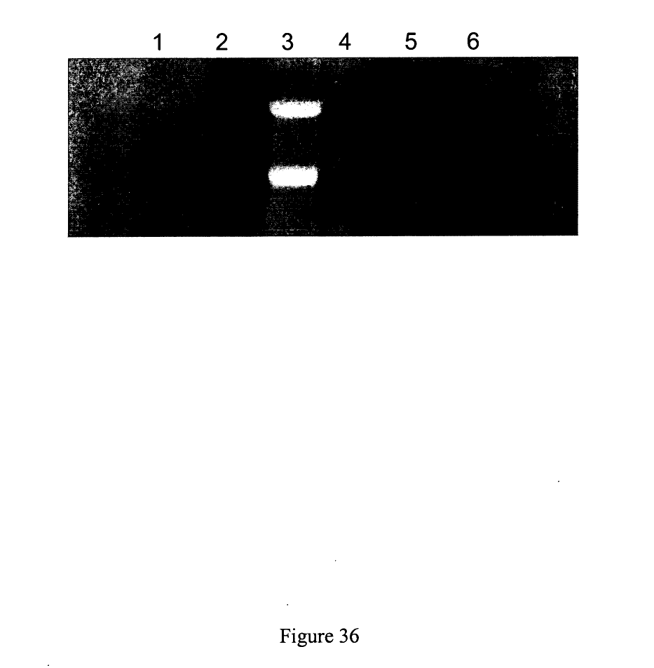

To determine that ColoUp1 and ColoUp2 proteins are effective serologic markers of colon neoplasia, we derived transfected cell lines that stably expressed and secreted V5-epitope tagged ColoUp1 and ColoUp2 proteins. These cells lines were then injected into athymic mice and grown as tumor xenografts. Mice were sacrificed and serum was obtained. V5 tagged proteins were then precipitated from the serum using beads conjugated to anti-V5 antibodies. Precipitated serum proteins were run out on SDS-PAGE, and visualized by western blotting using HRP-conjugated anti-V5 antibodies (thereby eliminating visualization of any contaminating mouse immunoglobulin). FIG. 36 shows detection of circulating ColoUp2 protein in mouse serum. The ColoUp2 protein is secreted as 2 bands of 85 KD and 55 KD in size, of which the 55 KD band predominates in the serum. The 55 KD band is presumably a processed form of the 85 KD band. This observation demonstrates that, in this mouse model, ColoUp2 is indeed a secreted marker of colon cancers and adenomas, and that ColoUp2 can gain access to and circulate stably in patient serum. This observation provides the surprising result that a processed fragment of ColoUp2 is the predominant serum form of the protein and therefore detection reagents targeted to this portion would be particularly suitable for diagnostic testing.

A time course experiment showed that ColoUp2 protein was detectable in mouse blood at the earliest time assayed, 1 week after injection of ColoUp2 secreting colon cancer cells, at which time xenograft tumor volume as only 100 mm³.

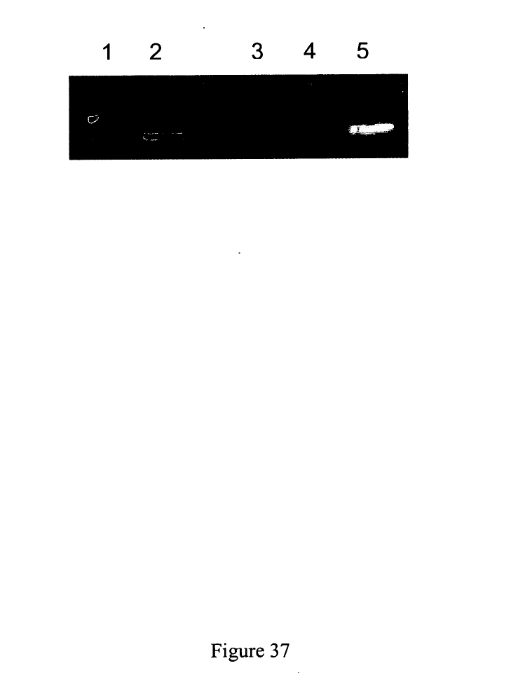

Similar observations were also made for ColoUp1, as shown in FIG. 37.

### EXAMPLE 11

### Purification of ColoUp1 and ColoUp2 Proteins

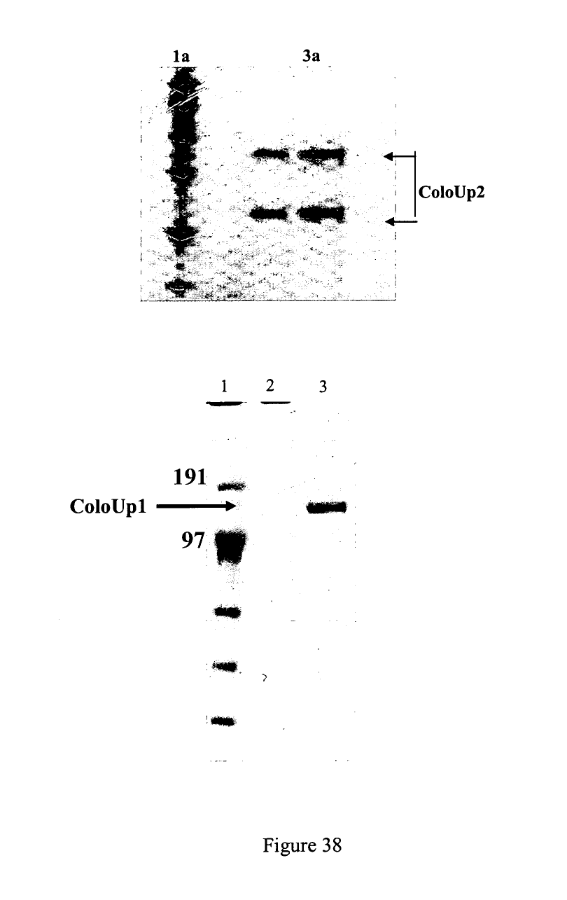

In order to develop monoclonal antibodies against native ColoUp1 and ColoUp2 proteins, we devised a protocol for purification on Ni-NTA agarose (QIAGEN) nickel beads of recombinant His tagged ColoUp1 and ColoUp2 proteins from the media supemate of SW480 cells engineered to express these proteins. Currently we have purified both ColoUp1 and ColoUp2 proteins to sufficient purity to generate antibodies. As shown in FIG. 38, a Coomassie blue stained gel of purified ColoUp2 shows only the 85 KD and 55 KD size bands that correspond to the tagged ColoUp2 proteins visualized on western blot. Similarly, a Coomassie blue stained gel of purified ColoUp1 shows the preparation is highly purified and composed of a single 180 KD band that corresponds perfectly to the size band seen on western blotting of the epitope tagged ColoUp1 protein. Thus we have purified ColoUp2 and ColoUp1 to sufficient homogeneity and yield. Scaled up purification of these proteins from a 50 liter media preparation should yield 2.5 mg of protein, more than adequate for immunizing mice and screening fusion supernates for development of monoclonal antibodies specific for native ColoUp1 and ColoUp2.

### EXAMPLE 12

### Measuring Apical and Basolateral Secretion of ColoUp1 and ColoUp2

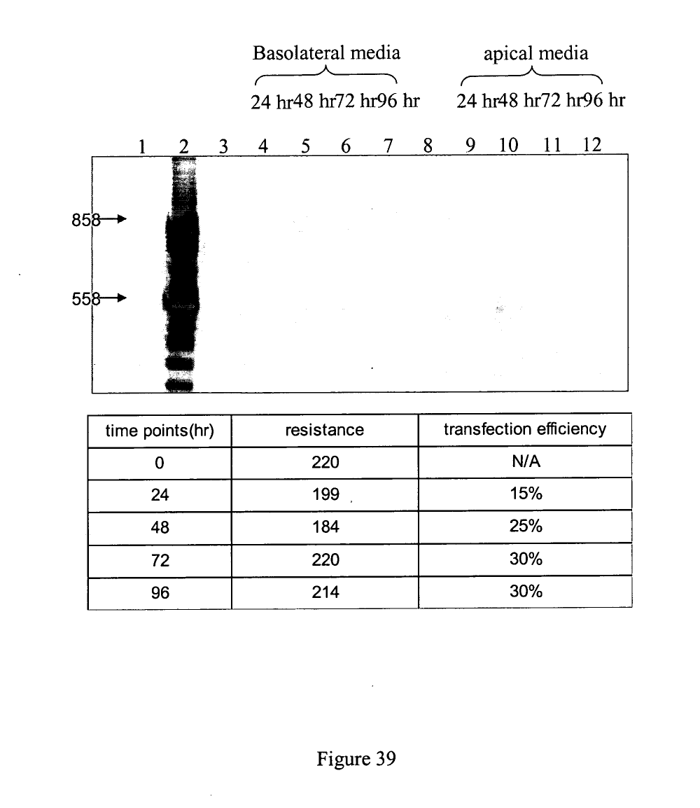

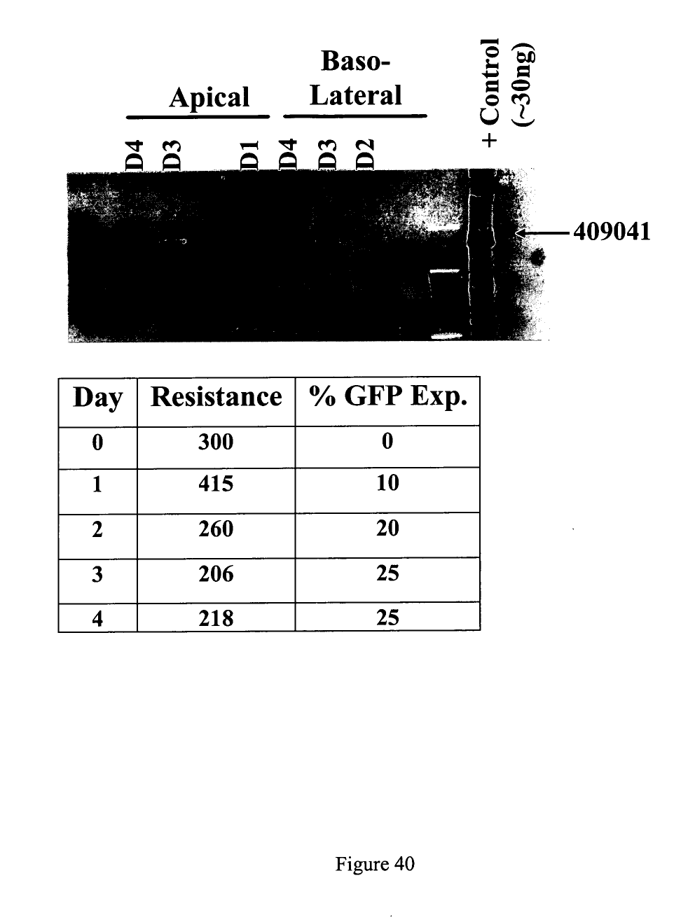

We expected that ColoUp2 will serve as a serologic marker detection not only of colon cancers but also of large colon adenomas that also express ColoUp2. Adenomas, unlike colon cancers, are non-invasive. Thus, for adenomas to move ColoUp2 proteins into the circulation they would need to secrete this protein from the basolateral cell surface facing capillaries and lymphatics, rather than from the apical cell surface facing the colon lumen. To determine the polarity of ColoUp2 secretion we transiently transfected a monolayer of polarized Caco2 colon cancer cells with an expression vector for V5-epitope tagged ColoUp2 protein. This cell monolayer was grown in transwell dishes on filters that separate an upper transwell chamber (representing media exposed to the apical surface of the monlayer) from a lower transwell chamber (representing media exposed to the basolateral surface of the monolayer). Integrity of the sealing of the monolayer was assayed by measuring electrical resistance across the filters, and efficiency of transient transfection was monitored by expression of a gfp marker. Media from upper and lower chambers was harvested at 24, 48, 72, and 96 hours post transfection, and secreted tagged ColoUp2 protein was detected by western analysis directed against the V5 epitope tag. As FIG. 39 shows, characteristic 85 KD and 55 KD secreted forms of ColoUp2 were detected in media sampling the basolateral monolayer compartment at all time points assayed. At a single time point, 48 hours, ColoUp2 was additionally detected in media representing the apical secretion face; however, a dip in the transfilter electrical resistance at 48 hours suggests the likelihood of some leaking across the monolayer at this time point. Certainly, the data clearly shows secretion of ColoUp2 into the basolateral monolayer compartment, and hence establishes ColoUp2 as demonstrating the requisite biology for a candidate serologic marker of colon adenomas.

As was done for ColoUp2, ColoUp1 expression vectors were used to transiently transfect Caco2 cell monolayers grown on transwell filters. Secretion of ColoUp1 was then assayed in media collected respectively from the upper and lower transwell chambers. Western blot assays demonstrated equal secretion of ColoU1 from both apical and basolateral monolayer surfaces. Studies of ColoUp1 were done in parallel with those of ColoUp2, and electrical resistance of the ColoUp1 monolayers exceeded that of the ColoUp2 monolayers, supporting that the ColoUp1 transfected monolayers were well sealed. Additionally, levels of secreted ColoUp1 protein were similar to those of secreted ColoUp2, suggesting that ColoUp1 secretion by both apical and basolateral compartments was not simply due to overexpression.. Accordingly, we predict that native ColoUp1 protein is likely secreted at least in part from the basolateral epithelial face, and hence should be detectable as a serologic marker of large colon adenomas.

### EXAMPLE 13

### Determining the Sequence of the 55 kDa ColoUp2 Fragment

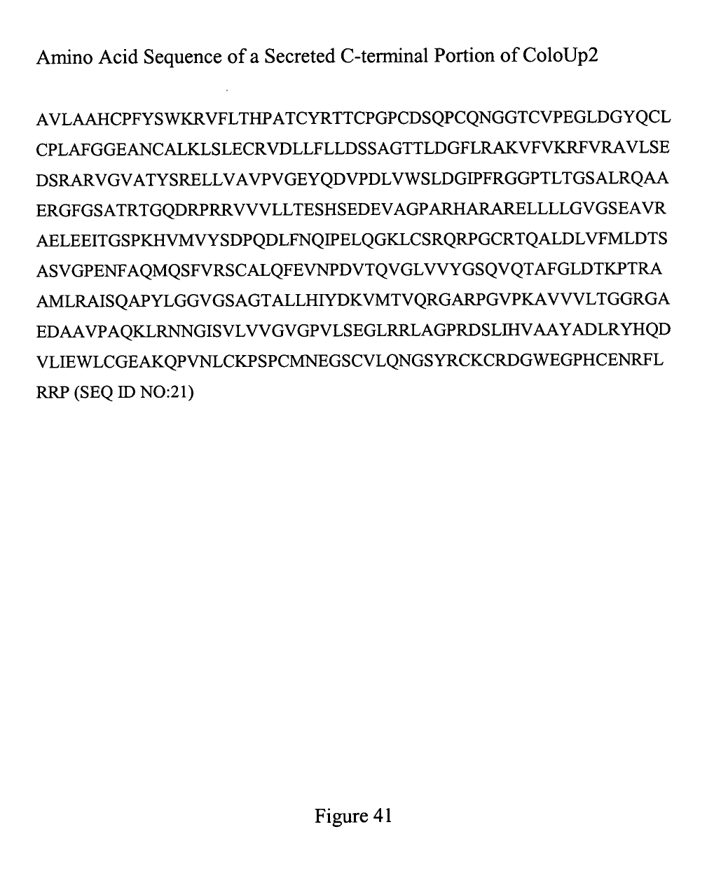

The protein sequence of C-terminal fragment of ColoUp2 that is secreted by human cell lines and detected as predominant fragment in blood (488 aa) was determined. As described above, we have found on western blots and on purified preparations of C-terminal epitope tagged (V5-His epitope) ColoUp2 protein secreted by transfected human colon cancer cells, both a full sized band of approximately 90 kDa and a smaller approximately 55 kDa C-terminal fragment (as demonstrated by the retention of the C-terminal epitope tag). Moreover, when these cells were injected into athymic mice, the 55 kDa C-terminal tagged protein was the predominant species detected as circulating in the mouse blood, when mouse serum is analyzed by serial immunoprecipitation and western blot analysis directed against the V5 tag. The precise location of the cleavage site accounting for the C-terminal fragment was established by excising the acrylamide gel band containing the purified C-terminal fragment and performing mass spectroscopy analysis of tryptic fragments from the protein. A peptide of sequence AVLAAHCPFYSWK was present only in the digest of the 55 KD fragment, but was absent from the digest of the full length protein, demonstrating that this peptide corresponded to the unique amino terminus of the 55 KD fragment. The complete sequence of the 55 KD C-terminal fragment is shown in FIG. 41.

Incorporation by Reference

All publications and patents mentioned herein are hereby incorporated by reference in their entirety as if each individual publication or patent was specifically and individually indicated to be incorporated by reference. In case of conflict, the present application, including any definitions herein, will control.

Equivalents

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A method for detecting whether a subject to likely to have a colon neoplasia comprising: detecting in a biological sample from said subject one or more polypeptides selected from among: one or more ColoUp1 polypeptides, wherein the presence of said one or more polypeptides is indicative of colon neoplasia.

2. The method of claim 1, wherein the ColoUp1 polypeptide is encoded by a nucleic acid sequence that is at least 95% identical to the nucleic acid sequence of SEQ ID No: 4.

3. The method of claim 1, wherein the ColoUp1 polypeptide is encoded by a nucleic acid sequence that is at least 98-99% identical to the nucleic acid sequence of SEQ ID No: 4.

4. The method of claim 1, wherein the ColoUp1 polypeptide is encoded by SEQ ID No: 4.

5. The method of claim 1, wherein the ColoUp1 polypeptide is encoded by a nucleic acid sequence that hybridizes under stringent conditions to a nucleic acid sequence of SEQ ID No: 4.

6. The method of claim 2, wherein the ColoUp1 polypeptide comprises a polypeptide sequence that is at least 95% identical to the amino acid sequence as set forth in any one of SEQ ID No: 1, SEQ ID No: 2 and SEQ ID No: 13.

7. The method of claim 1, wherein said biological sample is selected from the group consisting of whole blood or a fraction thereof.

8. The method of claim 1, wherein said biological sample is selected from the group consisting of urine or stool samples.

9. The method of claim 1, wherein said biological sample is a blood sample.

10. The method of claim 1, wherein the polypeptide is detected by binding to an antibody.

11. A recombinant, isolated nucleic acid comprising a nucleic acid sequence that is at least 95% identical to a nucleic acid sequence of SEQ ID No: 4, or a complement thereof.

12. The recombinant, isolated nucleic acid of claim 11, wherein the recombinant nucleic acid comprises a nucleic acid sequence that is identical to the nucleic acid of SEQ ID No: 4 or a complement thereof.

13. An expression construct comprising the recombinant, isolated nucleic acid of claim 11.

14. A cell comprising the expression construct of claim 13.

15. A method of preparing a ColoUp1 polypeptide comprising,
(a) obtaining a cell of claim 14;
(b) culturing the cell under conditions that promote production of the polypeptide encoded by the recombinant nucleic acid;
(c) obtaining a cellular fraction that comprises the polypeptide encoded by the recombinant nucleic acid.

16. A method of claim 15, further comprising purifying the cellular fraction of (c) to obtain a substantially pure ColoUp2 polypeptide.

17. A recombinant polypeptide comprising an amino acid sequence that is at least 95% identical to a sequence selected from the group consisting of SEQ ID Nos: 1 and 2.

18. The recombinant polypeptide of claim 17, wherein the recombinant polypeptide comprises an amino acid sequence that is identical to a sequence selected from the group consisting of SEQ ID Nos: 1 and 2.

19. The recombinant polypeptide of claim 17, wherein the recombinant polypeptide consists of an amino acid sequence that is identical to a sequence selected from the group consisting of SEQ ID Nos: 1 and 2.

20. An antibody that binds specifically to a ColoUp1 polypeptide of claim 17.

21. The antibody of claim 20, wherein the antibody is a monoclonal antibody.

22. The antibody of claim 20, wherein the antibody is effective for binding specifically to the ColoUp2 polypeptide in a blood sample.
